(19) United States
(12) Patent Application Publication
(10) Pub. No.: US 2025/0115923 A1
(43) Pub. Date: Apr. 10, 2025

(54) SIMULTANEOUS GENE EDITING AND HAPLOID INDUCTION

(71) Applicant: SYNGENTA CROP PROTECTION AG, BASEL (CH)

(72) Inventors: Qiudeng QUE, Research Triangle Park, NC (US); Timothy KELLIHER, Research Triangle Park, NC (US)

(21) Appl. No.: 18/980,635

(22) Filed: Dec. 13, 2024

Related U.S. Application Data
(63) Continuation of application No. 18/060,362, filed on Nov. 30, 2022, now Pat. No. 12,195,737.
Application No. 18/060,362 is a continuation of application No. 16/678,286, filed on Nov. 8, 2019, now abandoned.
Application No. 16/678,286 is a continuation of application No. 16/245,923, filed on Jan. 11, 2019, now Pat. No. 10,519,456.
Application No. 16/245,923 is a continuation of application No. 15/901,464, filed on Feb. 21, 2018, now Pat. No. 10,285,348.
Application No. 15/901,464 is a continuation of application No. PCT/US2017/064512, filed on Dec. 4, 2017.
(60) Provisional application No. 62/429,260, filed on Dec. 2, 2016.

(51) Int. Cl. C12N 15/82; A01H 1/02; C12N 9/22; C12N 9/24; C12N 9/80; C12N 15/11

(52) U.S. Cl. CPC C12N 15/8213; C12N 15/102; C12N 15/113; C12N 15/65; C12N 15/8218; C12N 2310/20

(57) ABSTRACT

The presently disclosed subject matter relates to using a haploid inducing line (whether existing or created) and transforming the haploid line so that it encodes cellular machinery capable of editing genes. The transformed haploid inducing line is used as a parent in a cross between two plants. During pollination, the parental gametes fuse to form an embryo; and the gene editing machinery is also delivered to the embryo at this time. During embryonic development, one set of parental chromosomes are lost, and the gene editing machinery operates on the remaining set of chromosomes. Thus, at least one haploid progeny with edited genes is produced from the cross.

This application claims the benefit under 35 U.S.C. § 120 of pending U.S. patent application Ser. No. 18/060,362, filed Nov. 30, 2022, which claims the benefit under 35 U.S.C. § 120 of abandoned U.S. patent application Ser. No. 16/678,286, filed Nov. 8, 2019, which claims benefit under 35 U.S.C. § 120 of U.S. patent application Ser. No. 16/245,923, filed Jan. 11, 2019, now U.S. Pat. No. 10,519,456, which claims the benefit under 35 U.S.C. § 120 of U.S. application Ser. No. 15/901,464, filed Feb. 21, 2018, now U.S. Pat. No. 10,285,348, which claims the benefit under 35 U.S.C. § 120 of International Application No. PCT/US2017/064512, filed Dec. 4, 2017 and designating the U.S., which claims priority under 35 U.S.C. § 119 to U.S. Provisional Application 62/429,260, filed Dec. 2, 2016, all of which are incorporated herein by reference in their entirety.

### FIELD OF THE INVENTION

This invention is related to the field of plant biotechnology, specifically agriculture biotechnology and gene editing, as well as plant breeding. The presently disclosed subject matter relates to using a haploid inducing line (whether existing or created) and transforming the haploid inducing line so that it contains DNA coding for cellular machinery capable of editing genes.

### SEQUENCE LISTING

This application is accompanied by a sequence listing entitled 086879-1359385_ST26.xml, created Nov. 30, 2022, which is approximately 492,637 bytes in size. This sequence listing is incorporated herein by reference in its entirety. This sequence listing is submitted herewith via EFS-Web, and is in compliance with 37 C.F.R. § 1.824(a)(2)-(6) and (b).

### BACKGROUND

Targeted mutagenesis (also known as “gene editing”) is a very important technology to crop breeding. There are numerous methods to edit specific gene targets now, including CRISPR, TALEN, meganucleases, and zinc fingers. One method to introduce editing machinery into plants is to use Agrobacterium or biolistic transformation of plant tissue. In transformation, DNA coding for the editing machinery (e.g., CAS9 and guide RNA) is introduced into plant callus, seed or embryonic tissue. Stably-transformed plants (“events”) are then recovered, optionally with the help of a selectable marker. But because tissue culture is genotype-dependent, this route will not work for all crops, or even all varieties of the crops for which it does work. These are known as transformation-recalcitrant crops or varieties. These crops or varieties may be valued for their performance but it is a challenge for biotechnology that they cannot be transformed and thus cannot be directly edited via transformation. For recalcitrant varieties, one of two alternative approaches could be used to introduce desirable mutations. First, one could introduce the edits via trait introgression. This route is expensive, laborious, and time-consuming. It also means impurity of the final product because of genetic linkage—that is, there will be a linked block surrounding the introgressed edits, containing genes and alleles from the transformable donor line. This linkage can be an issue if any of those genes or alleles impact the performance of the transformation-recalcitrant line (may also be referred to as an “elite line”). Secondly, one could introduce the editing machinery transiently to the growing plant without tissue culture, such as floral dipping for Arabidopsis transformation. The challenge is ensuring edits end up in cells that contribute to the germ-line, so they are passed on to progeny seed. There are few established or routine methods to do this in crops.

Here we show a new method to transiently introduce editing machinery during haploid induction. Haploid induction (“HI”) is a class of plant phenomena characterized by loss of one parent's set of chromosomes (the chromosomes from the haploid inducer parent) from the embryo at some time during or after fertilization, often during early embryo development. Haploid induction is also known as gynogenesis if the inducer line is used as the male in the cross, or androgenesis if the inducer line is used as the female in the cross. Haploid induction has been observed in numerous plant species, such as sorghum, barley, wheat, maize, Arabidopsis, and many other species.

Commonly, during haploid induction, both parent lines used in the induction cross are both diploids, so their gametes (egg cells and sperm cells) are haploids. Haploid induction is frequently a medium to low penetrance trait of the inducer line, so the resulting progeny, depending on the species or situation, may be either diploid (if no genome loss takes place) or haploids (if genome loss does indeed take place). If the parent line that is crossed to the haploid inducer is not diploid, but rather a tetraploid, hexaploid, or other plant of higher ploidy, the term haploid induction is something of a misnomer, because the “haploid” progeny produced will have a gametic chromosome number, and thus would not really be haploids, but rather diploids (if the parent is tetraploid) or triploids (if the parent is hexaploid) and so on. Therefore, as used herein, “haploids” possess half the number of chromosomes of either parent; thus haploids of diploid organisms (e.g., maize) exhibit monoploidy; haploids of tetraploid organisms (e.g., ryegrasses) exhibit diploidy; haploids of hexaploid organisms (e.g., wheat) exhibit triploidy.

Haploid induction can occur during self-pollination or intercrossing of two lines within the same species, or it can occur during wide crosses, where it can be viewed as a hybridization barrier, preventing the formation of interspecific hybrids. In maize, the most commonly employed method of inducing haploids is through the use of an intraspecific haploid inducer male line, which is primarily triggered by rearrangements of, mutations in, and/or recombinations, insertion, or deletions within a region of chromosome 1, specifically the MATRILINEAL (MATL) gene, also known as NOT LIKE DAD1 (NLD1) and PHOSPHOLIPASE A1 (PLA1) (with the notable exception of the ig type haploid induction, which is a result of a mutation in the INDETERMINATE GAMETOPHYTE1 gene on chromosome 3). In wheat, the most common method of inducting haploids is by wide cross to maize pollen—regardless of parent genotype or lineage, this works with almost any wheat crossed by almost any maize pollen.

HI maize lines contain a quantitative trait locus (“QTL”) on Chromosome 1 responsible for at least 66% of the variation in haploid induction. The QTL causes haploid induction at different rates when it is introgressed into various backgrounds. All maize haploid inducer lines used in the seed industry are derivatives of the founding HI line, known as Stock6, and all have the haploid inducer chromosome 1 QTL mutation.

In maize, haploid seed or embryos are specifically produced by making crosses between a haploid inducer male (i.e., “haploid inducer pollen”) and virtually any ear that one chooses—the ear could be of any inbred, hybrid, or other germplasm. Haploids are produced when the haploid inducer pollen DNA is not fully transmitted and/or maintained through the first cell divisions of the embryos. The resulting phenotype is not fully penetrant, with some ovules containing haploid embryos, and others containing diploid embryos, aneuploid embryos, chimeric embryos, or aborted embryos. The haploid kernels have embryos that contain only the maternal DNA plus normal triploid endosperm. After haploid induction, haploid embryos or seed are typically segregated from diploid and aneuploid siblings using a phenotypic or genetic marker screen and grown or cultured into haploid plants. These plants are then converted either naturally or via chemical manipulation (e.g., using an anti-microtubule agent such as colchicine) into doubled haploid (“DH”) plants which then produce inbred seed.

Plant breeding is facilitated by the use of doubled haploid (DH) plants. The production of DH plants enables plant breeders to obtain inbred lines without multigenerational inbreeding, thus decreasing the time required to produce homozygous plants. DH plants provide an invaluable tool to plant breeders, particularly for generating inbred lines, QTL mapping, cytoplasmic conversions, trait introgression, and F2 screening for high throughput trait improvement. A great deal of time is spared as homozygous lines are essentially generated in one generation, negating the need for multigenerational single-seed decent (conventional inbreeding). In particular, because DH plants are entirely homozygous, they are very amenable to quantitative genetics studies. The production of haploid seed is critical for the doubled haploid breeding process. Haploid seed are produced on maternal germplasm when fertilized with pollen from a gynogenetic inducer, such as Stock 6 and Stock 6-derivative lines.

Here, we describe a novel method in which the in vivo haploid induction process can be co-opted to transiently introduce editing machinery into any germplasm by including it in the haploid inducer parent, either stably integrated as a transgene, or transiently expressed. Simultaneous editing plus haploid induction can be done in almost any crop via wide cross or de novo haploid induction for instance via CENH3 mutation (i.e., CENH3-modified haploid inducer; see, e.g., WO 2017/004375, incorporated herein by reference in its entirety) or via lipid spray (see P.C.T. Patent Application No. PCT/US2016/62548, incorporated herein by reference in its entirety). We show examples of HI in maize, both field corn and sweet corn, using a haploid inducer male as the editing donor line. Further, we show examples of HI in Arabidopsis using CENH3-modified haploid inducer lines.

We also show examples of HI in wheat using maize pollen as the editing donor line in a wide cross. In wheat, rice, barley, brassica, and other crops, the route to haploid induction would be to use a pollen donor that induces haploids via wide cross. For example, one could use corn pollen on wheat, millet pollen on wheat, barley pollen on other barley species, or any other wide crossing method. In those cases of gynogenetic haploid induction it would be preferable for the male line to contain the editing machinery, because it is the male (pollen-derived) DNA that is eliminated in the haploid induction process. In cases of androgenic haploid induction, for instance in the ig1 system in maize or via altered CENH3 in any crop (which can work via either the male or the female), the editing machinery would be optimally present in the female parent, because the female chromosomes are eliminated in the haploid induction process.

In simultaneous editing plus haploid induction, the goal is to rapidly and cost-effectively edit crops and elite lines (“editing destination lines”) without tissue culture. The line that receives the edits could be elite germplasm, and the editing machinery itself would be eliminated during the haploid induction process. At the same time, edited doubled haploid lines are produced.

### SUMMARY

Tissue culture recalcitrance is a major challenge to rapid elite line editing across crops. Using haploid inducing lines to deliver the targeted mutagenesis machinery to elite lines and simultaneously induce haploids represents the surmounting of this major obstacle. Next-generation breeding programs may come to depend on this process.

The editing machinery is delivered via the inducer line. The editing machinery is most often DNA-binding proteins combined in some cases with RNA and in some cases also with DNA. The DNA, RNA, and proteins that make up the editing machinery are encoded by and are present in the inducer line because they have been stably inserted in the inducer, for example, via bombardment or Agrobacterium mediated transformation. In other examples, the editing machinery is transiently introduced (through exogenous application) or transiently expressed in the gametophyte prior to fertilization. After fertilization, edits are made by the editing machinery in the non-inducer target genes prior to or during elimination of the inducer chromosomes. The result is a haploid embryo or plant or seed that contains the chromosome set only from the non-inducer parent, where that chromosome set contains DNA sequences that have been edited. These edited haploids can be identified, grown, and their chromosomes doubled, preferably by colchicine or other mitotic inhibitor. This line can then be directly used in downstream breeding programs.

In one embodiment, the invention provides a method of editing a plant's genomic DNA. This is done by taking a first plant—which is a haploid inducing plant and which also has encoded into its DNA the machinery necessary for accomplishing the editing (for example, a Cas9 enzyme and a guide RNA)—and using that first plant's pollen to pollinate a second plant. The second plant is the plant to be edited. From that pollination event, progeny (e.g., embryos or seeds) are produced; at least one of which will be a haploid seed. This haploid seed will only contain the chromosomes of the second plant; the first plant's chromosomes have vanished (having been eliminated, lost or degraded), but before doing so, the first plant's chromosomes permitted the gene-editing machinery to be expressed. Alternately, and without wishing to be bound by theory, the first plant delivers the already-expressed editing machinery upon pollination via the pollen tube. Or, in the case that the haploid inducer line is the female in the cross, the haploid inducing plant's egg cell contains the editing machinery that is present and perhaps already being expressed, upon fertilization with the “wild type” or non-haploid inducing pollen grain. Through any of these routes, the haploid progeny obtained by the cross will also have had its genome edited.

In one aspect, the editing machinery is any DNA modification enzyme, but is preferably a site-directed nuclease. The site-directed nuclease is preferably CRISPR-based, but could also be a meganuclease, a transcription-activator like effector nuclease (TALEN), or a zinc finger nuclease. The nuclease used in this invention could be Cas9, Cfp1, dCas9-FokI, chimeric FEN1-FokI. In one aspect, the DNA modification enzyme is a site-directed base editing enzyme such as Cas9-cytidine deaminase or Cas9-adenine deaminase, wherein the Cas9 can have one or both of its nuclease activity inactivated, i.e. chimeric Cas9 nickase (nCas9) or deactivated Cas9 (dCas9) fused to cytidine deaminase or adenine deaminase. The optional guide RNA targets the genome at the specific site intended to be edited. In one aspect, the optional guide RNA comprises an 18-21 nucleotide sequence with homology to any of SEQ ID NOs: 2, 4, 8, 21, 23, 25, 29, 32, and 33.

Once the edited haploid progeny is obtained, it may optionally have its chromosomes doubled by a chromosome doubling agent (for example colchicine, pronamide, dithipyr, trifluralin, or another known anti-microtubule agent).

In one embodiment, the first plant is a monocot or a dicot. Aspects of the first plant include maize, wheat, rice, barley, oats, triticale, sorghum, pearl millet, teosinte, bamboo, sugar cane, asparagus, onion, and garlic. In one embodiment, the second plant is a monocot or a dicot. Aspects of the second plant include maize, wheat, rice, barley, oats, triticale, sorghum, pearl millet, teosinte, bamboo, sugar cane, asparagus, onion, and garlic. In one embodiment, the first plant is a monocot or a dicot of a different species than the second plant. For example, in one aspect, the first plant is maize and the second plant is wheat. In another aspect, the first plant is wheat and the second plant is maize. In another embodiment, the first plant is a maize plant selected and/or derived from the lines Stock 6, RWK, RWS, UH400, AX5707RS, NP2222-matl, or any of the several other known HI lines. In yet another embodiment, the first plant comprises a mutation in a CENH3 gene, an ig1 gene, or another mutation conferring paternal-haploid inducing systems. In another embodiment, the first plant is a rice plant with the MATL gene modified or knocked out which makes it a haploid inducer line.

In another embodiment, the first plant is not necessarily a haploid inducer, yet the first plant comprises the genes necessary for encoding the gene editing machinery. In this embodiment, haploid induction is produced by administering a compound during, immediately before, or immediately following pollination. In one aspect, the composition comprises a lipid or a phospholipase inhibitor. In another aspect, the composition comprises methyl alpha-linolenoyl fluorophosphonate (MALFP), linoleic acid ethyl ester (LLAEE), linoleic acid (LLA), corn oil, distearoyl-phosphatidyl choline (DSPC), methyl arachidonyl fluorophosphonate (MAFP), Palmityl trifluoromethylketone (PACOCF3), Arachidonyl trifluoromethylketone (AACOCF3), Manoalide, Linolenic acid ethyl ester (LNAEE), Linolenic acid ethyl ester (LNAEE), Oleic acid methyl ester (OAME), Oleic acid ethyl ester (OAEE), Palmitic acid ethyl ester (PAEE), Palmitoleic acid ethyl ester (PLAEE), Linseed oil, corn oil, alpha-Linolenic acid (aLNA), gamma-Linolenic acid (gLNA), Oleic acid, Arachidonic acid, Stearic Acid, 9(Z)-11(E)-conjugated Linoleic acid, or 2-oleoyl-1-palmitoyl-sn-glycero-3-phospho-ethanolamine.

### BRIEF DESCRIPTION OF THE SEQUENCES IN THE SEQUENCE LISTING

SEQ ID NO: 1 is a nucleotide sequence for vector 23396.

SEQ ID NO: 2 is the nucleotide sequence encoding the gRNA sequence for editing VLHP1 in maize.

SEQ ID NO: 3 is a nucleotide sequence for vector 23399.

SEQ ID NO: 4 is the gRNA sequence for editing GW2-2 in maize.

SEQ ID NO: 5 is the nucleotide sequence for vector 22808, comprising a TALEN construct.

SEQ ID NO: 6 is the target sequence for the TALEN of 22808.

SEQ ID NO: 7 is the nucleotide sequence for vector 23123 comprising a Cas9 construct.

SEQ ID NO: 8 is the gRNA for editing MATL in maize.

SEQ ID NO: 9 is nucleotide sequence for the relevant portion of MATL in NP2222.

SEQ ID NO: 10 is nucleotide sequence for the relevant portion of MATL in Stock6.

SEQ ID NO: 11 is nucleotide sequence for the relevant portion of MATL in USR01350333-3 Allele 1.

SEQ ID NO: 12 is nucleotide sequence for the relevant portion of MATL in USR01350333-3 Allele 2.

SEQ ID NO: 13 is nucleotide sequence for the relevant portion of MATL in USR01350344-2 Allele 1.

SEQ ID NO: 14 is nucleotide sequence for the relevant portion of MATL in USR01350344-2 Allele 2.

SEQ ID NO: 15 is nucleotide sequence for the relevant portion of MATL in USR01350343-1 Allele 1.

SEQ ID NO: 16 is nucleotide sequence for the relevant portion of MATL in USR01350328-1 Allele 1.

SEQ ID NO: 17 is nucleotide sequence for the relevant portion of MATL in USR01350337-2 Allele 1.

SEQ ID NO: 18 is nucleotide sequence for the relevant portion of MATL in USR01350337-2 Allele 2.

SEQ ID NO: 19 is the nucleotide sequence of cDNA wildtype MATL.

SEQ ID NO: 20 is the nucleotide sequence for vector 23397.

SEQ ID NO: 21 is the gRNA sequence for editing VLHP2 in maize.

SEQ ID NO: 22 is the nucleotide sequence for vector 23398.

SEQ ID NO: 23 is the gRNA sequence for editing GW2-1 in maize.

SEQ ID NO: 24 is the nucleotide sequence for vector 23763.

SEQ ID NO: 25 is the gRNA sequence for VLHP1 in wheat.

SEQ ID NO: 26 is the wheat VLHP target sequence for TaVLHP2.

SEQ ID NO: 27 is the wheat VLHP target sequence for TaVLHP3.

SEQ ID NO: 28 is the target sequence in ZmVLHP2-03 for editing.

SEQ ID NO: 29 is the edited sequence in ZmVLHP2-03.

SEQ ID NO: 30 is the repair donor template sequence for creating E149L mutation in ZmPYL-D.

SEQ ID NO: 31 is the nucleotide sequence for vector 23136.

SEQ ID NO: 32 is the gRNA of vector 23136.

SEQ ID NO: 33 is the nucleotide sequence of rice PLA gene Os03g27610.

SEQ ID NO: 34 is the nucleotide sequence for vector 24038.

SEQ ID NO: 35 is the nucleotide sequence for vector 24039.

SEQ ID NO: 36 is the nucleotide sequence for vector 24079.

SEQ ID NO: 37 is the nucleotide sequence for vector 24091.

SEQ ID NO: 38 is the nucleotide sequence for vector 24094.

SEQ ID NOs: 39 through 97 are primers and probes used in the identified PCR Taqman assays.

SEQ ID NO: 98 is the nucleotide sequence for vector 24075.

SEQ ID NO: 99 is a portion of the edited GW2-02 target site in haploid sweet corn line JSER82A063, shown in FIG. 13.

SEQ ID NO: 100 is the reverse complement of SEQ ID NO: 99 shown in FIG. 13.

SEQ ID NO: 101 is a portion of the edited TaVLHP1-4B target site in haploid wheat line JSWER30A22, shown in FIG. 16.

SEQ ID NO: 102 is the nucleotide sequence of the gRNA used in editing the Arabidopsis GL1 gene.

SEQ ID NO: 103 is the relevant portion of the wildtype Arabidopsis GL1 gene.

SEQ ID NO: 104 is the relevant portion of the edited GL1 gene (by single nucleotide deletion) in individual 135.

SEQ ID NO: 105 is the relevant portion of the edited GL1 gene (by single nucleotide insertion) in individual 135.

SEQ ID NO: 106 is the relevant portion of the unedited GL1 gene in individual 1033-A3 (product of cross between USR01424135 and Ler-425).

SEQ ID NO: 107 is the relevant portion of the edited GL1 gene (by single nucleotide insertion) in individual 1033-C3 (product of cross between USR01424135 and Ler-427).

SEQ ID NO: 108 is the relevant portion of the edited GL1 gene (by single nucleotide insertion) in individual 1033-E4 (product of cross between USR01424135 and Ler-437).

SEQ ID NO: 109 is the relevant portion of the edited GL1 gene (by deletion of three nucleotides) in individual 1041-H12.

SEQ ID NO: 110 is the relevant portion of the edited GL1 gene (by single nucleotide insertion) in individual 1042-E5 (product of cross between USR01424136 and Ler-25).

SEQ ID NO: 111 is the relevant portion of the edited GL1 gene (by single nucleotide deletion) in individual 1042-G12 (product of cross between USR01424136 and Ler-83).

SEQ ID NO: 112 is the relevant portion of the edited GL1 gene (by deletion of two nucleotides) in individual 1042-G10 (product of cross between USR01424136 and Ler-67).

SEQ ID NO: 113 is the relevant portion of the edited GL1 gene (by deletion of two nucleotides) in individual 1045-E3 (product of cross between USR01424136 and Ler-261).

SEQ ID NO: 114 is the relevant portion of the edited GL1 gene (by single nucleotide insertion) in individual 1045-D3 (product of cross between USR01424136 and Ler-260).

SEQ ID NO: 115 is the relevant portion of the edited GL1 gene (by single nucleotide insertion) in individual 1046-D11 (product of cross between USR01431609 and Ler-111).

SEQ ID NO: 116 is the relevant portion of the edited GL1 gene (by single nucleotide insertion) in individual 1046-G12 (product of cross between USR01431609 and Ler-122).

SEQ ID NO: 117 is the relevant portion of the edited GL1 gene (by deletion of sixteen nucleotides and insertion of eight nucleotides) in individual 1045-F2 (product of cross between USR01424136 and Ler-254).

### BRIEF DESCRIPTION OF THE FIGURES

FIG. 1 shows the ploidy analysis (flow cytometry) data for USR01350334-3: DIPLOID (major peak at 200, secondary peak at 400).

FIG. 2 shows the ploidy analysis (flow cytometry) data for USR01350333-3: HAPLOID (major peak at 100, secondary peak at 200).

FIG. 3 shows the ploidy analysis (flow cytometry) data for USR01350333-10: DIPLOID (major peak at 200, secondary peak at 400).

FIG. 4 shows the ploidy analysis (flow cytometry) data for USR01350344-2: HAPLOID (major peak at 100, secondary peak at 200).

FIG. 5 shows the ploidy analysis (flow cytometry) data for USR01350343-1: HAPLOID (major peak at 100, secondary peak at 200).

FIG. 6 shows the ploidy analysis (flow cytometry) data for USR01350341-1: DIPLOID (major peak at 200, secondary peak at 400).

FIG. 7 shows the ploidy analysis (flow cytometry) data for USR01350328-1: HAPLOID (major peak at 100, secondary peak at 200).

FIG. 8 shows the ploidy analysis (flow cytometry) data for USR01350321-3: DIPLOID (major peak at 200, secondary peak at 400).

FIG. 9 is a schematic drawing of vector 23396 (SEQ ID NO: 1) used for Agrobacterium-mediated transformation of maize immature embryos to generate targeted mutations in ZmVLHP1 genes. xZmVLHP-01: guide RNA (gRNA) sequence (5′-GCAGGAGGCGTCGAGCAGCG-3′, SEQ ID NO: 2); rsgRNAZmVLHP-01: single guide RNA (sgRNA) comprising of gRNA, tracRNA and PolIII termination sequences. cPMI: PMI selectable marker gene; cCas9: Cas9 nuclease gene; RB: T-DNA right border; LB: T-DNA left border; tNOS: Nopaline synthetase terminator. cSpec: Spectinomycin resistance gene.

FIG. 10 is a schematic drawing of vector 23399 (SEQ ID NO: 3) used for Agrobacterium-mediated transformation of maize immature embryos to generate targeted mutations in ZmGW2 genes. xZmGW2-02: guide RNA (gRNA) sequence (5′-AAGCTCGCGCCCTGCTACCC-3′, SEQ ID NO: 4); rsgRNAZmGW2-02: single guide RNA (sgRNA) comprising of gRNA, tracrRNA and PolIII termination sequences. cPMI-09: PMI selectable marker gene; cCas9-01: Cas9 nuclease gene; RB: T-DNA right border; LB: T-DNA left border; tNOS: Nopaline synthetase terminator. cSpec: Spectinomycin resistance gene.

FIG. 11 shows ploidy assay of edited haploid sweet corn line JSER82A056 and FIG. 12 shows the same for edited haploid sweet corn line JSER82A063. These lines were obtained through crossing with RWKS haploid induction line carrying transgene locus of CRISPR-Cas9 expression vector 23399.

FIG. 13 shows sequencing confirmation of GW2-02 target site editing in haploid sweet corn line JSER82A063. A single base C next to the predicted Cas9 cleavage site was deleted. The sequence presented within the box is identical to SEQ ID NO: 4. The top-line sequence presented at the bottom of the figure is represented by SEQ ID NO: 99. The bottom-line sequence is represented by SEQ ID NO: 100 and is the reverse complement of SEQ ID NO: 99.

FIG. 14 shows ploidy analysis of wild type control, and FIG. 15 shows ploidy analysis of edited haploid wheat line JSWER30A22.

FIG. 16 shows sequencing confirmation of TaVLHP1-4B target site editing in haploid wheat line JSWER30A22. Lower panel showing 97 bp of TaVLHP1-4B sequence was deleted immediately downstream of the predicted Cas9 cleavage site. The 97 bp deleted sequences were marked by 2 arrows. The underlined sequence matches the gRNA sequence of SEQ ID NO: 25. The entire sequence is represented by SEQ ID NO: 101.

FIG. 17 shows pollen expression as measured by pollen collected from transgenic maize T0 plants carrying T-DNA of vector 24038, 24039, 24079, 24091, and 24094, which were used to pollinate emasculated spring wheat line AC-Nanda. The expression was high in the pollen, averaging about 100 fold higher in plants carrying T-DNA vectors 24038, 24039, and 24079 compared to the sugar cane ubiquitin promoter used in many of the corn and wheat examples. The expression was also higher in pollen from plants containing vectors 24038, 24039, and 24079 when compared to the pollen carrying the MATRILINEAL promoter constructs (24094 and 24091).

FIG. 18 shows the ploidy analysis histogram of a diploid control (parent USR01424135). The first peak is located at the “2” position on the logarithmic X-axis. Because this is leaf tissue from Arabidopsis, we see multiple peaks indicating the normal level of endoreduplication.

FIG. 19 shows the ploidy analysis histogram of a diploid control (parent USR01431603). The first peak is located at the “2” position on the logarithmic X-axis. Because this is leaf tissue from Arabidopsis, we see multiple peaks indicating the normal level of endoreduplication.

FIG. 20 shows the ploidy analysis histogram of a diploid control (parent USR01431609). The first peak is located at the “2” position on the logarithmic X-axis. Because this is leaf tissue from Arabidopsis, we see multiple peaks indicating the normal level of endoreduplication.

FIG. 21 shows the ploidy analysis histogram of an edited haploid from plate 1033, well C3 (USR01424135 X Ler-427). The first peak is located at the “1” position on the logarithmic X-axis, indicating it is a haploid. Because this is leaf tissue from Arabidopsis, we see multiple peaks indicating the normal level of endoreduplication.

FIG. 22 shows the ploidy analysis histogram of an edited haploid from plate 1033, well E4 (USR01424135 X Ler-437). The first peak is located at the “1” position on the logarithmic X-axis, indicating it is a haploid. Because this is leaf tissue from Arabidopsis, we see multiple peaks indicating the normal level of endoreduplication.

FIG. 23 shows the ploidy analysis histogram of an edited haploid from plate 1046, well H12 (USR01431609 X Ler-123). The first peak is located at the “1” position on the logarithmic X-axis, indicating it is a haploid. Because this is leaf tissue from Arabidopsis, we see multiple peaks indicating the normal level of endoreduplication.

FIG. 24 shows the GL1 target site sequence mutations in the parent #USR01424135 and all of the sequenced edited haploids from outcrosses by Landsberg erecta pollen. It is clear that the precise edit made is different in the different haploids. From top to bottom, the sequences shown are represented by SEQ ID NOs: 102-117, respectively.

### DEFINITIONS

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a,” “an,” and “the” refer to “one or more” when used in this application, including the claims. For example, the phrase “a cell” refers to one or more cells, and in some embodiments can refer to a tissue and/or an organ. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to all whole number values between 1 and 100 as well as whole numbers greater than 100.

Unless otherwise indicated, all numbers expressing quantities of ingredients, reaction conditions, and so forth used in the specification and claims are to be understood as being modified in all instances by the term “about.” The term “about,” as used herein when referring to a measurable value such as an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the discloses compositions, nucleic acids, polypeptides, etc. Accordingly, unless indicated to the contrary, the numerical parameters set forth in this specification and attached claims are approximations that can vary depending upon the desired properties sought to be obtained by the presently disclosed subject matter.

As used herein, the term “allele” refers to a variant or an alternative sequence form at a genetic locus. In diploids, a single allele is inherited by a progeny individual separately from each parent at each locus. The two alleles of a given locus present in a diploid organism occupy corresponding places on a pair of homologous chromosomes, although one of ordinary skill in the art understands that the alleles in any particular individual do not necessarily represent all of the alleles that are present in the species.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D (e.g., AB, AC, AD, BC, BD, CD, ABC, ABD, and BCD). In some embodiments, one of more of the elements to which the “and/or” refers can also individually be present in single or multiple occurrences in the combinations(s) and/or subcombination(s).

As used herein, the phrase “associated with” refers to a recognizable and/or assayable relationship between two entities. For example, the phrase “associated with HI” refers to a trait, locus, gene, allele, marker, phenotype, etc., or the expression thereof, the presence or absence of which can influence an extent and/or degree at which a plant or its progeny exhibits HI. As such, a marker is “associated with” a trait when it is linked to it and when the presence of the marker is an indicator of whether and/or to what extent the desired trait or trait form will occur in a plant/germplasm comprising the marker. Similarly, a marker is “associated with” an allele when it is linked to it and when the presence of the marker is an indicator of whether the allele is present in a plant/germplasm comprising the marker. For example, “a marker associated with HI” refers to a marker whose presence or absence can be used to predict whether and/or to what extent a plant will display haploid induction.

The term “comprising,” which is synonymous with “including,” “containing,” and “characterized by,” is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter.

With respect to the terms “comprising,” “consisting essentially of,” and “consisting of,” where one of these three terms is used herein, the presently disclosed and claimed subject matter can include in some embodiments the use of either of the other two terms. For example, if a subject matter relates in some embodiments to nucleic acids that encode polypeptides comprising amino acid sequences that are at least 95% identical to a SEQ ID NO: 9 or 19. It is understood that the disclosed subject matter thus also encompasses nucleic acids that encode polypeptides that in some embodiments consist essentially of amino acid sequences that are at least 95% identical to that SEQ ID NO: 9 or 19 as well as nucleic acids that encode polypeptides that in some embodiments consist of amino acid sequences that are at least 95% identical to that SEQ ID NO: 9 or 19. Similarly, it is also understood that in some embodiments the methods for the disclosed subject matter comprise the steps that are disclosed herein, in some embodiments the methods for the presently disclosed subject matter consist essentially of the steps that are disclosed, and in some embodiments the methods for the presently disclosed subject matter consist of the steps that are disclosed herein.

As used herein, the term “de novo haploid induction” refers to the triggering of haploid induction by the introduction of a spontaneous haploid inducing agent. Such introduction can be achieved by topical spray, hand-pollination, mutagenesis, or transgenic methods. The terms “de novo haploid induction,” “de novo HI,” and “haploid induction de novo” are used interchangeably throughout this specification.

As used herein, the term “gene” refers to a hereditary unit including a sequence of DNA that occupies a specific location on a chromosome and that contains the genetic instruction for a particular characteristic or trait in an organism.

A “genetic map” is a description of genetic linkage relationships among loci on one or more chromosomes within a given species, generally depicted in a diagrammatic or tabular form.

As used herein, a plant referred to as “haploid” has a reduced number of chromosomes (n) in the haploid plant, and its chromosome set is equal to that of the gamete. In a haploid organism, only half of the normal number of chromosomes are present. Thus haploids of diploid organisms (e.g., maize) exhibit monoploidy; haploids of tetraploid organisms (e.g., ryegrasses) exhibit diploidy; haploids of hexaploid organisms (e.g., wheat) exhibit triploidy; etc. As used herein, a plant referred to as “doubled haploid” is developed by doubling the haploid set of chromosomes. A plant or seed that is obtained from a doubled haploid plant that is selfed to any number of generations may still be identified as a doubled haploid plant. A doubled haploid plant is considered a homozygous plant. A plant is considered to be doubled haploid if it is fertile, even if the entire vegetative part of the plant does not consist of the cells with the doubled set of chromosomes; that is, a plant will be considered doubled haploid if it contains viable gametes, even if it is chimeric in vegetative tissues.

As used herein, the term “human-induced mutation” refers to any mutation that occurs as a result of either direct or indirect human action. This term includes, but is not limited to, mutations obtained by any method of targeted mutagenesis.

As used herein, “introduced” means delivered, expressed, applied, transported, transferred, permeated, or other like term to indicate the delivery, whether of nucleic acid or protein or combination thereof, of a desired object to an object. For example, nucleic acids encoding a site directed nuclease and optionally at least one guide RNA may be introduced into a haploid embryo upon haploid induction. Likewise, extant editing machinery (comprising a site directed nuclease protein and optionally at least one guide RNA) may be introduced to a haploid embryo upon application of appropriate cell-penetrating peptides.

As used herein, the terms “marker probe” and “probe” refer to a nucleotide sequence or nucleic acid molecule that can be used to detect the presence or absence of a sequence within a larger sequence, e.g., a nucleic acid probe that is complementary to all of or a portion of the marker or marker locus, through nucleic acid hybridization. Marker probes comprising about 8, 10, 15, 20, 30, 40, 50, 60, 70, 80, 90, 100 or more contiguous nucleotides can be used for nucleic acid hybridization.

As used herein, the term “molecular marker” can be used to refer to a genetic marker, as defined above, or an encoded product thereof (e.g., a protein) used as a point of reference when identifying the presence/absence of a HI-associated locus. A molecular marker can be derived from genomic nucleotide sequences or from expressed nucleotide sequences (e.g., from an RNA, a cDNA, etc.). The term also refers to nucleotide sequences complementary to or flanking the marker sequences, such as nucleotide sequences used as probes and/or primers capable of amplifying the marker sequence. Nucleotide sequences are “complementary” when they specifically hybridize in solution (e.g., according to Watson-Crick base pairing rules). This term also refers to the genetic markers that indicate a trait by the absence of the nucleotide sequences complementary to or flanking the marker sequences, such as nucleotide sequences used as probes and/or primers capable of amplifying the marker sequence.

As used herein, the terms “nucleotide sequence,” “polynucleotide,” “nucleic acid sequence,” “nucleic acid molecule,” and “nucleic acid fragment” refer to a polymer of RNA or DNA that is single- or double-stranded, optionally containing synthetic, non-natural, and/or altered nucleotide bases. A “nucleotide” is a monomeric unit from which DNA or RNA polymers are constructed and consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

As used herein, the term “nucleotide sequence identity” refers to the presence of identical nucleotides at corresponding positions of two polynucleotides. Polynucleotides have “identical” sequences if the sequence of nucleotides in the two polynucleotides is the same when aligned for maximum correspondence (e.g., in a comparison window). Sequence comparison between two or more polynucleotides is generally performed by comparing portions of the two sequences over a comparison window to identify and compare local regions of sequence similarity. The comparison window is generally from about 20 to 200 contiguous nucleotides. The “percentage of sequence identity” for polynucleotides, such as about 50, 55, 60, 65, 70, 75, 80, 85, 90, 95, 98, 99 or 100 percent sequence identity, can be determined by comparing two optimally aligned sequences over a comparison window, wherein the portion of the polynucleotide sequence in the comparison window can include additions or deletions (i.e., gaps) as compared to the reference sequence for optimal alignment of the two sequences. In some embodiments, the percentage is calculated by: (a) determining the number of positions at which the identical nucleic acid base occurs in both sequences; (b) dividing the number of matched positions by the total number of positions in the window of comparison; and (c) multiplying the result by 100. Optimal alignment of sequences for comparison can also be conducted by computerized implementations of known algorithms, or by visual inspection. Readily available sequence comparison and multiple sequence alignment algorithms are, respectively, the Basic Local Alignment Search Tool (BLAST) and ClustalW/ClustalW2/Clustal Omega programs available on the Internet (e.g., the website of the EMBL-EBI). Other suitable programs include, but are not limited to, GAP, BestFit, Plot Similarity, and FASTA, which are part of the Accelrys GCG Package available from Accelrys, Inc. of San Diego, California, United States of America. See also Smith & Waterman, 1981; Needleman & Wunsch, 1970; Pearson & Lipman, 1988; Ausubel et al., 1988; and Sambrook & Russell, 2001.

One example of an algorithm that is suitable for determining percent sequence identity and sequence similarity is the BLAST algorithm, which is described in Altschul et al., 1990. In some embodiments, a percentage of sequence identity refers to sequence identity over the full length of one of the gDNA, cDNA, or the predicted protein sequences in the largest ORF of SEQ ID No: 1 being compared. In some embodiments, a calculation to determine a percentage of nucleic acid sequence identity does not include in the calculation any nucleotide positions in which either of the compared nucleic acids includes an “N” (i.e., where any nucleotide could be present at that position).

The term “open reading frame” (ORF) refers to a nucleic acid sequence that encodes a polypeptide. In some embodiments, an ORF comprises a translation initiation codon (i.e., start codon), a translation termination (i.e., stop codon), and the nucleic acid sequence there between that encodes the amino acids present in the polypeptide. The terms “initiation codon” and “termination codon” refer to a unit of three adjacent nucleotides (i.e., a codon) in a coding sequence that specifies initiation and chain termination, respectively, of protein synthesis (mRNA translation).

Patatin-like phospholipase A2α may also be known as PLA, pPLA, pPLAIIA pPLAIIα, PLA2alpha, or PLA2, or other similar variation. Patatin-like phospholipase AIIα is also referred to as MATRILINEAL (MATL). These terms are used interchangeably throughout. A MATRILINEAL gene comprising a four basepair frameshift mutation is referred to as matrilineal (matl).

As used herein, the terms “phenotype,” “phenotypic trait” or “trait” refer to one or more traits of a plant or plant cell. The phenotype can be observable to the naked eye, or by any other means of evaluation known in the art, e.g., microscopy, biochemical analysis, or an electromechanical assay. In some cases, a phenotype is directly controlled by a single gene or genetic locus (i.e., corresponds to a “single gene trait”). In the case of haploid induction use of color markers, such as R Navajo, and other markers including transgenes visualized by the presences or absences of color within the seed evidence if the seed is an induced haploid seed. The use of R Navajo as a color marker and the use of transgenes is well known in the art as means to detect induction of haploid seed on the female plant. In other cases, a phenotype is the result of interactions among several genes, which in some embodiments also results from an interaction of the plant and/or plant cell with its environment.

As used herein, the term “plant” can refer to a whole plant, any part thereof, or a cell or tissue culture derived from a plant. Thus, the term “plant” can refer to any of: whole plants, plant components or organs (e.g., leaves, stems, roots, etc.), plant tissues, seeds and/or plant cells.

A plant cell is a cell of a plant, taken from a plant, or derived through culture from a cell taken from a plant. Thus, the term “plant cell” includes without limitation cells within seeds, suspension cultures, embryos, meristematic regions, callus tissue, leaves, shoots, gametophytes, sporophytes, pollen, and microspores. The phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, calli, and the like.

As used herein, the term “primer” refers to an oligonucleotide which is capable of annealing to a nucleic acid target (in some embodiments, annealing specifically to a nucleic acid target) allowing a DNA polymerase and/or reverse transcriptase to attach thereto, thereby serving as a point of initiation of DNA synthesis when placed under conditions in which synthesis of a primer extension product is induced (e.g., in the presence of nucleotides and an agent for polymerization such as DNA polymerase and at a suitable temperature and pH). In some embodiments, one or more pluralities of primers are employed to amplify plant nucleic acids (e.g., using the polymerase chain reaction; PCR).

As used herein, the term “probe” refers to a nucleic acid (e.g., a single stranded nucleic acid or a strand of a double stranded or higher order nucleic acid, or a subsequence thereof) that can form a hydrogen-bonded duplex with a complementary sequence in a target nucleic acid sequence. Typically, a probe is of sufficient length to form a stable and sequence-specific duplex molecule with its complement, and as such can be employed in some embodiments to detect a sequence of interest present in a plurality of nucleic acids.

As used herein, the terms “progeny” and “progeny plant” refer to a plant generated from vegetative or sexual reproduction from one or more parent plants. In gynogenesis-mediated haploid induction, the haploid embryo on the female parent comprises female chromosomes to the exclusion of male chromosomes—thus it is not a progeny of the male haploid-inducing line. The haploid corn seed typically still has normal triploid endosperm that contains the male genome. The edited haploid progeny and subsequent edited doubled haploid plants and subsequent seed is not the only desired progeny. There is also the seed from the haploid inducer line itself, often carrying the Cas9 transgene, and subsequent plant and seed progeny of the haploid inducing plant. Both the haploid seed and the haploid inducer (self-pollination-derived) seed can be progeny. A progeny plant can be obtained by cloning or selfing a single parent plant, or by crossing two or more parental plants. For instance, a progeny plant can be obtained by cloning or selfing of a parent plant or by crossing two parental plants and include selfings as well as the F₁ or F₂ or still further generations. An F₁ is a first-generation progeny produced from parents at least one of which is used for the first time as donor of a trait, while progeny of second generation (F₂) or subsequent generations (F₃, F₄, and the like) are specimens produced from selfings, intercrosses, backcrosses, and/or other crosses of F₁s, F₂s, and the like. An F₁ can thus be (and in some embodiments is) a hybrid resulting from a cross between two true breeding parents (i.e., parents that are true-breeding are each homozygous for a trait of interest or an allele thereof), while an F₂ can be (and in some embodiments is) a progeny resulting from self-pollination of the F₁ hybrids.

As used herein, the phrase “recombination” refers to an exchange of DNA fragments between two DNA molecules or chromatids of paired chromosomes (a “crossover”) over in a region of similar or identical nucleotide sequences. A “recombination event” is herein understood to refer in some embodiments to a meiotic crossover.

As used herein, the term “reference sequence” refers to a defined nucleotide sequence used as a basis for nucleotide sequence comparison. In some embodiments, any of SEQ ID NOs: 2, 4, 8, 21, 23, 25, 29, 32, and 33 can serve as a reference sequence for comparing to other sequences obtained from plants.

As used herein, the term “regenerate,” and grammatical variants thereof, refers to the production of a plant from tissue culture.

As used herein, the phrase “stringent hybridization conditions” refers to conditions under which a polynucleotide hybridizes to its target subsequence, typically in a complex mixture of nucleic acids, but to essentially no other sequences. Stringent conditions are sequence-dependent and can be different under different circumstances.

Longer sequences typically hybridize specifically at higher temperatures. An extensive guide to the hybridization of nucleic acids is found in Sambrook & Russell, 2001. Generally, stringent conditions are selected to be about 5-10° C. lower than the thermal melting point (Tₘ) for the specific sequence at a defined ionic strength pH. The Tm is the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Exemplary stringent conditions are those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides).

Stringent conditions can also be achieved with the addition of destabilizing agents such as formamide. Additional exemplary stringent hybridization conditions include 50% formamide, 5×SSC, and 1% SDS incubating at 42° C.; or SSC, 1% SDS, incubating at 65° C.; with one or more washes in 0.2×SSC and 0.1% SDS at 65° C. For PCR, a temperature of about 36° C. is typical for low stringency amplification, although annealing temperatures can vary between about 32° C. and 48° C. (or higher) depending on primer length. Additional guidelines for determining hybridization parameters are provided in numerous references (see e.g., Ausubel et al., 1999).

As used herein, the term “trait” refers to a phenotype of interest, a gene that contributes to a phenotype of interest, as well as a nucleic acid sequence associated with a gene that contributes to a phenotype of interest. For example, a “HI trait” refers to a haploid induction phenotype as well as a gene (e.g., matl in maize or Os03g27610 in rice) that contributes to a haploid induction and a nucleic acid sequence (e.g., a HI-associated gene product) that is associated with the presence or absence of the haploid induction phenotype.

As used herein, the term “transgene” refers to a nucleic acid molecule introduced into an organism or one or more of its ancestors by some form of artificial transfer technique. The artificial transfer technique thus creates a “transgenic organism” or a “transgenic cell.” It is understood that the artificial transfer technique can occur in an ancestor organism (or a cell therein and/or that can develop into the ancestor organism) and yet any progeny individual that has the artificially transferred nucleic acid molecule or a fragment thereof is still considered transgenic even if one or more natural and/or assisted breedings result in the artificially transferred nucleic acid molecule being present in the progeny individual.

As used herein, the term “targeted mutagenesis” or “mutagenesis strategy” refers to any method of mutagenesis that results in the intentional mutagenesis of a chosen gene. Targeted mutagenesis includes the methods CRISPR, TILLING, TALEN, and other methods not yet discovered but which may be used to achieve the same outcome.

As used herein, haploid induction rate (“HIR”) means the number of surviving haploid kernels over the total number of kernels after an ear is pollinated with haploid inducer pollen.

Particular problems plague that haploid induction: increased embryo abortion rates and increased fertilization failure rates (reduced seed set rates). For these reasons, there exists a need to successfully determine the cause of HI, and to use that knowledge to determine methods of stably or increasingly creating haploid plants while simultaneously reducing fertilization failure and embryo abortions.

It is specifically contemplated that one could mutagenize a promoter to potentially improve the utility of the elements for the expression of transgenes in plants. The mutagenesis of these elements can be carried out at random and the mutagenized promoter sequences screened for activity in a trial-by-error procedure. Alternatively, particular sequences which provide the promoter with desirable expression characteristics, or the promoter with expression enhancement activity, could be identified and these or similar sequences introduced into the promoter via mutation. It is further contemplated that one could mutagenize these sequences in order to enhance their expression of transgenes in a particular species. The means for mutagenizing a DNA segment encoding a promoter sequence of the current invention are well-known to those of skill in the art. As indicated, modifications to promoter or other regulatory element may be made by random, or site-specific mutagenesis procedures. The promoter and other regulatory element may be modified by altering their structure through the addition or deletion of one or more nucleotides from the sequence which encodes the corresponding unmodified sequences.

Mutagenesis may be performed in accordance with any of the techniques known in the art, such as, and not limited to, synthesizing an oligonucleotide having one or more mutations within the sequence of a particular regulatory sequence. In particular, site-specific mutagenesis is a technique useful in the preparation of promoter mutants, through specific mutagenesis of the underlying DNA. RNA-guided endonucleases (“RGEN,” e.g., CRISPR/Cas9) may also be used. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered.

Where a clone comprising a promoter has been isolated in accordance with the instant invention, one may wish to delimit the essential promoter regions within the clone. One efficient, targeted means for preparing mutagenized promoters relies upon the identification of putative regulatory elements within the promoter sequence. This can be initiated by comparison with promoter sequences known to be expressed in similar tissue specific or developmentally unique patterns. Sequences which are shared among promoters with similar expression patterns are likely candidates for the binding of transcription factors and are thus likely elements which confer expression patterns. Confirmation of these putative regulatory elements can be achieved by deletion analysis of each putative regulatory sequence followed by functional analysis of each deletion construct by assay of a reporter gene which is functionally attached to each construct. As such, once a starting promoter sequence is provided, any of a number of different deletion mutants of the starting promoter could be readily prepared.

The invention disclosed herein provides polynucleotide molecules comprising regulatory element fragments that may be used in constructing novel chimeric regulatory elements. Novel combinations comprising fragments of these polynucleotide molecules and at least one other regulatory element or fragment can be constructed and tested in plants and are considered to be within the scope of this invention. Thus the design, construction, and use of chimeric regulatory elements is one embodiment of this invention. Promoters of the present invention include homologues of cis elements known to affect gene regulation that show homology with the promoter sequences of the present invention.

Functional equivalent fragments of one of the transcription regulating nucleic acids described herein comprise at least 50, 100, 150, 200, 250, 300, 350, 400, 450, 500, 550, 600, 650, 700, 750, 800, 850, 900, 950, or 1000 base pairs of a transcription regulating nucleic acid. Equivalent fragments of transcription regulating nucleic acids, which are obtained by deleting the region encoding the 5′-untranslated region of the mRNA, would then only provide the (untranscribed) promoter region. The 5′-untranslated region can be easily determined by methods known in the art (such as 5′-RACE analysis). Accordingly, some of the transcription regulating nucleic acids, described herein, are equivalent fragments of other sequences.

As indicated above, deletion mutants of the promoter of the invention also could be randomly prepared and then assayed. Following this strategy, a series of constructs are prepared, each containing a different portion of the promoter (a subclone), and these constructs are then screened for activity. A suitable means for screening for activity is to attach a deleted promoter or intron construct which contains a deleted segment to a selectable or screenable marker, and to isolate only those cells expressing the marker gene. In this way, a number of different, deleted promoter constructs are identified which still retain the desired, or even enhanced, activity. The smallest segment which is required for activity is thereby identified through comparison of the selected constructs. This segment may then be used for the construction of vectors for the expression of exogenous genes.

An expression cassette as described herein may comprise further regulatory elements. The term in this context is to be understood in the broad meaning comprising all sequences which may influence construction or function of the expression cassette. Regulatory elements may, for example, modify transcription and/or translation in prokaryotic or eukaryotic organisms. The expression cassette described herein may be downstream (in 3′ direction) of the nucleic acid sequence to be expressed and optionally contain additional regulatory elements, such as transcriptional or translational enhancers. Each additional regulatory element may be operably liked to the nucleic acid sequence to be expressed (or the transcription regulating nucleotide sequence). Additional regulatory elements may comprise additional promoters, minimal promoters, promoter elements, or transposon elements which may modify or enhance the expression regulating properties. The expression cassette may also contain one or more introns, one or more exons and one or more terminators.

Furthermore, it is contemplated that promoters combining elements from more than one promoter may be useful. For example, U.S. Pat. No. 5,491,288 discloses combining a Cauliflower Mosaic Virus promoter with a histone promoter. Thus, the elements from the promoters disclosed herein may be combined with elements from other promoters. Promoters which are useful for plant transgene expression include those that are inducible, viral, synthetic, constitutive (Odell Nature 313: 810-812 (1985)), temporally regulated, spatially regulated, tissue specific, and spatial temporally regulated. Using the regulatory elements described herein, numerous agronomic genes can be expressed in transformed plants. More particularly, plants can be genetically engineered to express various phenotypes of agronomic interest.

### DETAILED DESCRIPTION

One embodiment of the invention provides a method of editing plant genomic DNA, comprising: (i) obtaining a first plant, wherein the first plant is a haploid inducer line of the plant, and wherein said first plant is capable of expressing a DNA modification enzyme and optionally a guide nucleic acid; (ii) obtaining a second plant, wherein the second plant comprises the plant genomic DNA which is to be edited; (iii) pollinating the second plant with pollen from the first plant; and (iv) selecting at least one haploid progeny produced by the pollination of step (c) wherein the haploid progeny comprises the genome of the second plant but not the first plant, and the genome of the haploid progeny has been modified by the DNA modification enzyme and optional guide nucleic acid delivered by the first plant.

In one aspect of the method, the DNA modification enzyme is a site-directed nuclease selected from the group consisting of meganucleases (MNs), zinc-finger nucleases (ZFNs), transcription-activator like effector nucleases (TALENs), Cas9 nuclease, Cfp1 nuclease, dCas9-FokI, dCpf1-FokI, chimeric Cas9-cytidine deaminase, chimeric Cas9-adenine deaminase, chimeric FEN1-FokI, and Mega-TALs, a nickase Cas9 (nCas9), chimeric dCas9 non-FokI nuclease and dCpf1 non-FokI nuclease; and further wherein the guide nucleic acid is a guide RNA.

In another aspect of the method, the edited haploid progeny is treated with a chromosome doubling agent, thereby creating an edited doubled haploid progeny. For example, the chromosome doubling agent is colchicine, pronamide, dithipyr, trifluralin, or another known anti-microtubule agent.

In another aspect of the method, the first plant is a monocot or a dicot. For example, the first plant is a monocot selected from the group consisting of maize, wheat, rice, barley, oats, triticale, sorghum, pearl millet, teosinte, bamboo, sugar cane, asparagus, onion, and garlic. In another aspect, the second plant is a monocot or a dicot. For example the second plant is a monocot selected from the group consisting of maize, wheat, rice, barley, oats, triticale, sorghum, pearl millet, teosinte, bamboo, sugar cane, asparagus, onion, and garlic.

In another aspect of the method, the optional guide RNA is an 18-21 nucleotide sequence and is homologous to a sequence selected from the group consisting of SEQ ID NOs: 2, 4, 8, 21, 23, 25, 29, 32, and 33. In another aspect, the first plant expresses a marker gene. For example, the marker gene is selected from the group consisting of GUS, PMI, PAT, GFP, RFP, CFP, B1, C1, R-nj, anthocyanin pigments, and any other marker gene.

In another aspect of the method, the first plant is a maize plant selected and/or derived from the lines Stock 6, RWK, RWS, UH400, AX5707RS, NP2222-matl, or any of the several other known HI lines.

In one embodiment, the first plant and the second plant are different species. In one aspect, first plant is a wheat plant and the second plant is a maize plant. In another aspect, the first plant is a maize plant and the second plant is a wheat plant.

One object of the invention is a gene-edited plant produced by the method provided.

In another embodiment, the invention provides a method of editing plant genomic DNA, comprising: (i) obtaining a first plant, wherein said first plant is capable of expressing a DNA modification enzyme and optionally a guide nucleic acid; (ii) obtaining a second plant, wherein the second plant comprises the plant genomic DNA which is to be edited; (iii) pollinating the second plant with pollen from the first plant; (iv) applying a composition comprising a lipid or a phospholipase inhibitor immediately preceding, during, or following the pollination of step (iii); and (v) selecting at least one haploid progeny produced by the pollination of step (iii) wherein the haploid progeny comprises the genome of the second plant but not the first plant, and the genome of the haploid progeny has been modified by the DNA modification enzyme and optional guide nucleic acid delivered by the first plant. In one aspect, the composition comprises methyl alpha-linolenoyl fluorophosphonate (MALFP), linoleic acid ethyl ester (LLAEE), linoleic acid (LLA), corn oil, distearoyl-phosphatidyl choline (DSPC), methyl arachidonyl fluorophosphonate (MAFP), Palmityl trifluoromethylketone (PACOCF3), Arachidonyl trifluoromethylketone (AACOCF3), Manoalide, Linolenic acid ethyl ester (LNAEE), Linolenic acid ethyl ester (LNAEE), Oleic acid methyl ester (OAME), Oleic acid ethyl ester (OAEE), Palmitic acid ethyl ester (PAEE), Palmitoleic acid ethyl ester (PLAEE), Linseed oil, corn oil, alpha-Linolenic acid (aLNA), gamma-Linolenic acid (gLNA), Oleic acid, Arachidonic acid, Stearic Acid, 9(Z)-11(E)-conjugated Linoleic acid, or 2-oleoyl-1-palmitoyl-sn-glycero-3-phospho-ethanolamine.

In another embodiment, the invention provides a method of editing plant genomic DNA, comprising: (i) obtaining a first plant, wherein said first plant is capable of expressing a DNA modification enzyme and optionally a guide nucleic acid; (ii) obtaining a second plant, wherein the second plant comprises the plant genomic DNA which is to be edited; (iii) crossing the first plant with the second plant; and (iv) selecting at least one haploid progeny produced by the crossing of step (iii) wherein the haploid progeny comprises the genome of the second plant but not the first plant, and the genome of the haploid progeny has been modified by the DNA modification enzyme and optional guide nucleic acid delivered by the first plant. In one aspect, the first plant acts as the female parent in the cross of step (iii). In another aspect, the first plant comprises a mutation in a CENH3 gene, an ig1 gene, or another mutation conferring paternal-haploid inducing systems.

### Examples

### I. Producing New Haploid Inducer Lines Comprising the Editing Machinery.

We transformed a transformable line of maize called NP2222 with a TALEN construct, and separately transformed this line with a Cas9 and guide RNA construct. The TALEN construct (pBSC22808 (SEQ ID NO: 5), with TALENs targeting cleavage within target sequence, 5′-TCCAGGGTCAACGTGGAGACAGGGAGGTACGAACCGGTGACTGGCGAAGGAAG CA-3′, SEQ ID NO: 6; TALEN recognition sequence underlined) and the Cas9 construct (pBSC23123 (SEQ ID NO: 7) with guide RNA sequence of xZmPLAIIA, 5′-GGGTCAACGTGGAGACAGGG-3′, SEQ ID NO: 8) were designed to target mutations into the fourth exon of maize gene called MATRILINEAL (MATL; GRAMENE ID: GRMZM2G471240). This gene, when mutated at the target site by the TALEN or by the Cas9 and guide RNA, is knocked out, resulting in a loss of function of the protein product. We previously established that lines that are homozygous for loss of function mutations in MATL are haploid inducer lines, meaning that when they are used as pollen donors in crosses, they induce the formation of haploids on the resulting ears (see P.C.T. Patent Application No. PCT/US2016/62548, filed Nov. 17, 2016, incorporated herein by reference in its entirety).

We produced several events and self-pollinated them to make T1 seed. We grew up T1 individuals from event MZET152408A042A. We recovered five T1 progeny that retained two copies of the Cas9 and guide RNA editing machinery stably transformed, and were also homozygous mutant for the MATL gene. See Table 1.

TABLE 1
New HI lines comprising the genome editing machinery.

| New HI Line | wt MATL | Cas9 | Mutation |
|---|---|---|---|
| Individual ID | Presence | Presence | in MATL |
| USR01283349 | − | + | 13 bp deletion, homozygous |
| USR01283378 | − | + | 13 bp deletion, homozygous |
| USR01283388 | − | + | 8 bp deletion, homozygous |
| USR01283391 | − | + | 8 bp deletion, homozygous |
| USR01283398 | − | + | 13 bp deletion, homozygous |

The MATL mutations are detected using a TaqMan assay, which amplifies the wildtype copy of MATL (referred to herein as MATL or wt-MATL; these terms are used interchangeably throughout). When both copies of MATL are mutated, this assays reads negative (i.e., “−”). The Cas9 and guide RNA editing machinery were stably inserted via Construct 23123 (SEQ ID NO: 7). We sequenced the mutations in MATL via PCR and subcloning. Four colonies of each PCR product was sequenced, and all of the colonies for a given individual had the same sequence, indicating these plants are all homozygous mutant for the MATL allele (also referred to herein as matl when referencing the 4 basepair insertion in MATRILINEAL found in Stock6 and other Stock6-derived lines, or μMATL when referencing any other human-induced mutation in MATRILINEAL). There were two plants that had 8 bp deletions, and three plants that had 13 bp deletions.

### II. Using the New HI Lines as Male Parents and Progeny Analysis.

We crossed the above new HI plants as male pollen donors to a female tester line, which contained a recessive color marker but were wild type for the MATL gene. The male haploid inducer line is homozygous wild type for the same color marker. This female line was thus a non-haploid inducer and were homozygous wild-type for the MATL gene but homozygous mutant for the color marker. We recovered seeds from the crosses, and germinated seedlings therefrom.

Progeny seedlings were subjected to several assays. Progeny seedlings were scored as diploids if they do not exhibit the color marker (because the recessive marker is complemented by the male inducer DNA). Progeny seedlings were scored as putative haploids if they do exhibit the color marker because the recessive marker is not complemented. Of the 2656 seeds planted, we used the color assay and identified 90 seedlings as putative haploids.

We further analyzed the 90 putative haploids for presence of the wildtype MATL gene using a Taqman marker assay. Of these, 82 were positive for MATL, meaning they were not edited by the editing machinery provided by the male parent. The remaining 8 putative haploid seedlings were negative for wildtype MATL using the Taqman marker, indicating that they may have been edited by the editing machinery provided by the male parent.

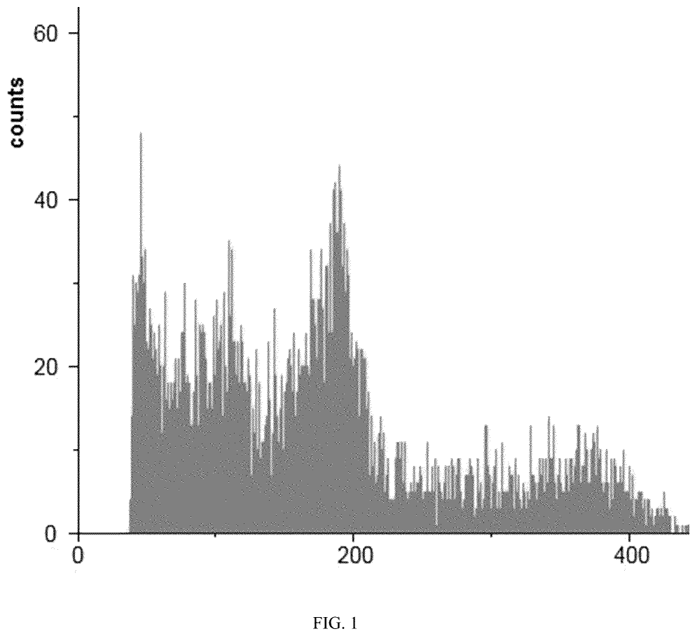

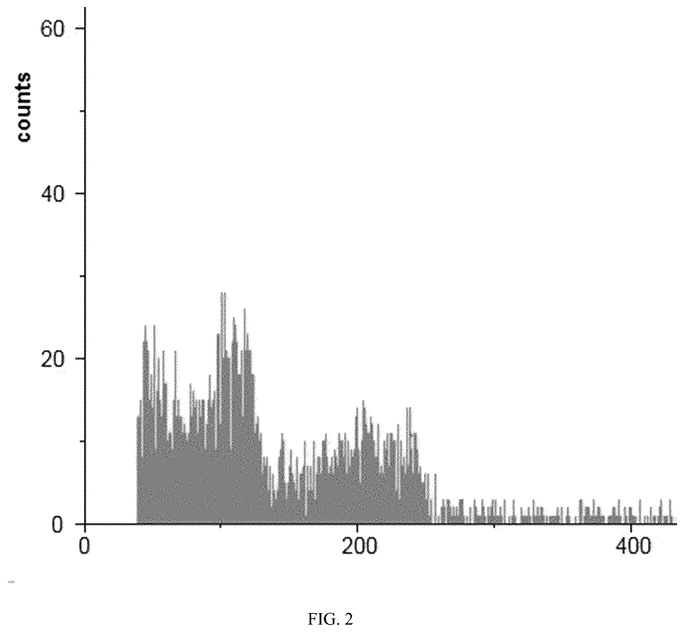

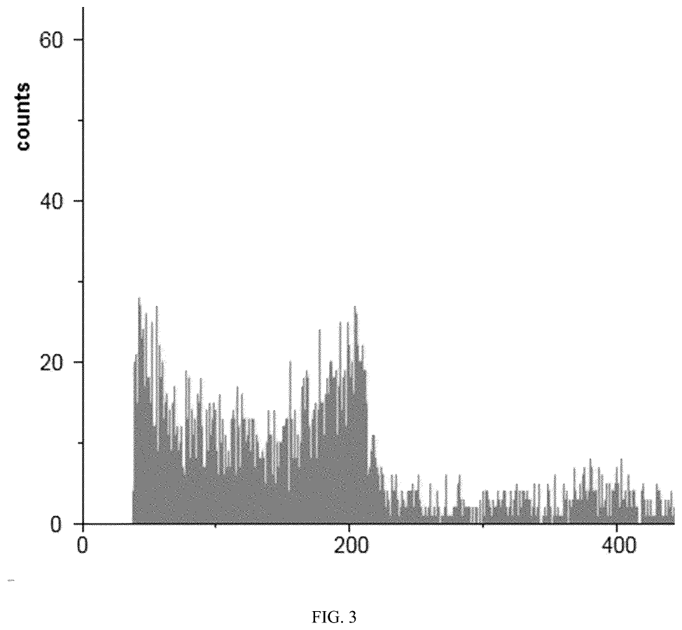

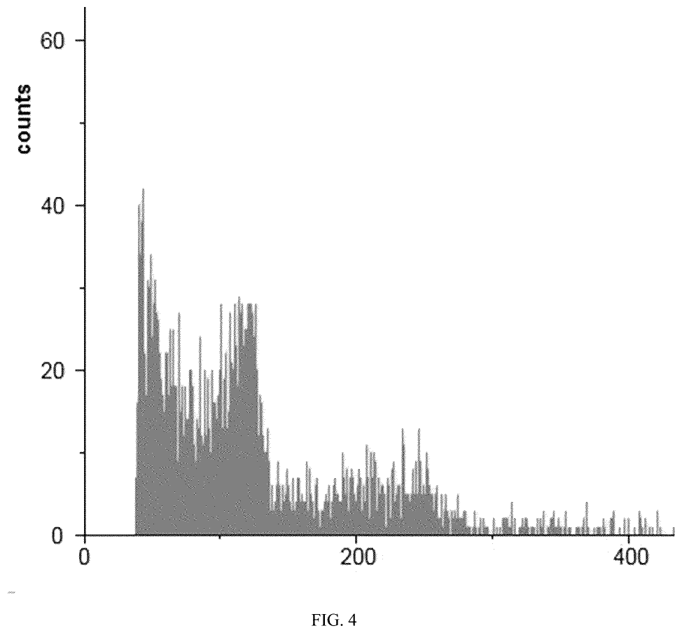

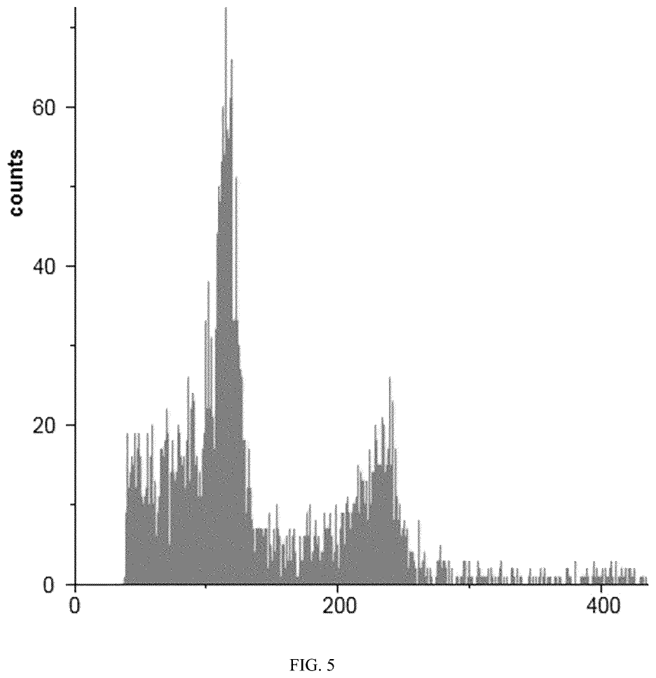

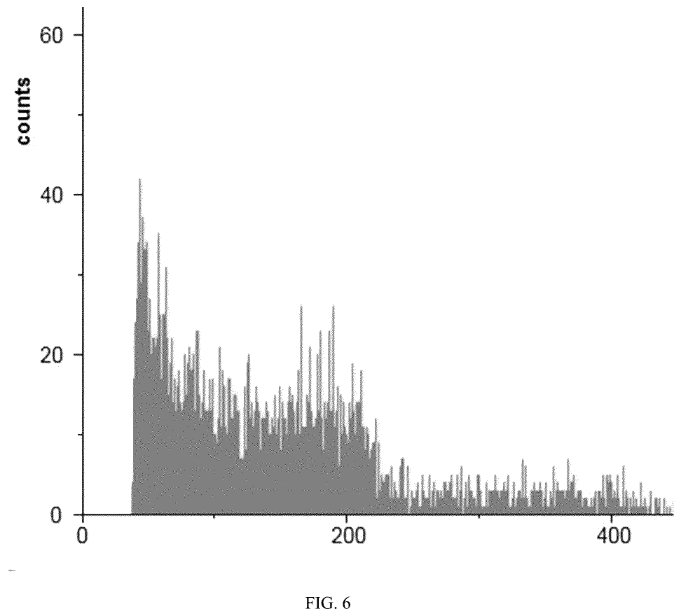

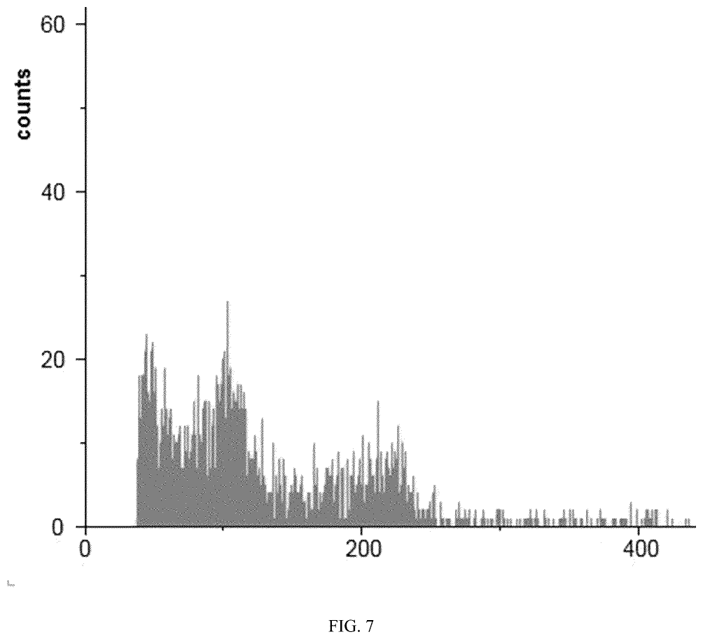

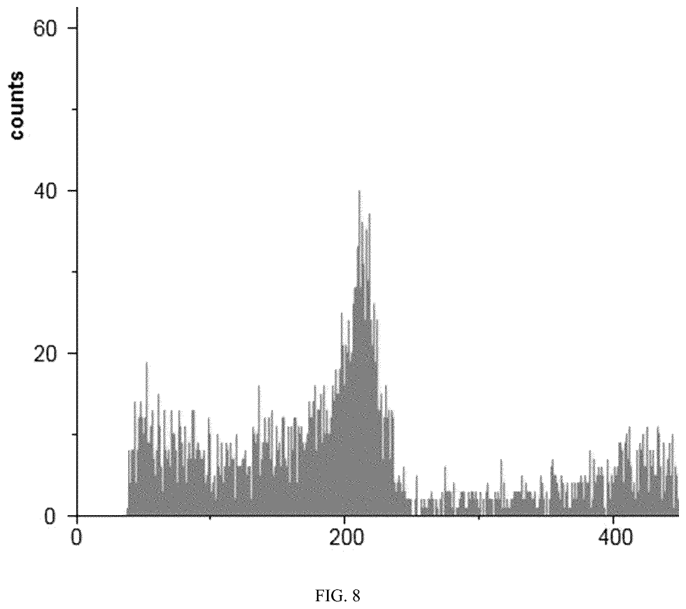

We performed ploidy analysis via Flow Cytometry on these 8 putative, edited haploid seedlings using leaf tissue in a ploidy analyzer. See FIGS. 1-8. We found that four of them were true haploids, while the others were actually diploids. As we discuss below, we ran PCR and sequenced the mutations in the MATL gene in these four true haploids as well as for plant USR01350337-2 which, according to the MATL Taqman assay, was not edited by the genome editing machinery.

The finding that there were four diploids among the 90 putative haploids was not unexpected—the seedling assay is not perfect and there are occasional false positives. We tested the 90 haploids for the presence of the Cas9 construct (Construct 23123), and found it was missing in 86 out of 90, including the four true haploids above. In contrast, the four edited diploids that we found during the ploidy analysis all had the Cas9 construct present, confirming their status as hybrid diploids that were falsely identified by the haploid seedling assay as being haploids.

We then used the leaf tissue to isolate genomic DNA and ran a PCR reaction to sequence the MATL gene in those four true haploid, putative edited individuals, specifically focusing on the sequence flanking the guide RNA target mutagenesis site. This was to determine the nature of the edits that may or may not have occurred there. We sub-cloned the PCR fragment using commercially-available TOPO Blunt IV kit, and sequenced at least four colonies each (forward and reverse sequencing). See Table 2, below, for comparisons of the edited alleles and the reference wt-MATL allele.

TABLE 2
Comparing the Edited Alleles against wt-MATL.

| | | Sequence (corresponds to | SEQ ID |
|---|---|---|---|
| Individual ID | Allele Type | 1126-1166 of SEQ ID NO: 19) | NO: |
| NP2222 | wt-MATL | AGGGTCAACGTGGAGACAGGGAGGTACGA | 9 |
| Stock6 | matl | ACCGGTGACTGGAGGGTCAACGTGGAGAC | 10 |
| Allele 1 | | AGGCGAGGAGGTACGAACCGGTGACTGG | |
| USR01350333-3 | edited | AGGGTCAACGTGGAGACAAGGGAGGTACG | 11 |
| Allele 1 | | AACCGGTGACTGG | |
| USR01350333-3 | PCR | AGGGTCAACGTGGA:::::::::::::GA | 12 |
| Allele 2 | contamination | ACCGGTGACTGG | |
| USR01350344-2 | edited | AGGGTCAACGTGGAGAC:GGGAGGTACGA | 13 |
| Allele 1 | | ACCGGTGACTGG | |
| USR01350344-2 | PCR | AGGGTCAACGTGGA:::::::::::::GA | 14 |
| Allele 2 | contamination | ACCGGTGACTGG | |
| USR01350343-1 | edited | AGGGTCAACGTGGAGACAAGGGAGGTACG | 15 |
| Allele 1 | | AACCGGTGACTGG | |
| USR01350328-1 | edited | AGGGTCAACGTGGAGAC:GGGAGGTACGA | 16 |
| Allele 1 | | ACCGGTGACTGG | |
| USR01350337-2 | not edited | AGGGTCAACGTGGAGACAGGGAGGTACGA | 17 |
| Allele 1 | | ACCGGTGACTGG | |
| USR01350337-2 | PCR | AGGGTCAACGTGGA:::::::::::::GA | 18 |
| Allele 2 | contamination | ACCGGTGACTGG | |

Individual USR01350333-3 produced an edited MATL allele with an insertion of alanine at basepair 1143 of the cDNA sequence (underlined in Table 2). This would be sufficient to cause a frameshift in the coding sequence, which would produce a premature STOP codon. What we previously thought was Edited Allele #2 of USR01350333-3 (a 13 basepair deletion of GACAAGGGAGGTAC) was actually the result of PCR contamination. After resequencing, we confirmed that this plant only has one edited allele, and it was found in 6 out of 6 colonies.

This alleles is novel in that it is not in either the male or the female parent plant of this individual. The male parent ID for this individual was USR01283391, and that plant was found to be homozygous for an 8 bp deletion.

Individual USR01350344-2 provides a deletion of A (a deletion of basepair 1143 of the wild type cDNA sequence). This mutation would be sufficient to cause a frame-shift in the coding sequence, and produce a premature STOP codon. After resequencing and discovering the PCR contamination, we confirmed this was found in 6 out of 6 colonies. Previously identified as Edited Allele #2 of USR01350344-2, this was identified as PCR contamination.

Individual USR01350343-1 provides an insertion of A at basepair 1143 of the cDNA sequence. This would be sufficient to cause a frame-shift in the coding sequence, and produce a premature STOP codon. This was found in 4 out of 4 colonies.

Individual USR01350328-1 provides a deletion of A (a deletion of basepair 1143 from the wild type cDNA sequence). This mutation would be sufficient to cause a frame-shift in the coding sequence, and produce a premature STOP codon. It was found in 4 out of 4 colonies.

Individual USR01350337-2 had no change: its sequence was 100% identical to that of wt-MATL.

In summary, we found that 4 out of 86 confirmed haploids had mutations in the MATL gene. We have confirmed that these plants are haploids and do not contain any Cas9 DNA. It is clear that the Cas9 transgene has been eliminated along with the rest of the male-derived DNA during embryogenesis, and that edits have occurred to the female (egg cell-derived) genome in the process of embryogenesis.

We know that the edits are novel and occurred in the female genome in the process of embryogenesis because the haploid inducer line typically makes maternal haploids and we have confirmed that these are indeed haploids. One might try to argue that there is a chance that these are actually paternal haploids, and that the edits we are seeing are actually edits that were already present in the paternal DNA. However, we can prove that this is not the case. First, the mutations do not match those of the paternal parent. This can clearly be seen in Table 3 and 4 (shown below). The edited haploid plant USR01350343-1 was homozygous for an insertion of a single nucleotide (an “A”), but the male parent plant had a deletion of 13 nucleotides. Similarly, plant USR01350328-1 was homozygous for a deletion of an A, but the male parent had a deletion of 13 nucleotides. These examples, taken together, prove that during the haploid induction process, it is possible to have editing of the maternal genome occur, resulting in the formation of edited maternal haploids. According to these and based on the assay detecting MATL presence and the confirmation via ploidy analysis, and using the Cas9 transgene on the male side under control of the maize ubiquitin promoter, the rate of editing during the haploid induction process is about 4/86, or 4.65%.

Furthermore, the rate of editing during haploid induction may be very different when using different haploid inducer lines or using wide crosses. It appears that both haploid induction in maize using MATL mutant lines and wide crosses in barley, wheat, or other crops all work via similar mechanisms: fertilization is followed by genome elimination. It also appears that the time period between fertilization and genome elimination is long enough for the editing machinery to edit the target gene in the genome of the line to which the inducer line has been hybridized (the target germplasm). It is noted that the choice of promoter driving expression of the stably transformed editing proteins system may have a large impact on the rate of editing in haploids. We used a constitutive sugarcane promoter (prSoUbi4) but other promoters driving high or specific expression in the embryo sac, the egg cell, in the pollen, or in sperm cells might be more effective, particularly in the case of wide crosses, in which the male DNA is eliminated in a much more robust and rapid fashion than in intraspecific haploid inducer systems like the maize haploid inducer system or CENH3 type haploid inducer systems. In other words, during a wide cross, for instance when crossing maize pollen on to wheat ears, which is done in order to induce wheat maternal haploids, it might work best to have the editing machinery in the maize pollen driven by a promoter that has strong pollen or sperm cell expression, perhaps in addition to zygote expression, so that abundant editing machinery (RNA and protein) is delivered and present in the zygote cell and during the subsequent two, four, or eight cell embryo stage, even if the male DNA is eliminated or lost very quickly.

TABLE 3
Haploid Progeny Produced

| Individual Progeny | wt MATL | Ploidy | Cas9 | |
|---|---|---|---|---|
| ID code | Presence | Analysis | Presence | Allele 1 |
| USR01350333-3 | − | Haploid | − | insertion of A |
| USR01350344-2 | − | Haploid | − | deletion of A |
| USR01350343-1 | − | Haploid | − | insertion of an A |
| USR01350328-1 | − | Haploid | − | deletion of A |
| USR01350337-2 | + | Haploid | − | no mutation |
| USR01350334-3 | − | Diploid | + | |
| USR01350333-10 | − | Diploid | + | |
| USR01350341-1 | − | Diploid | + | |
| USR01350321-3 | − | Diploid | + | |

TABLE 4
Male Parent Information and Their Progeny

| | wt MATL | Sequencing (# | Cas9 | |
|---|---|---|---|---|
| Male Parent ID | Presence | colonies) | Presence | Progeny ID |
| USR01283391 | − | deletion of 8 nt (4) | + | USR01350333-3 and USR01350333-10 |
| USR01283349 | − | deletion of 13 nt (4) | + | USR01350344-2, USR01350328-1 and |
| | | | | USR01350321-3 |
| USR01283378 | − | deletion of 13 nt (4) | + | USR01350343-1 and USR01350341-1 |
| USR01283398 | − | deletion of 13 nt (4) | + | USR01350337-2 |
| USR01283388 | − | deletion of 8 nt (4) | + | USR01350334-3 |

### III. Simultaneous Haploid Induction and Editing in Elite Maize Inbred Lines.

A transformable haploid inducer line, NP2222-HI, RWK, RWS, or UH400 or Stock6 or any other haploid inducer line, all of which already have the mutant versions of MATL, is stably transformed with construct expressing genome modification system such as Cas9+ guide RNA (Cong, L. et al. 2013. Multiplex genome engineering using CRISPR/Cas systems. Science 339, 819-823), dCas9-FokI+ guide RNA (Tsai, S. Q. et al. 2014, Dimeric CRISPR RNA-guided FokI nucleases for highly specific genome editing. Nature Biotechnol. 32, 569-576), TALEN (Li et al., 2012, High-efficiency TALEN-based gene editing produces disease-resistant rice. Nature Biotech. 30, 390-392), engineered meganuclease (Gao et al., 2010, Heritable targeted mutagenesis in maize using a designed endonuclease. Plant Journal. 61:176-187), zinc finger nuclease (Shukla et al. 2009. Precise genome modification in the crop species Zea mays using zinc-finger nucleases. Nature 459, 437-441), dCas9-cytidine deaminase (Komor et al. 2016, Programmable editing of a target base in genomic DNA without double-stranded DNA cleavage. Nature doi:10.1038/nature17946) or any other genome modification system. The transgenic haploid inducer line also expressing the editing machinery is then used as pollen donor to create mutations and haploids in target lines via outcrossing. Haploid embryos or seeds are then recovered, identified as haploids, and tested for the edits at the target site (whatever target site is chosen by virtue of the TALEN construct design or the Cas9 guide RNA design). Haploids containing the desired edits is chromosomally doubled using standard procedures using standard means such as colchicine, trifluralin or other chromosome doubling agent. Identification of the induced haploids can be simplified by using a color marker as is typically done in corn doubled haploid production—this color marker can display in the resulting embryos, seeds, seedlings, or adult plant. Presence of mutations at the target site can be checked by sequence analysis (DNA sequencing), by marker analysis, or by phenotype. Because there is only one copy of the DNA to mutate in haploid plants, recessive phenotypes should display so that could be another way to identify the haploids that were edited.

A. Mutagenesis of VLHP Targets in Elite Maize Inbred Line with Transgenic Editing Locus Generated Directly in a Haploid Inducer Line.

VLHP1 and VLHP2 are homeodomain-leucine zipper I-class homeobox genes and members of a class of proteins that is unique to plants. The HD domain is involved in DNA binding whereas the Zip domain is involved in protein homo- and hetero-dimerization. HD-Zip I proteins are generally involved in responses related to abiotic stress, abscisic acid (ABA), blue light, de-etiolation and embryogenesis (Elhiti and Stasolla, 2009. Structure and function of homodomain-leucine zipper (HD-Zip) proteins. Plant Signal Behav. 4: 86-88). VLHP1 and VLHP2 are in the same gene family as Grassy Tillers1 (GT1). GT1 promotes lateral bud dormancy and suppresses elongation of lateral ear branches in maize.

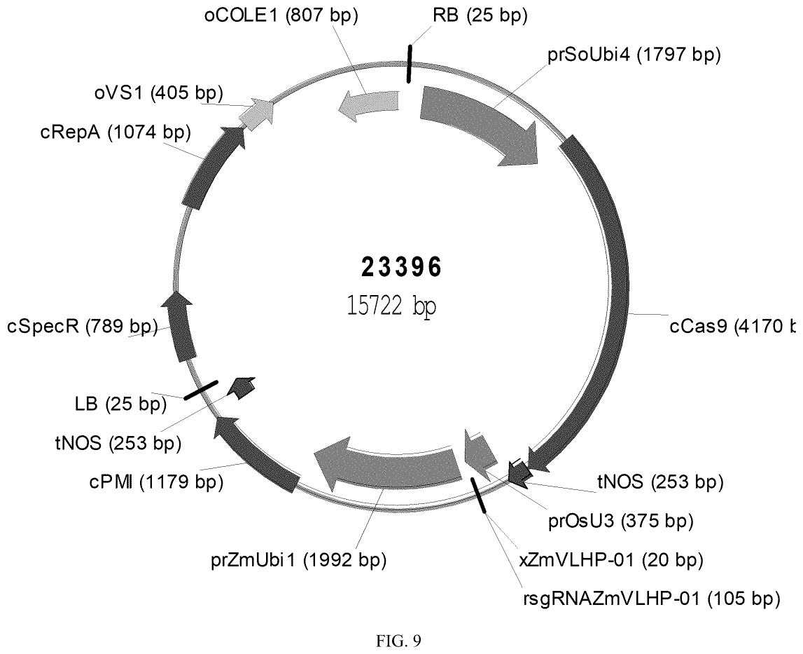

In this example, vector 23396 (SEQ ID NO: 1; see also FIG. 9) for expressing Cas9 and single guide RNA (sgRNA) was made to target maize VLHP1 (GRMZM2G104204) and its homolog VLHP2 (GRMZM2G062244) genes. Vector 23396 expresses a sgRNA with 20-nucleotide targeting sequence xZmVLHP-01 (5′-GCAGGAGGCGTCGAGCAGCG-3′, SEQ ID NO: 2). xZmVLHP-01 targets both VLHP1 and VLHP2 genes at the second exon. Vector 23396 was introduced into a transformable haploid inducer line NP2222-HI using Agrobacterium-mediated transformation with mannose selection. NP2222-HI was derived from crossing of transformable maize inbred line NP2222 with Stock 6 derivative line RWKS to introgress the haploid induction (HI) locus. NP2222-HI has an average haploid induction rate of about 9.2%.

NP2222-HI transformants from vector 23396 were assayed for modification of genomic VLHP target sequences (5′-GCAGGAGGCGTCGAGCA/GCG-3′; SEQ ID NO: 2). The slash (“/”) represents the Cas9 cleavage position. Target locus editing activity was determined using quantitative PCR Taqman method as described before (WO2016106121, incorporated herein by reference). Transgenic lines with high target site modification activities—i.e., both VLHP1 and VLHP2 genes were modified, and preferably containing single copy transgene—were selected for further studies and used for crossing or progeny production.

Pollen from T0 transformants of 23396 is used directly to pollinate ears of elite inbred line ID5829 or other maize lines including sweet corn lines to induce production of haploid embryos. Alternatively, T0 transformants of 23396 in NP2222-HI background are selfed to produce progeny lines carrying homozygous transgene and pollen from the progeny plants are used to pollinate other corn lines to induce haploid embryo formation. The induced haploid embryos are extracted from kernels and placed on embryo rescue media for direct germination or allowed to mature to form seeds. Tissues from the induced haploid embryos and the resulting plants are assayed to determine if editing has occurred in the VLHP target sequences. If the induced haploid embryos or plants contain desired mutations, chromosome doubling treatment is applied to produce doubled haploid lines from them. For example, using embryo rescue method, embryos are extracted from elite line ID5829 ears pollinated with transgenic haploid inducer line carrying 23396 editing locus at 18-22 days after pollination (“DAP,” extraction between 10-25 DAP is theoretically possible). DNA is isolated from germinated haploid seedlings and used for assay. Colchicine treatment is applied to seedling for chromosome doubling. Alternatively, chromosome doubling agent can be applied to the isolated embryos during germination. DNA is extracted from germinated seedlings and used for determining that mutation has occurred at the xZmVLHP-01 target sequence.

Alternate methods are available. One could allow the seed to mature and select haploids later by another phenotype. One could let the seed dry down and at a later date germinate the seeds to determine haploids without a marker (e.g., using plant size rather than a gene conferring a color marker), at which point one would test for edits and apply chromosome doubling agents where appropriate. This method may have its advantages in that embryo screening and/or rescue is avoided.

B. Mutagenesis of GW2 Targets in Elite Maize Inbred Line with Transgenic Editing Locus Introduced Directly in a Haploid Inducer Line.

A mutation in DA2, an E3-ubiquitin ligase gene, in rice resulted in larger seeds (Song et al., 2007). Rice DA2 has 2 maize homologs, GW2-1 (GRMZM2G170088) and GW2-2 (GRMZM2G007288). The maize genes are 94% identical at the protein level and 90% identical at the DNA level. GRMZM2G170088 has a large 177 bp insert (59 aa) in comparison with GRMZM2G007288.

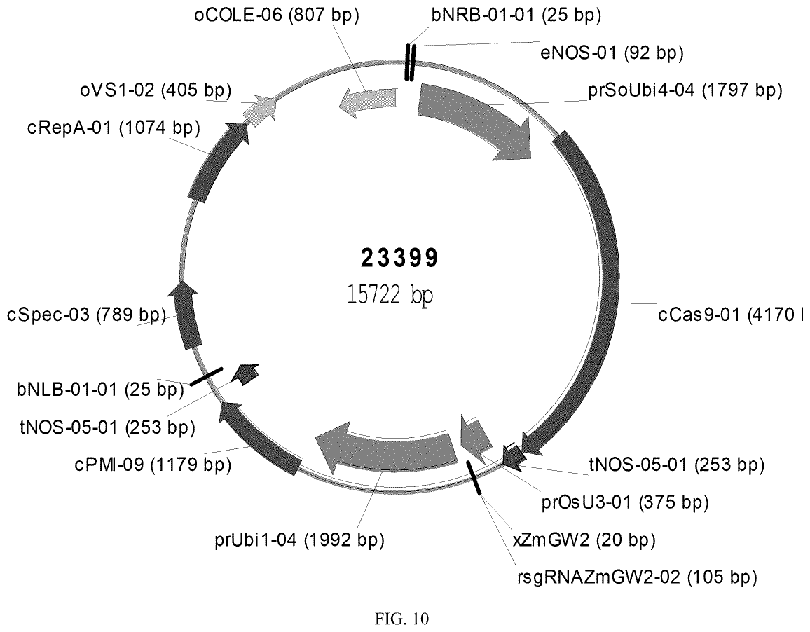

In this example, vector 23399 (SEQ ID NO: 3, see also FIG. 10) was made for expression of Cas9 and sgRNA to target both maize GW2-1 (GRMZM2G170088) and its homolog GW2-2 (GRMZM2G007288) genes. Both GW2-1 and GW2-2 genes contain target sequence xZmGW2-02 (5′-AAGCTCGCGCCCTGCTACCC-3′, SEQ ID NO: 4) in exon 1 and this sequence was used to design sgRNA expressed from vector 23399. Binary vector 23399 expresses single guide RNA (sgRNA) with 20-nucleotide targeting sequence xZmGW2-02 fused to single guide RNA scaffold comprising of both crRNA and tracrRNA. Vector 23399 was introduced into a transformable haploid inducer line NP2222-HI using Agrobacterium-mediated transformation with mannose selection. NP2222-HI was derived from crossing of transformable maize inbred line NP2222 with Stock 6 derivative line RWKS to introgress the haploid induction (HI) locus.

NP2222-HI transformants of vector 23399 were assayed for modification of genomic GW2-2 target sequences (5′-AAGCTCGCGCCCTGCTA/CCC-3′, SEQ ID NO: 4; the slash (“/”) indicates the Cas9 cleavage position). Target sequence editing activity was determined using quantitative PCR Taqman method as described before (WO2016106121). Transgenic lines with high target site modification activities—i.e. both GW2-1 and GW2-2 genes were modified, and preferably containing single copy transgene—were selected for further studies and used for crossing or progeny production.

Pollen from T0 transformants of 23399 is used directly to pollinate ears of elite inbred line ID5829 or other maize line including sweet corn lines to induce production of haploid embryos. Alternatively, T0 transformants of 23399 in NP2222-HI background are selfed to produce progeny lines carrying homozygous transgene and pollen from the progeny plants are used to pollinate other corn lines to induce haploid embryo formation. The induced haploid embryos are extracted from kernels and placed on embryo rescue media for direct germination or allowed to mature to form seeds. Tissues from the induced haploid embryos and the resulting plants are assayed to determine if editing has occurred in the maize GW2 target sequences. If the induced haploid embryos or plants contain desired mutations, chromosome doubling treatment is applied to produce doubled haploid lines from them. For example, using embryo rescue method, embryos are extracted from elite line ID5829 ears pollinated with transgenic haploid inducer line carrying 23396 editing locus at 18-22 days after pollination. DNA is isolated from germinated haploid seedlings and used for assay. Colchicine treatment is applied to seedling for chromosome doubling. Alternatively, chromosome doubling agent can be applied to the isolated embryos during germination. DNA is extracted from germinated seedlings and used for determining if mutation has occurred at the xZmGW2-02 target sequence. Alternately, one could allow the seed to mature and select haploids later by another phenotype. One could even let the seed dry down and at a later date germinate the seeds to determine haploids without a marker (e.g., using plant size rather than a gene conferring a color marker), at which point one would test for edits and apply chromosome doubling agents where appropriate. This method may have its advantages in that embryo screening and/or rescue is avoided.

### IV. Simultaneous Haploid Induction and Editing in Corn, Rice, Sunflower, or any Other Crop Via Chemical-Based Haploid Induction

Any line of corn, rice, wheat, tomato, sunflower, barley, or any other crop is transformable with the editing construct (Cas9 plus guide RNAs designed to mutate a particular target site) and then optionally make the editing construct either heterozygous or homozygous (via self-pollination of the transformed event), and then using lipid or oil applications during outcrossing (pollination onto target lines) in order to induce de novo haploids and simultaneously edit the target sites in the target genomes. These lipid applications have the ability to induce haploids when applied to pollen, silks, flowers, or tassels of any plant—regardless of male parent. In particular, the male parent is not required to have any mutations in the MATL gene (i.e., it can be homozygous wild type for the MATRILINEAL gene). These lipid applications induce haploids de novo, without any genetic requirement on behalf of either parent. See P.C.T. Patent Application No. PCT/US2016/62548, incorporated herein by reference in its entirety. The mechanism of de novo haploid induction via lipid spray apparently works the same way as it does in matl mutant (genetic haploid inducer) lines: via chromosome elimination post-fertilization. Haploid progeny are isolated and checked for the induced mutations (caused via the editing process) and then doubled to make edited, doubled haploid plants.

V. Mutagenesis of Target Sequences in Elite Field Corn and Sweet Corn Inbred Lines with Transgenic Editing Locus Introgressed into a Haploid Inducer Line.

Transgenic locus expressing genome editing machinery can also be generated in conventional transformable maize line without haploid inducing activity such as A188, Hi-II or NP2222 and then introgressed into haploid inducer line such as NP2222-HI, RWK, RWKS, RWS, or UH400 or Stock6 or any other haploid inducer line.

In this example, maize inbred line NP2222 is transformed with VLHP Cas9-sgRNA vectors (23396 and 23397) and GW2 Cas9-sgRNA vectors (23398 and 23399). Vectors 23396 and 23399 have been described in previous examples (Example IIIA and Example IIIB). Vector 23397 (SEQ ID NO: 20) is identical to 23396 except the gRNA-coding sequence xZmVLHP-01 (5′-GCAGGAGGCGTCGAGCAGCG-3′, SEQ ID NO: 2) is replaced with xZmVLHP-02 (5′-GCTGGAGCTGAGCTTCCGGG-3′, SEQ ID NO: 21). Vector 23398 (SEQ ID NO: 23) is identical to 23399 except the gRNA-coding sequence xZmGW2-02 (5′-AAGCTCGCGCCCTGCTACCC-3′, SEQ ID NO: 4) in 23399 is replaced by xZmGW2-01 (5′-GAGCGGTTCACGCGGCCGCA-3′, SEQ ID NO: 23). These vectors were introduced into Agrobacterium strain LBA4404 (pVGW7). The resulting Agrobacterium strain containing vector 23396, 23397, 23398, or 23399 was used to transform immature embryos of transformable elite inbred line NP2222. Calli were induced from infected immature embryos and selected on mannose media to recover transgenic calli. Transgenic calli were placed on regeneration and rooting media to recover transgenic plants expressing the CRISPR-Cas9 editing machinery. Transgenic plants were assayed for transgene copy number and moved to greenhouse for seed production.

Single copy transformants of vector 23396 (MZET154902A004A, MZET154902B006A), 23397 (MZET154903B009A, MZET154903B012A), 23398 (MZET154904B005A, MZET154904B014A) and 23399 (MZET154905A002A, MZET154905A010A) were identified and backcrossed with non-transgenic NP2222. Ears of transgenic progeny plants containing T-DNA insert of each of the above vectors were pollinated with pollen of haploid inducer line RWKS to produce F1 progeny. F1 progeny containing transgenic locus and haploid induction locus were identified by genotyping assays and self-pollinated to produce F2 progeny seeds. F2 progeny seeds were planted and seedling plants assayed to identify plants homozygous for transgenic Cas9-sgRNA locus (assay #2540) and haploid induction locus (assay #2827) with qPCR Taqman assays.

Lines homozygous for the haploid induction locus and preferably homozygous transgenic 23396, 23397, 23398, and 23399 Cas9-sgRNA editing locus were used to pollinate ears from target elite field corn line ID5829 and sweet corn lines (SWC726 or SWC412F) for haploid induction. Induced haploid embryos were isolated from pollinated ID5829, SWC412F, SWC726 ears and geminated on embryo rescue media. Alternatively, pollinated ears were allowed to mature and kernels with haploid embryos were germinated. Leaf samples were collected and analyzed with Taqman assay to identify plants containing mutations in VLHP and GW2 genes but absence of genetic components from induction line such as transgenic Cas9-sgRNA or other non-transgenic marker gene sequences. Identified haploid plants with targeted GW2 or VLHP gene mutations were treated with colchicine for chromosome doubling to recover doubled haploid plants for seed production. Alternatively, extracted haploid embryos can be treated with chromosome doubling agent such as colchicine and the resulting plants are analyzed for ploidy level and presence of targeted mutations in GW2 or VLHP genes. Plants with targeted GW2 and VLHP gene mutations are grown to maturity for seed production and further progeny evaluation.

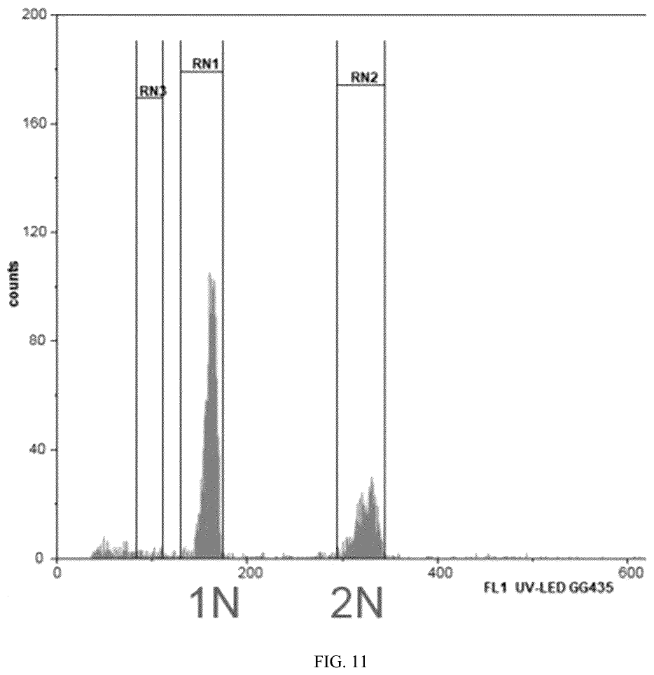

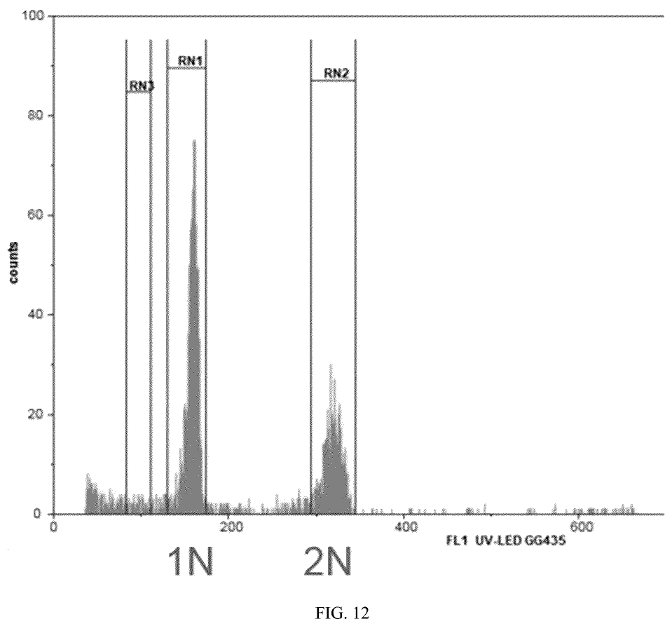

For example, edited haploid lines (JSER82A056 and JSER82A063) were identified from crosses between sweet corn line SWC412F ears pollinated with haploid inducer containing 23399 Cas9-sgRNA transgene. Line JSER82A056 has both GW2-01 and GW2-02 target genes mutated, whereas line JSER82A063 only has GW2-02 gene mutated (See Table 5). Neither of these lines contain Cas9 transgene (assay #2540 for Cas9 or #1750 for PMI selectable marker gene) or haploid inducer gene (assay #2827) as the male genome has been eliminated from the haploids. Ploidy level analysis confirmed that both lines are haploids (FIGS. 11 and 12). Note that wildtype (“WT”) genes in the haploids have a copy number of “2” and mutant will be “0” since the copy call is relative to the endogenous ADH gene copy number. Therefore, haploid lines carrying WT unedited GW2-01 or GW2-02 genes will have a copy call of “2.” WT haploid inducer locus will have copy call of “2” for assay #2826 and “0” for assay #2827 (haploid inducer variant). If a corn plant line is a diploid between sweet corn and transgenic inducer, it will be heterozygous for the haploid inducer gene and thus have copy call of “1” for both assay #2826 and assay #2827.

TABLE 5
Progeny zygosity analysis from crosses. Taqman analysis results showing
the lines do not contain transgene or haploid inducer locus from
pollen donor, but have edits in GW2-01 and/or GW2-02 targets.

| | | Allele: | | | | | |
|---|---|---|---|---|---|---|---|
| | | | | | | | RWK |
| | | | | CRISPR | CRISPR | | (Haploid |
| | | | | target | target | | Inducer) |
| | | | | in GW2- | in GW2- | pPLAIIa | allele |
| | | cCas9- | CPMI- | 01 | 02 | WT | of |
| | | 01 | 09 | (23399) | (23399) | allele | pPLAIIa |
| | | Assay ID: | | | | | |
| | | 2540 | 1750 | 3065 | 3095 | 2826 | 2827 |
| | Construct | Copy# | Copy# | Copy# | Copy# | Copy# | Copy# |
| Plant ID | ID | level | level | level | level | level | level |
| 1-copy control | | + | 1 | ND | ND | 1 | 1 |
| wild type control | | 0 | 0 | 2 | 2 | 2 | 0 |
| JSER82A056 | 23399 | 0 | 0 | 0 | 0 | 2 | 0 |
| JSER82A063 | 23399 | 0 | 0 | 1 or 2 | 0 | 2 | 0 |
| JSER85A021 | 23399 | 0 | 0 | 0 | >2 | 2 | 0 |
| JSER85A022 | 23399 | 0 | 0 | 0 | >2 | 2 | 0 |
| JSER85A024 | 23399 | 0 | 0 | 0 | >2 | 2 | 0 |
| JSER85A027 | 23399 | 0 | 0 | 0 | >2 | 2 | 0 |
| JSER85A037 | 23399 | 0 | 0 | 0 | >2 | 2 | 0 |
| JSER85A039 | 23399 | 0 | 0 | 0 | >2 | 2 | 0 |
| JSER85A044 | 23399 | 0 | 0 | 0 | >2 | 2 | 0 |
| JSER85A055 | 23399 | 0 | 0 | 0 | >2 | 2 | 0 |

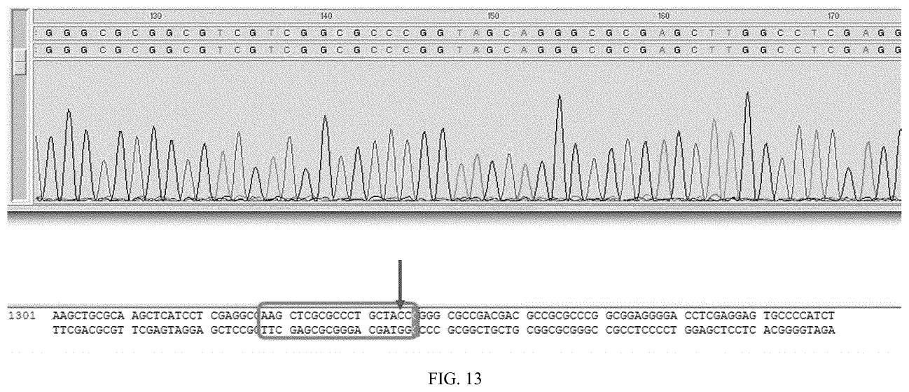

To further confirm target-specific editing in these haploid lines, GW2-02 target region was amplified from JSER82A063 by PCR and the PCR product was sequenced. A single base C was deleted in JSER82A063 in comparison with the WT sequence precisely at the Cas9 cleavage site (FIG. 13). These results clearly demonstrated that editing machinery brought into the egg cell from the male gametophyte can edit the female genome before the male genome is eliminated after double fertilization to form haploid embryo. Candidate edited haploid lines without transgene were treated with injection of 0.125% colchicine in 0.5% DMSO or seedling drenching in 0.06% colchicine solution (Eder and Chalyk, 2002, In vivo haploid induction in maize. Theor. Appl. Genetics 104:703-708). Treated lines were planted in soil and grown in greenhouse for progeny seed production.

### VI. Simultaneous Haploid Induction and Editing in Wheat and Other Monocots Via Wide Cross.

Haploid induction is also achieved using interspecific or intergeneric wide crosses (Kasha and Kao, 1970, High frequency haploid production in barley (Hordeum vulgare L.). Nature 225:874-886). For example, wheat haploids can be obtained by pollination with various intergeneric crosses with maize (Suenaga and Nakajima 1989), pearl millet (Inagaki and Mujeeb-Kazi 1995), teosinte (Ushiyama et al. 1991), H. bulbosum (Barclay 1975), and sorghum (Ohkawa et al. 1992). Barley haploids are obtained by pollination with Hordeum bulbosum pollen. Tobacco haploids can be obtained by crossing with N. africana pollen. Many other examples exist in other crops.

Similar to examples above in introducing transgenic editing locus into Stock6 induction line, transgenic editing locus can be introduced into these lines used for wide crosses to induce haploid induction and targeted sequence mutation. Transgenic lines expressing editing machinery can be generated in any line of corn, wheat, barley, rye, pearl millet, rice, brassica, lettuce, tomato, or any other crop by direct transformation or outcrossing. Preferably the transgenic locus is made homozygous and then the line is used as pollen donor in a wide cross with other compatible recipient crops to induce haploids to produce desired edits. The process of post-fertilization genome elimination in wide crosses is basically the same as the process in the maize MATL mutant system, although in some cases the foreign pollen-derived DNA and editing machinery may be eliminated slightly earlier in embryo development, which is why this method is preferably practiced using a promoter that drives expression of the editing machinery in the pollen, sperm cells, and/or zygote cell, so that the editing RNA and protein is present and able to edit the target genome even though the male DNA is eliminated rather quickly after fertilization.

To demonstrate feasibility of simultaneous haploid induction and editing via wide crosses, maize transgenic lines expressing Cas9 and sgRNA targeting wheat VLHP gene sequences were generated. Vector 23763 (SEQ ID NO: 24) contains expression cassettes for Cas9 and sgRNA containing protospacer sequence xTaVLHP1 (5′-GACGAGCAGGCGCAGTTCC-3′, SEQ ID NO: 25) for guiding Cas9-mediated cleavage of TaVLHP1 target sites in wheat. The wheat genome has three xTaVLHP1 targets in total (TaVLHP1-4A, TaVLHP1-4B and TaVLHP1-4D), with each one in its three sub-genomes. The guide sequence in 23397 (SEQ ID NO: 20), xZmVLHP (5′-GCTGGAGCTGAGCTTCCGGG-3′, SEQ ID NO: 21) will also direct cleavage of wheat VLHP target sequences, xTaVLHP2-1A (5′-GCTGGAGCTGAGCTTCCGGG-3′, SEQ ID NO: 26) or xTaVLHP2-1B (5′-TCTGGAGCTGAGCTTCCGGG-3′, SEQ ID NO: 27). There are three VLHP2A genes containing xTaVLHP2-1A and 3 VLHP2B genes containing xTaVLHP2-1B sequences in the Chinese Spring wheat genome. Vectors 23397 and 23763 were transformed into maize inbred line NP2222 using Agrobacterium-mediated transformation to generate transgenic lines expressing Cas9 and sgRNA. Transgenic maize lines were grown in greenhouse and selfed to produce T1 plants.

Pollen collected from transgenic maize T0 or progeny T1 plants carrying T-DNA of vector 23397 or 23763 were used to pollinate emasculated spring wheat line AC-Nanda. At one to two days before anthesis, wheat florets were emasculated and two days later are pollinated with fresh maize pollen carrying the editing machinery. For convenience, spikelets from a Syngenta elite cytoplasmic male sterile (“CMS”) wheat line (16A300292) were also directly used as female donors to induce haploid embryo formation with transgenic maize pollen expressing 23397 or 23763 Cas9-sgRNA. Embryos were extracted from pollinated florets at 14-20 days after pollination for embryo rescue to recover haploid plantlets from the wheat×maize haploid induction system. Excised embryos were cultured on either full strength MS (Murashige and Skoog 1962) or ½ MS or B5 basal medium containing various modifications of organic supplements and grown in vitro for 3-5 weeks at 20-25° C. and 16-hour day length.

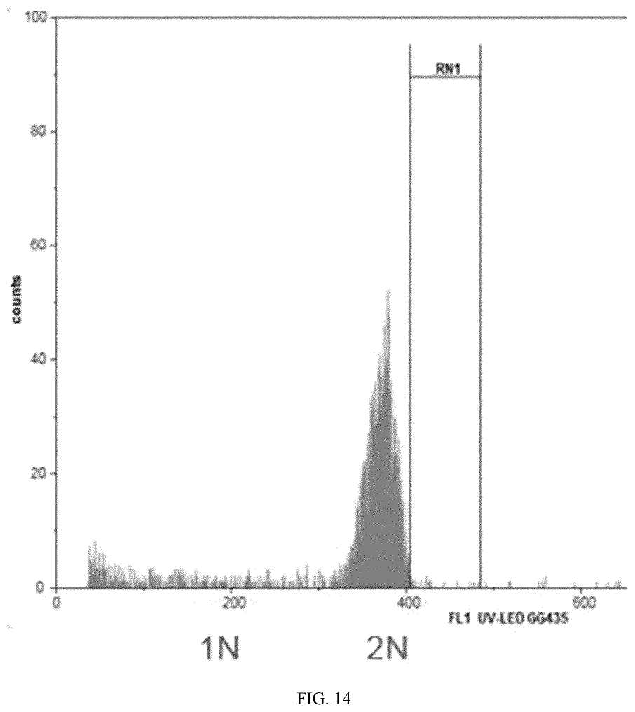

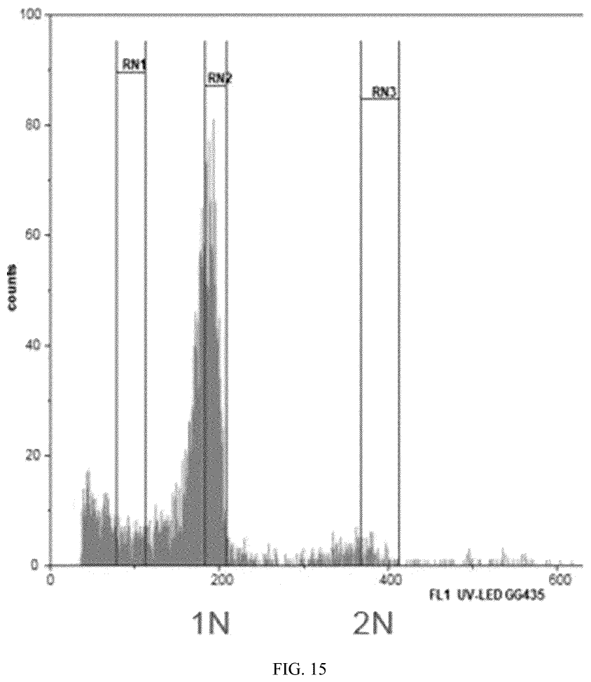

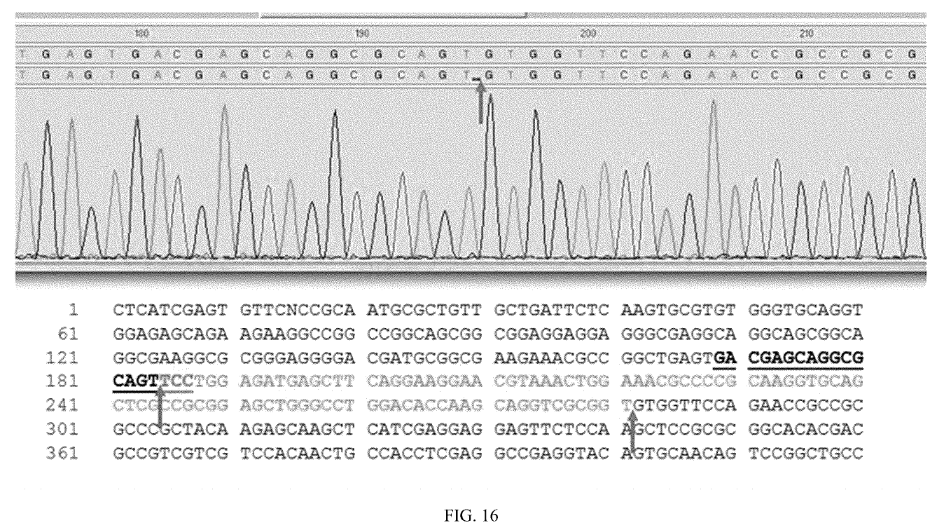

For example, pollen of T1 progeny from transgenic maize line MZET164902A044A containing vector 23763 was used to pollinate spikes of CMS line 16A300292 to induce wheat haploids. Haploid embryos were rescued and the resulting wheat haploid seedling were sampled for qPCR analysis to determine the copy number of VLHP target sequences (See Table 6). One of the haploid lines (JSWER30A22) was found to contain mutation in TaVLHP1-4B gene, but not in its orthologs TaVLHP1-4A and TaVLHP1-4D in the A and D sub-genomes. Ploidy level analysis confirmed that JSWER30A22 is a true haploid (See FIGS. 14 and 15). The mutation within the TaVLHP1-4B target region was further characterized by sequencing and was found to contain 97 bp deletion starting from the predicted Cas9 cleavage site (FIG. 16). We also identified another line JSW16A07 with “0” copy in TaVLHP1-4A gene (assay #3252), suggesting targeted editing in the target sequence. However, the deletion in this target gene is probably quite large in deleting the primer binding site(s) since we were not able to recover PCR product for sequencing. Haploid seedlings with an edited target site were transplanted to soil after 3-5 weeks in vitro culture. The transplanted seedlings were hardened for one week in a growth chamber under the same environmental regime. Colchicine was added after shoots had formed. However, the chromosome doubling treatment can be done earlier at embryo rescue in vitro culture stage or later after transplanting. When whole wheat seedlings are treated for doubling, roots of the haploid seedling are trimmed leaving a zone of 2-3 cm and then submerged in a 0.1% colchicine solution with 2% dimethyl sulfoxide (DMSO) and ca. 0.05% Tween-20 at 20° C. for 5 hours. After this treatment, the roots are washed to remove residual colchicine and potted in peat soil. Plant tissue samples can be removed from haploid seedlings for mutation detection to identify plants containing mutations in TaVLHP target gene sequences but with the maize chromosomes including sequences encoding the transgenic editing machinery completely eliminated. Since JSWER30A22 is from a CMS line, the plant is pollinated with a restorer to produce progeny seeds.

TABLE 6
Taqman analysis for wheat progeny from wide crosses. Line JSW30A22 is edited.

| | | Allele: | | | | |
|---|---|---|---|---|---|---|
| | | TAV_4A | TAV_4B | TAV_4D | PMI | CAS9 |
| | | Assay ID: | | | | |
| | | 3252 | 3253 | 3254 | 1750 | 2540 |
| | Construct | Copy# | Copy# | Copy# | Copy# | Copy# |
| Plant ID | ID | level | level | level | level | level |
| WT, AC-Nanda | N/A | >2 | 2 | >2 | 0 | 0 |
| WT, AC-Nanda | N/A | 2 | 2 | 2 | 0 | 0 |
| WT, CMS | N/A | 2 | 2 | 2 | 0 | 0 |
| WT, CMS | N/A | 2 | 2 | 2 | 0 | 0 |
| JSW29A01 | 23763 | 2 | 2 | 2 | 0 | 0 |
| JSW29A02 | 23763 | 2 | 2 | 2 | 0 | 0 |
| JSW29A03 | 23763 | 2 | 2 | 2 | 0 | 0 |
| JSW29A04 | 23763 | 2 | 2 | 2 | 0 | 0 |
| JSW29A05 | 23763 | 1 or 2 | 2 | 2 | 0 | 0 |
| JSW29A06 | 23763 | 2 | 2 | 2 | 0 | 0 |
| JSW29A07 | 23763 | 2 | 2 | 2 | 0 | 0 |
| JSW29A08 | 23763 | 2 | 2 | 2 | 0 | 0 |
| JSW29A09 | 23763 | 2 | 2 | 2 | 0 | 0 |
| JSW29A10 | 23763 | 2 | 2 | 2 | 0 | 0 |
| JSW29A11 | 23763 | 2 | 2 | 2 | 0 | 0 |
| JSW29A12 | 23763 | 2 | 2 | 2 | 0 | 0 |
| JSW29A13 | 23763 | 2 | 2 | 2 | 0 | 0 |
| JSW29A14 | 23763 | 2 | 2 | 2 | 0 | 0 |
| JSW29A15 | 23763 | 2 | 2 | 2 | 0 | 0 |
| JSW29A16 | 23763 | 2 | 2 | 2 | 0 | 0 |
| JSW30A01 | 23763 | 2 | 2 | 2 | 0 | 0 |
| JSW30A02 | 23763 | 2 | 1 or 2 | 2 | 0 | 0 |
| JSW30A03 | 23763 | 2 | 2 | 2 | 0 | 0 |
| JSW30A04 | 23763 | 2 | 2 | 2 | 0 | 0 |
| JSW30A05 | 23763 | 2 | 2 | 2 | 0 | 0 |
| JSW30A06 | 23763 | 2 | 2 | 2 | 0 | 0 |
| JSW30A07 | 23763 | 2 | 2 | 2 | 0 | 0 |
| JSW30A08 | 23763 | 2 | 2 | 2 | 0 | 0 |
| JSW30A09 | 23763 | 2 | 2 | 2 | 0 | 0 |
| JSW30A10 | 23763 | 2 | 2 | 2 | 0 | 0 |
| JSW30A11 | 23763 | 2 | 2 | 2 | 0 | 0 |
| JSW30A12 | 23763 | 2 | 2 | 2 | 0 | 0 |
| JSW30A13 | 23763 | 2 | 2 | 2 | 0 | 0 |
| JSW30A14 | 23763 | 2 | 2 | 2 | 0 | 0 |
| JSW30A15 | 23763 | 2 | 2 | 2 | 0 | 0 |
| JSW30A16 | 23763 | 2 | 2 | 2 | 0 | 0 |
| JSW30A17 | 23763 | 2 | 2 | 2 | 0 | 0 |
| JSW30A18 | 23763 | 2 | 2 | 2 | 0 | 0 |
| JSW30A19 | 23763 | >2 | 2 | 2 | 0 | 0 |
| JSW30A20 | 23763 | 2 | 2 | 2 | 0 | 0 |
| JSW30A21 | 23763 | 2 | 2 | 2 | 0 | 0 |
| JSW30A22 | 23763 | 2 | 0 | 2 | 0 | 0 |
| JSW30A23 | 23763 | 2 | 2 | 1 or 2 | 0 | 0 |
| JSW30A24 | 23763 | 2 | 2 | 2 | 0 | 0 |
| JSW30A25 | 23763 | 2 | 2 | 2 | 0 | 0 |
| JSW30A26 | 23763 | 2 | 2 | 2 | 0 | 0 |
| JSW30A27 | 23763 | 2 | 2 | 2 | 0 | 0 |
| JSW30A28 | 23763 | 2 | 2 | 2 | 0 | 0 |
| JSW30A29 | 23763 | 2 | 2 | 2 | 0 | 0 |
| JSW30A30 | 23763 | 2 | 1 or 2 | 1 or 2 | 0 | 0 |
| JSW30A31 | 23763 | 2 | 2 | 2 | 0 | 0 |

To further demonstrate feasibility of simultaneous haploid induction and editing via wide crosses, maize transgenic lines expressing Cas9 from five promoters that have high and/or specific expression in pollen, along with sgRNA targeting wheat VLHP gene sequences, were generated. These five vectors were 24038 (SEQ ID NO: 34), 24039 (SEQ ID NO: 35), 24079 (SEQ ID NO: 36), 24091 (SEQ ID NO: 37), and 24094 (SEQ ID NO: 38). All five of these vectors utilized the same sgRNA containing protospacer sequence xTaVLHP2 (5′-GCTGGAGCTGAGCTTCCGGG-3′, SEQ ID NO: 21) for guiding Cas9-mediated cleavage of TaVLHP2 target sites in wheat. The wheat genome has three xTaVLHP2 targets in total (TaVLHP2-2A, TaVLHP2-2B and TaVLHP2-2D), with each one in its three sub-genomes. The guide sequence in these five constructs also directs cleavage of wheat VLHP target sequences, xTaVLHP2 (5′-GCTGGAGCTGAGCTTCCGGG-3′, SEQ ID NO: 26) or xTaVLHP3 (5′-TCTGGAGCTGAGCTTCCGGG-3′, SEQ ID NO: 27). There are three TaVLHP2 genes containing xTaVLHP2 and 3 TaVLHP3 genes containing xTaVLHP2-1B sequences in the Chinese Spring wheat genome.

Vector 24038 (SEQ ID NO: 34) contains expression cassettes for Cas9 under control of a pollen-preferred high expression promoter prZmGRMZM5G876285 and terminator tZmGRMZM5G876285 from the maize prf3 (profilin homolog3) gene, which has an extremely high native expression at the RNA and protein level in pollen and which has evidence of high sperm cell expression.

Vector 24039 (SEQ ID NO: 35) contains expression cassettes for Cas9 under control of a pollen-preferred high expression promoter prZmGRMZM2G020852 and terminator tZmGRMZM2G020852 from the maize EXPB2 (BETA EXPANSIN2) gene, which has an extremely high native expression at the RNA and protein level in pollen and which has evidence of sperm cell expression.

Vector 24079 (SEQ ID NO: 36) contains expression cassettes for Cas9 under control of a pollen-preferred high expression promoter prZmGRMZM2G146551 and terminator tZmGRMZM2G146551 from the maize EXPB1 (BETA EXPANSIN1) gene, which has an extremely high native expression at the RNA and protein level in pollen and which has evidence of sperm cell expression.

Vector 24091 (SEQ ID NO: 37) contains expression cassettes for Cas9 under control of a pollen-preferred promoter prZmGRMZM2G471240 and terminator tZmGMRMZM2G471240 from the maize MATL (MATRILINEAL) gene, which shows evidence of pollen and possibly sperm cell expression at the RNA and protein level.

Vector 24094 (SEQ ID NO: 38) contains expression cassettes for Cas9 under control of a pollen-preferred promoter prZmGRMZM2G471240 and terminator tZmGMRMZM2G471240 from the maize MATL (MATRILINEAL) gene, which shows evidence of pollen and possibly sperm cell expression at the RNA and protein level. This construct additionally has an N-terminal fusion of AmCyan fluorescent protein on the Cas9 molecule for imaging and visualization of the Cas9 localization in pollen.

These five vectors (24038, 24039, 24079, 24091, and 24094) were transformed into maize inbred line NP2222 using Agrobacterium-mediated transformation to generate transgenic lines expressing Cas9 and sgRNA.

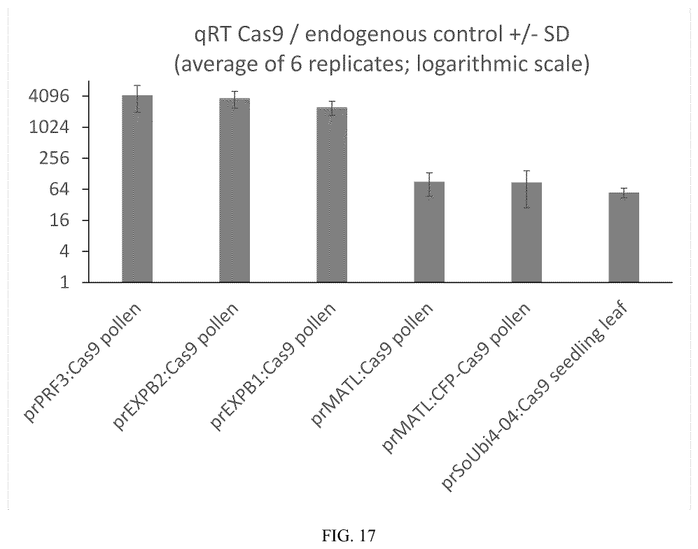

Transgenic maize lines were grown in greenhouse and single and two-copy transgenic plants were outcrossed onto spring wheat and a CMS wheat line. Pollen collected from transgenic maize T0 plants carrying T-DNAs of one of the vectors 24038, 24039, 24079, 24091, and 24094 were used to pollinate emasculated spring wheat line AC-Nanda. Pollen was also used for a qRT experiment, in which the expression of the Cas9 was measured at the RNA level and compared to Cas9 expression in leaf samples when the Cas9 was driven by a sugar cane ubiquitin promoter used in many of the corn and wheat examples given above. As you can see in FIG. 17, the expression was high in the pollen, averaging about 100 fold higher in plants carrying the T-DNA vectors 24038, 24039, and 24079 compared to the Ubiquitin promoter. The expression was also higher in pollen from plants containing vectors 24038, 24039, and 24079 when compared to the pollen carrying the MATRILINEAL promoter constructs (24094 and 24091), which is known to have lower native gene expression. All five of these promoters have expression patterns that are restricted to pollen. As an indication that the promoters were working properly, we observed no T0 expression of Cas9 in callus seedling leaves, and there was no editing of the VLHP target sites in the T0 maize leaves (without wishing to be bound by theory, editing may happen at the maize target sites, in all likelihood, at the mature pollen stage, when the Cas9 is expressed for the first time).

At one to two days before anthesis, wheat florets were emasculated from the CMS line and the AC Nanda line. Two days later the florets were pollinated with fresh maize pollen carrying the editing machinery, Cas9-sgRNA, from either construct 24038, 24039, 24091, or 24094 (T0 plants transformed with construct 24079 were delayed, and not crossed to wheat in this manner). Wheat embryos were extracted from pollinated florets at 14-20 days after pollination for embryo rescue to recover haploid plantlets from the wheat×maize haploid induction system. Excised embryos were cultured on either full strength MS (Murashige and Skoog 1962) or ½ MS or B5 basal medium containing various modifications of organic supplements and grown in vitro for 1-5 weeks at 20-25° C. and 16-hour day length. For example, pollen of T0 progeny from transgenic maize line MZKE172601A100A containing vector 24039 was used to pollinate spikes of CMS line 16A300292 to induce wheat haploids. Haploid embryos were rescued and the resulting wheat haploid seedlings were sampled for qPCR analysis to determine the copy number of VLHP target sequences (Table 7). In this analysis, we tested for the Cas9 transgene using assay #2540. All wheat embryos rescued and tested lacked this transgene and gave scores of “0” for Cas9, because they do not have any corn DNA in the developing embryo and therefore do not have the transgene. The corn DNA is totally eliminated, kicked out or fails to be fully delivered in the first place during the haploid induction process, taking place during and/or after fertilization). In addition to Cas9, we test for assays #3332 and #3333, which give non-specific amplification of both VLHP2-2A and -2D alleles. These assays typically read as “2” or “>2” in haploid wheat, and the majority of the haploids we produced using the transgenic maize pollen scored 2 or >2 for these assays. We used these assays to look for putative edited haploids, by looking for scores of 0 or 1. A call of “1” might indicate that one of the two alleles, either VLHP2-2A, or -2D, was edited. Finally, we tested for assay 3255 in AC Nanda haploids, which detects VLHP2-2B specifically. The CMS line does not amplify this assay, even when it is wild-type, so we did not use it for the CMS haploids. The unedited haploids give a score of a “2,” while putative edited haploids are found because they have a score of “0.” A score of “1” might indicate a faulty reading or a chimeric, partially-edited sample.

As an example, one of the AC Nanda haploid plants 440-A5 was found to contain mutation in TaVLHP2-2B gene, but not in its orthologs TaVLHP2-2A and TaVLHP2-2D in the A and D sub-genomes (Table 7). The Taqman data also showed that it lacked the Cas9 transgene. The mutation within the TaVLHP2-2B target region was further characterized by sequencing, but although we were able to amplify the A and D alleles, we could no longer amplify the B allele, suggesting that there is a larger edit present, likely a large deletion, that results in the PCR product no longer amplifying.

As another example, one of the CMS haploid plants 450-D11 was found to contain mutation in either the TaVLHP2-2D or -2A homologues, according to the score of “1” for both assays 3332 and 3333. (Table 7). The taqman data showed that it lacked the Cas9 transgene. The TaVLHP2-2A, 2B and 2D target regions were further characterized by sequencing, but although we were able to amplify the A and B alleles, we could no longer amplify the D allele, suggesting that there is a larger edit present that led to PCR failure.

Considering the 2295 wheat haploids produced from crosses to maize pollen carrying one of the following five preferred-pollen expression constructs (24038, 24039, 24091, and 24094), we found 15 haploids that gave Taqman assay data that indicated possible editing at either the VLHP2-2A, VLHP2-2D, or VLHP2-2B target sites. After sequencing, seven of those haploids were found to have wild-type sequences at the target sites, and were called false positives due to Taqman error. These errors are thought to be either due to the fact that assays #3332 and #3333 gave non-specific amplification of both VLHP-2A and -2D alleles, leading to some missed calls, or due to low DNA quantity.

Of the remaining 8 putative edited haploids, six were AC Nanda (440-B3, 440-D3, 440-A5, 447-G8, 456-G9, 459-A2) where the editing transgene was from construct 24038. Four of those (440-B3, 440-D3, 440-A5, and 456-G9) contained edits in VLHP2-2B. These were found because they had a Taqman score of “0” for assay 3255. These plants lacked Cas9 (score of “0”) but had wild-type “2” scores for VLHP2-2A or VLHP2-2D (assays #3332 and #3333) indicating they were not edited that those sites. These six plants were confirmed to be haploids by ploidy analysis. We attempted to sequence the edited alleles, but while the PCR and sequencing reactions worked well for 2A and 2D, we were not able to obtain a PCR product for 2B. We repeated the PCR several times using a range of reaction conditions but could not amplify the 2B homologs from these haploid plants. This may indicate that the editing caused a large change in the 2B gene in these plants that may end up deleting the primer annealing site. We expect that many of the CMS plants also have edits at the VLHP2-2B target site, but we did not have an assay to detect the VLHP2-2B allele from the CMS line.

Considering AC Nanda alone, we calculate an overall editing rate at that allele of 0.7% for all constructs, but a particularly high editing rate of 1.4% for construct 24038.

In addition to these four edited haploids with scores of “0” for 3255, several other plants gave scores of “0 or 1” or “1” for 3255, which indicates possible chimerism (partial editing in certain cell lineages of the embryo or plantlet), but we did not follow up on those plants. For AC Nanda homolog VLHP2-2A, plant 447-G8 contained an edit which we were also not able to sequence because the PCR reaction failed, even though 2B and 2D did amplify and contained wild-type sequence. We repeated the PCR several times using a range of reaction conditions but could not amplify the 2A homolog. Similarly, for VLHP2-2D, plant 459-A2 contained an edit which we were not able to sequence because the PCR reaction failed. We repeated the PCR several times using a range of reaction conditions but could not amplify the 2D homolog. We also found putative edits in 447-H12 and 440-G6, but upon sequencing we found that these were false positives.

For the CMS haploids, plant 450-D11 gave scores of “1” for both assay #3332 and 3333 (Table 7). Upon sequencing, we found that the 2A homolog had wild-type sequence, but we could not PCR-amplify the 2D homolog, suggesting that a large edit had occurred. We repeated the PCR several times using a range of reaction conditions but could not amplify the 2D homolog. For plant 452-B11, the Taqman score was “0” for #3332 (VLHP2-2A), and we could not amplify that allele for sequencing, even though the 2D and 2B PCR products and sequences were normal. We repeated the PCR several times using a range of reaction conditions but could not amplify the 2A homolog. We also found five plants that had putative edits according to the Taqman data for assays 3332 and 3333, but PCR-sequencing showed these to be false positives; the sequence was wild-type (unedited).

In total, we found two edited CMS haploids and six edited AC Nanda haploids. There may be many more edited haploids that we were not able to detect because we did not have assays for the 2B gene for the CMS plants, nor for the VLHP3 gene target sites of the guide RNA in these five constructs.

The sequencing data from these edited haploids are consistent with the concept of a large deletion, inversion or rearrangement around the guide RNA target site, and extending far enough away to possibly include removal of one of the primer binding sites. This type of large change is not uncommon during editing by Cas9, especially in tissues where DNA repair via non-homologous end-joining is slower or inhibited—which may be the case in the just-fertilized zygote or early haploid wheat embryo.

TABLE 7
Sequencing data from edited wheat haploids.

| | | | TAV_2A | | TAV_2D | | TAV_2B | | PMI | | Cas9 | | |
|---|---|---|---|---|---|---|---|---|---|---|---|---|---|
| | | | 3332 | | 3333 | | 3255 | | 1750 | | 2540 | | |
| | | | Raw | Copy | Raw | Copy | Raw | Copy | Raw | Copy | Raw | Copy | |
| | Construct | copy | Copy | # | Copy | # | Copy | # | Copy | # | Copy | # | Sequencing |
| Plant ID | ID | # | # | level | # | level | # | level | # | level | # | level | result |
| CMS | | | | | | | | | | | | | |
| 427-A2 | WT | N/A | 2.44 | >2 | 2.38 | 2 | Not tested | | 0.00 | 0 | 0.00 | 0 | not sequenced |
| 427-B2 | WT | N/A | 1.99 | 2 | 1.99 | 2 | Not tested | | 0.00 | 0 | 0.00 | 0 | not sequenced |
| 427-C2 | WT | N/A | 2.02 | 2 | 2.07 | 2 | Not tested | | 0.00 | 0 | 0.00 | 0 | not sequenced |
| 427-D2 | WT | N/A | 2.31 | 2 | 2.16 | 2 | Not tested | | 0.00 | 0 | 0.00 | 0 | not sequenced |
| 427-A10 | 24091 | 2 | 2.07 | 2 | 1.66 | 2 | Not tested | | 0.00 | 0 | 0.00 | 0 | not sequenced |
| 427-B10 | 24091 | 2 | 1.95 | 2 | 1.84 | 2 | Not tested | | 0.00 | 0 | 0.00 | 0 | not sequenced |
| 427-C10 | 24091 | 2 | 1.93 | 2 | 2.28 | 2 | Not tested | | 0.00 | 0 | 0.00 | 0 | not sequenced |
| 427-D10 | 24091 | 2 | 2.59 | >2 | 2.48 | >2 | Not tested | | 0.00 | 0 | 0.00 | 0 | not sequenced |
| 427-E10 | 24091 | 2 | 1.90 | 2 | 1.78 | 2 | Not tested | | 0.00 | 0 | 0.00 | 0 | not sequenced |
| 427-F10 | 24091 | 2 | 2.03 | 2 | 1.96 | 2 | Not tested | | 0.00 | 0 | 0.00 | 0 | not sequenced |
| 427-G10 | 24091 | 2 | 2.08 | 2 | 2.25 | 2 | Not tested | | 0.00 | 0 | 0.00 | 0 | not sequenced |
| 427-H10 | 24091 | 2 | 0.58 | 1 | 0.81 | 1 | Not tested | | 0.00 | 0 | 0.00 | 0 | A and D were |
| | | | | | | | | | | | | | both WT |
| 427-A11 | 24091 | 2 | 1.57 | 1 or 2 | 1.93 | 2 | Not tested | | 0.00 | 0 | 0.00 | 0 | not sequenced |
| 427-B11 | 24091 | 2 | 1.41 | 1 or 2 | 1.63 | 2 | Not tested | | 0.00 | 0 | 0.00 | 0 | not sequenced |
| 427-C11 | 24091 | 2 | 1.06 | 1 | 1.21 | 1 | Not tested | | 0.01 | 0 | 0.01 | 0 | not sequenced |
| 427-D11 | 24091 | 2 | 1.98 | 2 | 2.03 | 2 | Not tested | | 0.00 | 0 | 0.00 | 0 | not sequenced |
| 427-E11 | 24091 | 2 | 1.94 | 2 | 1.94 | 2 | Not tested | | 0.00 | 0 | 0.00 | 0 | not sequenced |
| 427-F11 | 24091 | 2 | 1.84 | 2 | 1.84 | 2 | Not tested | | 0.00 | 0 | 0.00 | 0 | not sequenced |
| 427-G11 | 24091 | 2 | 1.54 | 1 or 2 | 1.76 | 2 | Not tested | | 0.00 | 0 | 0.00 | 0 | not sequenced |
| 427-H11 | 24091 | 2 | 1.75 | 2 | 1.76 | 2 | Not tested | | 0.00 | 0 | 0.00 | 0 | not sequenced |
| 427-A12 | 24091 | 2 | 1.99 | 2 | 2.15 | 2 | Not tested | | 0.00 | 0 | 0.00 | 0 | not sequenced |
| 427-B12 | 24091 | 2 | 0.72 | 1 | 1.26 | 1 | Not tested | | 0.00 | 0 | 0.00 | 0 | A and D were |
| | | | | | | | | | | | | | both WT |
| 427-C12 | 24091 | 2 | 1.69 | 2 | 1.50 | 1 or 2 | Not tested | | 0.00 | 0 | 0.01 | 0 | not sequenced |
| 427-D12 | 24091 | 1 | 2.34 | 2 | 2.03 | 2 | Not tested | | 0.00 | 0 | 0.00 | 0 | not sequenced |
| 427-E12 | 24091 | 1 | 1.98 | 2 | 2.04 | 2 | Not tested | | 0.00 | 0 | 0.00 | 0 | not sequenced |
| 427-F12 | 24091 | 1 | 1.89 | 2 | 1.97 | 2 | Not tested | | 0.00 | 0 | 0.00 | 0 | not sequenced |
| 427-G12 | 24091 | 1 | 1.56 | 1 or 2 | 1.77 | 2 | Not tested | | 0.00 | 0 | 0.00 | 0 | not sequenced |
| 427-H12 | 24091 | 1 | 1.57 | 1 or 2 | 2.36 | 2 | Not tested | | 0.00 | 0 | 0.00 | 0 | not sequenced |
| 428-A3 | 24091 | 1 | 2.12 | 2 | 1.75 | 2 | Not tested | | 0.00 | 0 | 0.00 | 0 | not sequenced |
| 428-B3 | 24091 | 1 | 2.69 | >2 | 1.89 | 2 | Not tested | | 0.00 | 0 | 0.00 | 0 | not sequenced |
| 428-C3 | 24091 | 1 | 2.09 | 2 | 2.44 | >2 | Not tested | | 0.00 | 0 | 0.00 | 0 | not sequenced |
| 428-D3 | 24091 | 1 | 2.05 | 2 | 2.39 | 2 | Not tested | | 0.00 | 0 | 0.00 | 0 | not sequenced |
| 428-E3 | 24091 | 1 | 2.48 | >2 | 2.87 | >2 | Not tested | | 0.00 | 0 | 0.00 | 0 | not sequenced |
| 428-F3 | 24091 | 1 | 2.33 | 2 | 2.76 | >2 | Not tested | | 0.00 | 0 | 0.00 | 0 | not sequenced |
| 428-G3 | 24091 | 1 | 2.84 | >2 | 0.22 | 0 | Not tested | | 0.00 | 0 | 0.00 | 0 | A and D were |
| | | | | | | | | | | | | | both WT |
| 428-H3 | 24091 | 1 | 2.83 | >2 | 2.60 | >2 | Not tested | | 0.00 | 0 | 0.00 | 0 | not sequenced |
| 450-A11 | 24094 | 1 | 1.97 | 2 | 2.24 | 2 | Not tested | | 0.00 | 0 | 0.00 | 0 | not sequenced |
| 450-B11 | 24094 | 1 | 2.13 | 2 | 2.04 | 2 | Not tested | | 0.00 | 0 | 0.00 | 0 | not sequenced |
| 450-C11 | 24094 | 1 | 2.15 | 2 | 2.18 | 2 | Not tested | | 0.00 | 0 | 0.00 | 0 | not sequenced |
| 450-D11 | 24094 | 1 | 1.04 | 1 | 0.99 | 1 | Not tested | | 0.00 | 0 | 0.00 | 0 | A & B were WT; |
| | | | | | | | | | | | | | D failed |
| 450-E11 | 24094 | 1 | 2.35 | 2 | 2.01 | 2 | Not tested | | 0.00 | 0 | 0.00 | 0 | not sequenced |
| 450-F11 | 24094 | 1 | 2.02 | 2 | 1.90 | 2 | Not tested | | 0.00 | 0 | 0.00 | 0 | not sequenced |
| 450-G11 | 24039 | 1 | 1.76 | 2 | 1.72 | 2 | Not tested | | 0.00 | 0 | 0.00 | 0 | not sequenced |
| 450-H11 | 24039 | 1 | 2.07 | 2 | 2.04 | 2 | Not tested | | 0.00 | 0 | 0.00 | 0 | not sequenced |
| 452-H4 | 24038 | 2 | 2.62 | >2 | 0.01 | 0 | Not tested | | 0.00 | 0 | 0.00 | 0 | A and D were |
| | | | | | | | | | | | | | both WT |
| 452-A11 | 24038 | 2 | 2.24 | 2 | 2.28 | 2 | Not tested | | 0.00 | 0 | 0.00 | 0 | not sequenced |
| 452-B11 | 24038 | 2 | 0.00 | 0 | 2.22 | 2 | Not tested | | 0.00 | 0 | 0.00 | 0 | B & D were WT; |
| | | | | | | | | | | | | | A failed |
| 452-C11 | 24038 | 2 | 2.55 | >2 | 2.22 | 2 | Not tested | | 0.00 | 0 | 0.00 | 0 | not sequenced |
| 452-D11 | 24038 | 2 | 0.82 | 1 | 1.26 | 1 | Not tested | | 0.00 | 0 | 0.00 | 0 | A and D were |
| | | | | | | | | | | | | | both WT |
| 452-E11 | 24038 | 2 | 2.43 | >2 | 2.36 | 2 | Not tested | | 0.00 | 0 | 0.00 | 0 | not sequenced |
| 452-F11 | 24038 | 2 | 2.12 | 2 | 2.21 | 2 | Not tested | | 0.00 | 0 | 0.00 | 0 | not sequenced |
| 452-G11 | 24038 | 2 | 2.38 | 2 | 1.99 | 2 | Not tested | | 0.00 | 0 | 0.00 | 0 | not sequenced |
| 452-H11 | 24038 | 2 | 1.82 | 2 | 1.83 | 2 | Not tested | | 0.00 | 0 | 0.00 | 0 | not sequenced |
| NANDA | | | | | | | | | | | | | |
| 425-A2 | WT | N/A | 2.30 | 2 | 2.62 | >2 | 1.908 | 2 | 0.00 | 0 | 0.00 | 0 | not sequenced |
| 425-B2 | WT | N/A | 2.28 | 2 | 2.41 | >2 | 2.274 | 2 | 0.00 | 0 | 0.00 | 0 | not sequenced |
| 425-C2 | WT | N/A | 2.47 | >2 | 1.92 | 2 | 1.962 | 2 | 0.00 | 0 | 0.00 | 0 | not sequenced |
| 425-D2 | WT | N/A | 2.10 | 2 | 2.11 | 2 | 1.772 | 2 | 0.00 | 0 | 0.00 | 0 | not sequenced |
| 447-A12 | 24038 | 2 | 1.72 | 2 | 1.90 | 2 | 2.02 | 2 | 0.00 | 0 | 0.00 | 0 | not sequenced |
| 447-B12 | 24039 | 2 | 2.18 | 2 | 1.62 | 2 | 1.47 | 1 or 2 | 0.00 | 0 | 0.00 | 0 | not sequenced |
| 447-C12 | 24039 | 2 | 1.78 | 2 | 2.40 | 2 | 1.90 | 2 | 0.00 | 0 | 0.00 | 0 | not sequenced |
| 447-D12 | 24039 | 2 | 1.58 | 1 or 2 | 1.70 | 2 | 2.18 | 2 | 0.00 | 0 | 0.00 | 0 | not sequenced |
| 447-E12 | 24039 | 2 | 2.13 | 2 | 1.82 | 2 | 2.14 | 2 | 0.00 | 0 | 0.00 | 0 | not sequenced |
| 447-F12 | 24039 | 2 | 2.25 | 2 | 1.78 | 2 | 2.15 | 2 | 0.00 | 0 | 0.00 | 0 | not sequenced |
| 447-G12 | 24039 | 2 | 1.90 | 2 | 2.30 | 2 | 2.23 | 2 | 0.00 | 0 | 0.00 | 0 | not sequenced |
| 447-H12 | 24039 | 1 | 2.34 | 2 | 1.95 | 2 | 0.89 | 1 | 0.00 | 0 | 0.00 | 0 | A, B, and D |
| | | | | | | | | | | | | | were all WT |
| 440-A2 | 24039 | 1 | 1.72 | 2 | 1.71 | 2 | 1.24 | 1 | 0.00 | 0 | 0.00 | 0 | not sequenced |
| 440-B2 | 24039 | 1 | 2.30 | 2 | 2.56 | >2 | 1.77 | 2 | 0.00 | 0 | 0.00 | 0 | not sequenced |
| 440-C2 | 24039 | 1 | 3.05 | >2 | 1.85 | 2 | 2.01 | 2 | 0.00 | 0 | 0.00 | 0 | not sequenced |
| 440-D2 | 24039 | 1 | 1.66 | 2 | 1.70 | 2 | 1.44 | 1 or 2 | 0.00 | 0 | 0.00 | 0 | not sequenced |
| 440-E2 | 24039 | 1 | 2.23 | 2 | 1.91 | 2 | 2.04 | 2 | 0.00 | 0 | 0.00 | 0 | not sequenced |
| 440-F2 | 24039 | 1 | 2.63 | >2 | 2.07 | 2 | 2.28 | 2 | 0.00 | 0 | 0.00 | 0 | not sequenced |
| 440-G2 | 24038 | 11 | 1.91 | 2 | 1.87 | 2 | 2.10 | 2 | 0.00 | 0 | 0.00 | 0 | not sequenced |
| 440-H2 | 24038 | 1 | 1.85 | 2 | 1.80 | 2 | 1.97 | 2 | 0.00 | 0 | 0.00 | 0 | not sequenced |
| 440-A3 | 24038 | 1 | 2.52 | >2 | 2.05 | 2 | 1.99 | 2 | 0.00 | 0 | 0.00 | 0 | not sequenced |
| 440-B3 | 24038 | 1 | 2.16 | 2 | 2.19 | 2 | 0.00 | 0 | 0.00 | 0 | 0.00 | 0 | A & D were WT; |
| | | | | | | | | | | | | | B failed |
| 440-C3 | 24038 | 1 | 2.58 | >2 | 2.02 | 2 | 2.78 | >2 | 0.00 | 0 | 0.00 | 0 | not sequenced |
| 440-D3 | 24038 | 1 | 2.34 | 2 | 2.32 | 2 | 0.00 | 0 | 0.00 | 0 | 0.00 | 0 | A & D were WT; |
| | | | | | | | | | | | | | B failed |
| 440-E3 | 24038 | 1 | 2.49 | >2 | 2.23 | 2 | 2.47 | >2 | 0.00 | 0 | 0.00 | 0 | not sequenced |
| 440-F3 | 24038 | 1 | 2.08 | 2 | 2.10 | 2 | 2.17 | 2 | 0.00 | 0 | 0.00 | 0 | not sequenced |
| 440-F4 | 24038 | 1 | 1.73 | 2 | 1.47 | 1 or 2 | 1.41 | 1 or 2 | 0.00 | 0 | 0.00 | 0 | not sequenced |
| 440-G4 | 24038 | 1 | 1.53 | 1 or 2 | 2.02 | 2 | 1.99 | 2 | 0.00 | 0 | 0.00 | 0 | not sequenced |
| 440-H4 | 24038 | 1 | 2.22 | 2 | 1.90 | 2 | 0.00 | 0 | 0.00 | 0 | 0.00 | 0 | not sequenced |
| 440-A5 | 24038 | 1 | 2.22 | 2 | 1.90 | 2 | 0.00 | 0 | 0.00 | 0 | 0.00 | 0 | A & D were WT; |
| | | | | | | | | | | | | | B failed |
| 440-A6 | 24039 | 2 | 2.49 | >2 | 2.32 | 2 | 1.84 | 2 | 0.00 | 0 | 0.00 | 0 | not sequenced |
| 440-B6 | 24039 | 2 | 2.12 | 2 | 2.03 | 2 | 2.21 | 2 | 0.00 | 0 | 0.00 | 0 | not sequenced |
| 440-C6 | 24039 | 2 | 2.63 | >2 | 2.07 | 2 | 2.28 | 2 | 0.00 | 0 | 0.00 | 0 | not sequenced |
| 440-D6 | 24039 | 2 | 2.49 | >2 | 2.23 | 2 | 2.47 | >2 | 0.00 | 0 | 0.00 | 0 | not sequenced |
| 440-E6 | 24039 | 2 | 2.45 | >2 | 2.20 | 2 | 2.32 | 2 | 0.00 | 0 | 0.00 | 0 | not sequenced |
| 440-F6 | 24039 | 2 | 2.10 | 2 | 1.92 | 2 | 1.91 | 2 | 0.00 | 0 | 0.00 | 0 | not sequenced |
| 440-G6 | 24039 | 2 | 0.57 | 1 | 0.66 | 1 | 0.53 | 1 | 0.00 | 0 | 0.00 | 0 | A, B & D were |
| | | | | | | | | | | | | | all WT |
| 440-H6 | 24039 | 2 | 1.81 | 2 | 1.96 | 2 | 2.51 | >2 | 0.00 | 0 | 0.00 | 0 | not sequenced |
| 447-A8 | 24038 | 1 | 2.42 | >2 | 2.21 | 2 | 2.10 | 2 | 0.00 | 0 | 0.00 | 0 | not sequenced |
| 447-B8 | 24038 | 1 | 2.46 | >2 | 2.32 | 2 | 2.09 | 2 | 0.00 | 0 | 0.00 | 0 | not sequenced |
| 447-C8 | 24038 | 1 | 2.09 | 2 | 2.08 | 2 | 2.29 | 2 | 0.00 | 0 | 0.00 | 0 | not sequenced |
| 447-D8 | 24038 | 1 | 2.13 | 2 | 2.14 | 2 | 2.34 | 2 | 0.00 | 0 | 0.00 | 0 | not sequenced |
| 447-E8 | 24038 | 11 | 2.36 | 2 | 2.31 | 2 | 2.44 | >2 | 0.00 | 0 | 0.00 | 0 | not sequenced |
| 447-F8 | 24038 | 1 | 2.72 | >2 | 2.28 | 2 | 2.00 | 2 | 0.00 | 0 | 0.00 | 0 | not sequenced |
| 447-G8 | 24038 | 1 | 0.71 | 1 | 1.34 | 1 or 2 | 2.33 | 2 | 0.00 | 0 | 0.00 | 0 | B & D were WT; |
| | | | | | | | | | | | | | A failed |
| 447-H8 | 24038 | 1 | 2.25 | 2 | 2.29 | 2 | 2.01 | 2 | 0.00 | 0 | 0.00 | 0 | not sequenced |
| 456-A9 | 24038 | 2 | 2.19 | 2 | 1.59 | 1 or 2 | 2.03 | 2 | 0.00 | 0 | 0.00 | 0 | not sequenced |
| 456-B9 | 24038 | 2 | 2.13 | 2 | 2.11 | 2 | 2.02 | 2 | 0.00 | 0 | 0.00 | 0 | not sequenced |
| 456-C9 | 24038 | 2 | 2.16 | 2 | 1.85 | 2 | 1.45 | 1 or 2 | 0.00 | 0 | 0.00 | 0 | not sequenced |
| 456-D9 | 24038 | 2 | 2.56 | >2 | 2.18 | 2 | 1.76 | 2 | 0.00 | 0 | 0.00 | 0 | not sequenced |
| 456-E9 | 24038 | 2 | 2.29 | 2 | 2.03 | 2 | 1.65 | 2 | 0.00 | 0 | 0.00 | 0 | not sequenced |
| 456-F9 | 24038 | 2 | 2.24 | 2 | 2.02 | 2 | 2.05 | 2 | 0.00 | 0 | 0.00 | 0 | not sequenced |
| 456-G9 | 24038 | 2 | 2.49 | >2 | 2.03 | 2 | 0.00 | 0 | 0.00 | 0 | 0.00 | 0 | A & D were WT; |
| | | | | | | | | | | | | | B failed |
| 456-H9 | 24038 | 2 | 1.78 | 2 | 1.62 | 2 | 1.38 | 1 or 2 | 0.00 | 0 | 0.00 | 0 | not sequenced |
| 459-A2 | 24038 | 2 | 1.38 | 1 or 2 | 1.11 | 1 | 0.94 | 1 | 0.00 | 0 | 0.00 | 0 | A & B were WT; |
| | | | | | | | | | | | | | D failed |
| 459-B2 | 24038 | 2 | 1.86 | 2 | 1.91 | 2 | 2.12 | 2 | 0.00 | 0 | 0.00 | 0 | not sequenced |
| 459-C2 | 24038 | 2 | 1.94 | 2 | 2.09 | 2 | 1.42 | 1 or 2 | 0.00 | 0 | 0.00 | 0 | not sequenced |
| 459-D2 | 24038 | 2 | 2.09 | 2 | 2.05 | 2 | 1.91 | 2 | 0.00 | 0 | 0.00 | 0 | not sequenced |
| 459-E2 | 24038 | 2 | 2.18 | 2 | 2.12 | 2 | 2.12 | 2 | 0.00 | 0 | 0.00 | 0 | not sequenced |

Overall, we found that the editing frequency (number of edited haploids identified divided by the total number of haploids) for construct 24038 was 0.79%; for construct 24039 it was 0%; for construct 24091 it was 0%, and for construct 24094 it was 0.75%. However, this editing rate is certainly an under-estimate because we did not have assays to detect edits at many of the guide RNA target sites. Additionally, because we used T0 pollen that was either 1 or 2 copy, we know that with the 1-copy pollen, only 50% of the fertilizing pollen grains will contain the Cas9, and so only half of the embryos have the opportunity to be edited; similarly, for 2 copy parents, assuming random segregation of the transgenes in the male meiosis, we would expect about 75% of the pollen to contain Cas9, so 25% of the embryos cannot be edited. It is reasonable to conclude that, when one is trying to use this simultaneous editing plus haploid induction technology with the editing machinery carried by the pollen, it may in some cases be more optimal to use a promoter that express specifically or highly in pollen and in sperm cells, so that the Cas9 can be expressed at a higher level. In cases where the gene target might impact development of the haploid inducer plant, having a pollen or sperm-preferred promoter that does not express in leaves might be useful because it would avoid editing the target gene in the haploid inducer plant during development—perhaps editing it for the first time in pollen.

Because the sperm cells fertilize the egg, they have the potential to deliver Cas9 RNA and protein (as well as the transgene DNA itself, integrated into one of the male chromosomes that will be eliminated). As we demonstrated in the wide-cross work in this example, it may work well to have the Cas9 and/or guide RNA under the control of a promoter that specifically or highly expresses in pollen, and in particular in sperm cells, when using a haploid inducer as the male to edit elite lines. We do not know exactly whether MATRILINEAL, EXPB1, EXPB2, and PRF3 express in the vegetative nucleus, the sperm cells, or both, and whether there might be any expression in a zygote cell type, but these were chosen because they are supposedly highly and/or specifically expressed in pollen. The PRF3 promoter has a DUO1 binding motif in the promoter, which may indicate it expresses in sperm cells. This is consistent with that promoter having higher editing frequency. The fact that we found many edited wheat haploids after the wide cross makes it clear that when there is high expression of Cas9 in pollen, using these or any other promoter, that expression can lead to editing in the wheat embryos after the wide cross. There is a strong possibility that these promoters, as well as other promoters that drive expression in pollen, or in particular in the sperm cells, might increase the efficiency of the editing process during corn haploid induction, or rice haploid induction.

Similarly, in the next example below, we show haploid editing in a dicot using a CENH3-modified-haploid inducer line, and we use constitutive promoter to drive the Cas9. But in an attempt to increase the efficiency of the haploid editing, we could opt to use a promoter that drives high and/or specific expression in egg cells, such as the EGG APPARATUS1 gene's promoter (“prEA1”) (see, e.g., Gray-Mitsumune, M. and Matton, D. P., The Egg apparatus 1 gene from maize is a member of a large gene family found in both monocots and dicots, PLANTA 223(3):618-625 (February 2006)) or EGG CELL1 (EC1) (see, e.g., Sprunck S, et al., Egg cell-secreted EC1 triggers sperm cell activation during double fertilization. Science 2012; 338:1093-97; PMID:23180860; http://dx.doi.org/10.1126/science.1223944).

As an example of this, one could use a sperm-cell expressed promoter, such as the Arabidopsis sperm-specific DUO1 promoter (see, e.g., Engel, et al., Green Sperm. Identification of Male Gamete Promoters in Arabidopsis, PLANT PHYSIOLOGY August 2005, 138 (4) 2124-2133; DOI: 10.1104/pp. 104.054213), or homologs of DUO1 from other species (for instance, the maize genes GRMZM2G105137 and GRMZM2G046443 are both DUO1 homologs that share a similar pollen-specific expression pattern). If one used any of these to drive Cas9 expression in the sperm cells of a haploid inducer line like RWK, NP2222-HI, or an matl mutant, it might make a highly efficient haploid editor line for use in editing diverse elite maize or wheat germplasm, via intraspecific or wide cross, respectively.

Other suitable sperm-expressed promoters for this concept of driving high Cas9 expression in sperm cells would include the DUO1 homologs in wheat, rice, barley, tomato, sunflower, or other monocots or dicots. Other suitable promoters for this concept are shown in Table 8 below. These promoters, or their homologs in crop species—might be very useful for this concept. The principal at work is that gamete cell expression of the editing machinery can increase the rate or efficiency of this invention because it means that there will be abundant editing protein or RNA present or delivered to the embryo during fertilization so that editing can happen rapidly.

TABLE 8
Promoters List: promoters one can use in a transgene to drive high
sperm cell expression of editing machinery to boost the efficiency
of simultaneous editing and doubled-haploid induction (“SEDHI”).

| Gene | Gene | Maize | Rice |
|---|---|---|---|
| Name | ID | Ortholog | Ortholog |
| DUO1 | At3G60460 | GRMZM2G105137, | LOC_Os04g46384 |
| | | GRMZM2G046443 | |
| MGH3 | At1G19890 | NA | NA |
| GEX1 | At5G55490 | GRMZM2G388045 | LOC_Os09g27040, |
| | | | LOC_Os07g47194 |
| GEX2 | At5G49150 | GRMZM2G036832 | LOC_Os09g25650 |
| GEX3 | At5G16020 | GRMZM2G458159 | LOC_Os01g42060 |
| HAP2/GSC1 | At4G11720 | GRMZM2G412911 | LOC_Os05g18730 |
| CycB1 | At4G37490 | NA | NA |
| DAZ1 | At2G17180 | GRMZM2G132057 | NA |
| DAZ2 | At4G35280 | NA | LOC_Os02g19180 |
| DAZ3 | At4G35700 | NA | NA |
| PCR11 | At1G68610 | NA | NA |
| DAN1 | At3G04620 | NA | NA |
| TIP1 | AT3G47440 | NA | LOC_Os04g46490 |
| MKKK20 | AT3G50310 | NA | NA |
| DAF1 | At3G62230 | NA | NA |
| DAW1 | At4G35560 | GRMZM2G176647 | NA |
| DAU2/DMP9 | At5G39650 | NA | NA |

### VII. Simultaneous Haploid Induction and Editing in Dicots Via Wide Cross or Via Crosses to CENH3-Altered Lines or Other Haploid Inducing Lines.

In vivo haploid induction can also be achieved using interspecific or intergeneric wide crosses on dicot plant species, for example, in cotton (Turcotte et al. 1969, Semigametic production of haploids in pima cotton. Crop Sci. 9:653-655) and tobacco (Burke et al, 1979, Maternal haploids of Nicotiana tabacum L. Science 206:585; Wernsman et al. 1989, Androgenetic vs. gynogenetic doubled haploids of tobacco. Crop Sci. 29:1151-1155). Haploid Arabidopsis plants can be obtained by crossing with pollen from mutant CENH3 plant, or by crossing said plants as females to wild type pollen (Ravi and Chan, 2010, Haploid plants produced by centromere-mediated genome elimination. Nature 464: 615-618). Other candidate genes which may be modified to generate a haploid inducer and SEDHI editing line include KNL2 and CENPC (both of which may operate via centromere-mediated uniparental genome elimination) as well as MSI2 and sunflower PLA2. In this case, the haploid-inducing genome (be it the male or female in the cross) also contains the editing machinery, so that the editing can be achieved during the haploid induction process, with the result being an edited maternal or paternal haploid progeny plant without altered CENH3 or editing transgenes. See, e.g., WO 2017/004375, incorporated herein by reference in its entirety. Transgenic locus expressing editing machinery can be introduced into any dicot crops or their wild relatives of Brassica, tomato, pepper, lettuce, eggplant, soybean, sunflower, sugar beet, cotton, alfalfa, tobacco, and others. The transgenic lines expressing editing machinery are then used as pollen donors, or in the case of CENH3, either pollen donors or acceptors, in interspecific or intergeneric wide crosses for haploid induction and simultaneous genome editing. For example, N. africana transgenic CRISPR-Cas9 lines expressing sgRNA targeting tobacco gibberellin 20-oxidase are created through Agrobacterium-mediated transformation and used to pollinate emasculated tobacco flowers to induce haploid plants with their genome edited at the gibberellin 20-oxidase locus. Preferably, an easily transformable line with large number of pollen is used as pollen donor for haploid induction and to provide the editing machinery transiently. The recipient plant for haploid production has flowers that are easy to emasculate or is male sterile. More preferably, a color or other visual marker is present in the induction line or is included in the editing locus to easily differentiate haploid embryos or plants from diploids resulted from normal zygote development.

We exemplified this by utilizing an Arabidopsis haploid inducer line in the Columbia ecotype, and transforming it with a construct encoding expression of Cas9 and a single guide RNA targeting the GLABROUS1 gene (GL1) which, when knocked out, gives a trichome-less phenotype. We crossed the T0s as females by Landsberg Erecta (Ler) ecotype pollen, and recovered gl1 edited haploid progeny.

The haploid inducer materials were obtained from the Comai lab at UC Davis. These materials are typically utilized as paternal haploid inducer lines (causing androgenesis, when crossed as females to wild-type males) but can also act as maternal haploid inducers (causing gynogenesis, when crossed as males to wild-type females). These lines have been altered to become haploid inducers by replacing the native CENH3 gene with a Zea mays CENH3 transgene as reported in (Maheshwari, et al, 2017, Centromere location in Arabidopsis is unaltered by extreme divergence in CENH3 protein sequence. Genome Research 27(3)).

In particular, both copies of the native AtCENH3 gene was knocked out and complemented with the stably inserted ZmCENH3 transgene, which did not impact normal plant development, and did not produce haploids upon self-pollination, but did produce about 10% haploids upon outcross. This is a modification to the original concept of CENH3-tailswap described in detail in (Ravi and Chan, 2010, Haploid plants produced by centromere-mediated genome elimination. Nature 464: 615-618) and many subsequent publications.

After we obtained the CENH3* lines from UC Davis, we grew them up, confirmed that they had the ZmCENH3 transgene and were homozygous “null” for the native AtCENH3 gene. We did this by designing a taqman qPCR assay for ZmCENH3 (assay #2298) and by using PCR and gel electrophoresis to test 183 seedlings for the zygosity of the AtCENH3 genotype by running PCR using the Xbal forward and reverse primers (SEQ NO TKX and TKY) and Reddy mix at 60° C. annealing temperature and cutting with the Xbal restriction enzyme overnight at 37° C. The wildtype allele would be cut by this enzyme and produce two bands (189 bp, 25 bp) while the mutant would remain at 215 bp. These tests showed that all of the seed that UC Davis sent were homozygous for the mutant allele Atcenh3-1, and that there were multiple copies of the ZmCENH3 transgene present.

Confident that these acquired seeds were indeed haploid inducers, we kept 100 plants and initiated floral dip transformation with binary vector 24075 (SEQ ID NO: 98) containing a sgRNA cassette targeting the Arabidopsis (GL1) gene (AT3G27920) at two target sites. The target sequences are 5′-GGAAAAGTTGTAGACTGAGA-3′, and 5′-GCAGTGATGAACAATGACGG-3′ (complementary strand). The disruption of the GL1 gene produces visible phenotypes of partially or completely glabrous plants (glabrous plants lack trichomes). The Cas9 gene (cCas9-05) in this vector was driven by the Arabidopsis thaliana elongation factor promoter. The vector also contains two selectable marker cassettes conferring Kan resistance and AmCyan florescence driven by the CMP-02 promoter and Glycine max UBI-01 promoter respectively. The vector was moved into the Agrobacterium strain EHA101 and then floral dip transformed into the haploid inducer Arabidopsis plants.

The transformation protocol was as follows: In the morning we spread 24075 EHA101 RecA Agrobacterium obtained from plates to YPSpec100Kan50 plates. We cultured these in 28° C. dark for 24 hours. We prepared infiltration medium (4 L): ½ XMS salts (8.66 g), 1× Gamborg's B5 vitamins (4 ml), 5% (W/V) sucrose (200 g), 0.044 μM BAP (12.5 mg-12.5mlDMSO) 40 μL, followed by filter sterilization. We then added 250 μl 40 mg/ml AS (20 mg/L) and 25 μl SIlwet L-77 (50 μl/L) to 500 ml Infiltration media. Using a loop to collect the Agrobacterium and put in 50 ml tube with ˜10 ml o the filter sterilization, we suspended the Agrobacterium until it produced 1 L with an optical density 600 of 0.54. We dipped the inflorescence shoot in to the suspension medium for 20-30 seconds and used the lid to cover the tray. We repeated this for a second time with another suspension of OD600 of 0.552.

About 4 weeks after transformation, approximately 100,000 self-pollinated seeds were harvested and incubated at 4° C. for two days vernalization, and then the seeds were sterilized by soaking in 70% ethanol for 1 minute and then soaking in 50% (V/V) bleach with 0.05% (v/v) Triton X-100 for a further 10 minutes, then washing the seeds in four changes of sterile water. The seeds were then placed on kanamycin (50 μg/ml) plates for germination-screening/selection in a plant tissue culture room (23° C. day, 24° C. night, 16 hours lighting). 38 positive transformants were identified because they were resistant to the kanamycin selection, and they were grown into seedlings before being transferred onto soil and sampled to test for the presence of the Cas9 T-DNA (assay #3049) as well as the status of the two guide RNA cut sites (assays #3321 and #3322). 10 single copy and 15 2-copy events were identified that had both alleles of GL1 mutated and that had a trichomeless phenotype. These plants were prioritized because they had shown evidence of Cas9 activity (by virtue of the mutated GL1 and the glabrous phenotype), they had the Cas9 transgene and they had the ZmCENH3 transgene by qPCR assay. These plants were induced to flower for a long period of time by keeping them in the following growth conditions: 16 hours light, 23° C. Day 20° C. night temperature, not >60% relative humidity.

At the same time as these haploid inducer plants that were transformed with the Cas9 construct were being identified, we were sowing and growing a population of Landsberg Erecta (Ler) seed obtained from the Arabidopsis Biological Resource Center at Ohio State University (line #CS20). These are wild type seed and the sequence of the GL1 guide RNA target sites in CS20 match that of the guide RNA in our construct. We allowed both populations to flower and made about 2000 controlled crosses, using the wild-type Ler plants as the male pollen-donor, crossing onto the approximately 25 haploid inducers with the Cas9 construct, which was used as the female. We made up to 100 crosses per female, marking the crossed flowers with a black marker and removing flowers that we did not cross so as to limit the potential of harvesting self-pollinated siliques. In most cases, we emasculated the female flowers prior to pollination by removing the anthers with forceps, again to avoid contamination with self-pollinated seed, but in some cases this was not necessary because the anthers were young or mal-developed.

About 15 days we harvested the siliques which had developed a light brown color. Then we opened the siliques and planted the seeds in the soil. Then put them in the 6° C. (day and light), 8 hours day length, 200 umal/m²s lighting, 60% relative humidity growth chamber for 4 days. Then we transferred them to 16 hours light, 23° C. Day, 20° C. night temperature, not >60% humidity growth chamber for 7-10 days. We observed a high frequency of aborted seed in almost all of the siliques, averaging about 40-50% of the total seeds. This number of aborted embryos is very consistent with the performance of this haploid inducer material in published reports. Without wishing to be constrained by this theory, it has been speculated that the aborted seed is most likely caused by partial or complete genome elimination in the endosperm leading to endosperm imbalance and failure. This is a natural phenomenon in CENH3-type haploid inducer lines during outcross and is likely not connected with the presence of the Cas9 transgene. These aborted embryos do not germinate. Because of the steady and reliable rate of embryo abortion in every outcrossed silique, we ended up using the absence of that phenotype to screen away siliques that were accidental self-pollinations. That way we germinated siliques that had been outcrossed.

In total we recovered approximately 2000 germinated progeny, the majority of which were outcrossed. We identified the edited haploids via a combination of qPCR marker assays and/or phenotypic screening. The markers that we used to detect the edited haploids were as follows.

First, we looked for a “0” score for the ZmCENH3 assay. This indicates that the plant is a haploid because the maternal genome has been lost, and so the ZmCENH3 transgene, which is present in multiple copies of the mother haploid inducer plant, has also been lost. The diploids, in contrast, will be hybrids between the maternal and paternal genome, and will have a “1” or “2” or higher Taqman score for this assay, depending on the copy number of the mother plant. The key is that all diploids will show evidence of this transgene, but paternal haploids, having only the Ler genome, will not and will thus be a “0.”

Second, we looked for a “0” score for the Cas9 assay, which indicates that it is non-transgenic. This can also be seen visually by using a fluorescent light and looking for the CFP fluorescent marker.

Third, we looked for a “0” score for one of the GL1 target site assays, which indicates that the plant has been edited. The diploid plants might show a “0,” “1” or “2” for those assays, but the haploids either showed a “2” or a “0.” The first of the two GL1 guide RNAs apparently had a much higher editing efficiency than the second, because assay 3321 had a high preponderance of “0”s and “1”s in the haploid inducer T0s, but 3322 had mostly “2”s.

Using these assays, we were able to identify unedited haploids (which were “0” for ZmCENH3 and Cas9, but had “2” scores for both GL1 target sites) and also edited haploids (which had a “0” for the ZmCENH3, Cas9 and GL1 (3321) assays). We were also able to identify diploid hybrids that had Cas9 (and often were edited at the GL1 sites) and diploid hybrids that did not have Cas9 (and often had one copy of GL1 edited (from the maternal parent) but not the other, and thus had a score of “1” for the GL1 assay. We were also able to identify several putative edited haploids because they had a score of “0” for the target site assay (3321), the ZmCENH3 (2298) and the Cas9 (3049). See Table 9 below for an example of progeny Taqman data from parent USR01424136 containing three putative edited haploids (plant 254 in well F2, plant 260 in well D3, and plant 261 in plant E3).

TABLE 9
Progeny analysis from parent USR01424136.

| | | AtGL1-1 cut | | AtGL1-2 cut | | ZmCENH3 | | Cas9 | | | |
|---|---|---|---|---|---|---|---|---|---|---|---|
| | | site 3321 | | site 3322 | | 2298 | | 3049 | | | |
| PLATE 1045 | | Raw | | Raw | | Raw | | Raw | | | |
| HI parent was single copy Cas9 | | Copy | Copy# | Copy | Copy# | Copy | Copy# | Copy | Copy# | Putative | Putative |
| Well | Plant ID | # | level | # | level | # | level | # | level | Haploid | Edited |
| E2 | USR01424136 X | 0.06 | 0 | 0.87 | 1 | 4.30 | >2 | 2.93 | >2 | | x |
| | Ler-253 | | | | | | | | | | |
| F2 | USR01424136 X | 0.00 | 0 | 0.32 | 0 or 1 | 0.00 | 0 | 0.00 | 0 | x | x |
| | Ler-254 | | | | | | | | | | |
| G2 | USR01424136 X | 1.32 | 1 or 2 | 2.06 | 2 | 3.16 | >2 | 0.00 | 0 | | |
| | Ler-255 | | | | | | | | | | |
| H2 | USR01424136 X | 0.02 | 0 | 0.99 | 1 | 2.51 | >2 | 2.99 | >2 | | x |
| | Ler-256 | | | | | | | | | | |
| A3 | USR01424136 X | 0.04 | 0 | 0.87 | 1 | 2.40 | 2 | 2.84 | >2 | | x |
| | Ler-257 | | | | | | | | | | |
| B3 | USR01424136 X | 0.03 | 0 | 1.64 | 2 | 2.99 | >2 | 3.17 | >2 | | x |
| | Ler-258 | | | | | | | | | | |
| C3 | USR01424136 X | 0.03 | 0 | 1.21 | 1 | 5.28 | >2 | 5.28 | >2 | | x |
| | Ler-259 | | | | | | | | | | |
| D3 | USR01424136 X | 0.06 | 0 | 2.01 | 2 | 0.00 | 0 | 0.00 | 0 | x | x |
| | Ler-260 | | | | | | | | | | |
| E3 | USR01424136 X | 0.00 | 0 | 2.01 | 2 | 0.01 | 0 | 0.00 | 0 | x | x |
| | Ler-261 | | | | | | | | | | |
| F3 | USR01424136 X | 2.04 | 2 | 2.10 | 2 | 0.00 | 0 | 0.00 | 0 | x | |
| | Ler-262 | | | | | | | | | | |
| G3 | USR01424136 X | 1.36 | 1 or 2 | 1.25 | 1 | 0.00 | 0 | 0.00 | 0 | x | |
| | Ler-263 | | | | | | | | | | |
| H3 | USR01424136 X | 1.75 | 2 | 1.71 | 2 | 0.00 | 0 | 0.00 | 0 | x | |
| | Ler-264 | | | | | | | | | | |
| A4 | USR01424136 X | 0.00 | 0 | 1.67 | 2 | 3.06 | >2 | 3.16 | >2 | | x |
| | Ler-265 | | | | | | | | | | |
| B4 | USR01424136 X | 1.66 | 2 | 2.32 | 2 | 0.00 | 0 | 0.00 | 0 | x | |
| | Ler-266 | | | | | | | | | | |
| C4 | USR01424136 X | 2.09 | 2 | 1.94 | 2 | 3.99 | >2 | 0.00 | 0 | | |
| | Ler-267 | | | | | | | | | | |
| D4 | USR01424136 X | 1.47 | 1 or 2 | 2.08 | 2 | 6.34 | >2 | 1.51 | 1 or 2 | | |
| | Ler-268 | | | | | | | | | | |
| E4 | USR01424136 X | 1.95 | 2 | 1.76 | 2 | 3.19 | >2 | 0.00 | 0 | | |
| | Ler-269 | | | | | | | | | | |
| F4 | USR01424136 X | 1.92 | 2 | 2.17 | 2 | 4.28 | >2 | 0.02 | 0 | | |
| | Ler-270 | | | | | | | | | | |
| G4 | USR01424136 X | 2.02 | 2 | 1.85 | 2 | 4.31 | >2 | 0.00 | 0 | | |
| | Ler-271 | | | | | | | | | | |
| H4 | USR01424136 X | 0.00 | 0 | 1.71 | 2 | 1.65 | 2 | 1.12 | 1 | | x |
| | Ler-272 | | | | | | | | | | |

Simply by germinating seeds and sampling for qPCR Taqman analysis, we were able to identify 8 putative edited haploids. Edited haploids were also identified by phenotypic visual screening, and then confirmed later by Taqman assay. We screened for the edited haploids by looking for trichomeless, or glabrous, plants, which indicated that they did not have any wild-type alleles for the GL1 gene, and by looking for a lack of cyan fluorescent protein (“CFP”) expression in the embryo or seedling root. This indicated that they lacked the Cas9 T-DNA. We observed several of these plants, and submitted them for Taqman assays. For three such plants that we identified phenotypically, we were able to confirm that they were truly edited haploids by the Taqman assays. We were aware of the fact that it is possible that some of these glabrous plants that lack CFP were false positives, either because the CFP was silent or because of self-pollination of the fully-edited mother plant and production of null segregant, fully edited (and thus glabrous) progeny. The Taqman assays were able to detect and screen out these false positives, because they directly tested for the presence of not only the Cas9 transgene, but also the ZmCENH3 allele, which would certainly be present in any self-pollinated contaminating seed. We found several examples of self-pollinated seed that all came from one mother plant. The pollination notes for that mother indicated that there was highly abundant pollen that may have resulted in some self-pollination. We excluded these progeny from the total analysis.

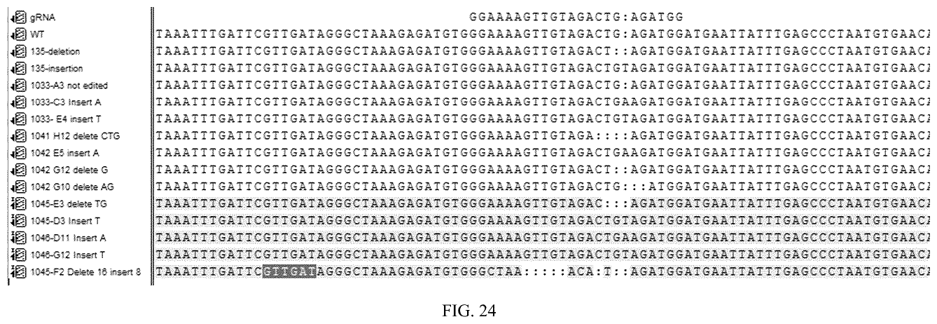

All of the putative edited haploids identified by Taqman assay were sequenced. We used PCR to amplify the edited alleles, and then subcloned and sequenced at least 8 colonies for each putative edited allele. See Table 10 for the sequence changes we found in the edited haploids at the first guide RNA (assay #3321) target site, as well as the Taqman data from the T0 parents. In total, we found 19 putative edited haploids, and we confirmed that the 3321 target sites had mutations in 11 of the 12 edited haploids that we attempted to sequence. Whether the other 7 would also have mutations will be confirmed upon sequencing. See the sequence alignment for these edits in FIG. 24.

TABLE 10
Taqman and sequence data from 19 edited haploids.

| | | | AtGL1-1 cut | | AtGL1-2 cut | | ZmCENH3 | | Cas9 | | | |
|---|---|---|---|---|---|---|---|---|---|---|---|---|
| | | | site 3321 | | site 3322 | | 2298 | | 3049 | | | |
| | | | Raw | Copy | Raw | Copy | Raw | Copy | Raw | Copy | Target | |
| | | | Copy | # | Copy | # | Copy | # | Copy | # | site | PA |
| Plate | Well | Plant ID | # | level | # | level | # | level | # | level | mutation | confirm? |
| 1033 | A3 | USR01424135 X | 0.00 | 0 | 1.67 | 2 | 0.04 | 0 | 0.00 | 0 | wild type | Not done |
| | | Ler-425 | | | | | | | | | | |
| 1033 | C3 | USR01424135 X | 0.21 | 0 | 2.43 | >2 | 0.01 | 0 | 0.00 | 0 | insert A | Yes |
| | | Ler-427 | | | | | | | | | | |
| 1033 | E4 | USR01424135 X | 0.08 | 0 | 2.04 | 2 | 0.00 | 0 | 0.00 | 0 | insert T | Yes |
| | | Ler-437 | | | | | | | | | | |
| 1042 | E5 | USR01424136 X | 0.16 | 0 | 2.95 | >2 | 0.00 | 0 | 0.00 | 0 | insert A | Not done |
| | | Ler-25 | | | | | | | | | | |
| 1042 | G10 | USR01424136 X | 0.00 | 0 | 2.19 | 2 | 0.00 | 0 | 0.00 | 0 | delete AG | Not done |
| | | Ler-67 | | | | | | | | | | |
| 1042 | G12 | USR01424136 X | 0.00 | 0 | 1.86 | 2 | 0.00 | 0 | 0.00 | 0 | delete G | Not done |
| | | Ler-83 | | | | | | | | | | |
| 1043 | B11 | USR01424136 X | 0.16 | 0 | 1.59 | 1 or 2 | 0.01 | 0 | 0.00 | 0 | Not done | Not done |
| | | Ler-154 | | | | | | | | | | |
| 1045 | F2 | USR01424136 X | 0.00 | 0 | 0.32 | 0 or 1 | 0.00 | 0 | 0.00 | 0 | delete 8nt* | Not done |
| | | Ler-254 | | | | | | | | | | |
| 1045 | D3 | USR01424136 X | 0.06 | 0 | 2.01 | 2 | 0.00 | 0 | 0.00 | 0 | insert T | Not done |
| | | Ler-260 | | | | | | | | | | |
| 1045 | E3 | USR01424136 X | 0.00 | 0 | 2.01 | 2 | 0.01 | 0 | 0.00 | 0 | delete TG | Not done |
| | | Ler-261 | | | | | | | | | | |
| 1046 | D11 | USR01431609 X | 0.09 | 0 | 1.59 | 1 or 2 | 0.02 | 0 | 0.01 | 0 | insert A | Not done |
| | | Ler-111 | | | | | | | | | | |
| 1046 | G12 | USR01431609 X | 0.02 | 0 | 1.62 | 2 | 0.00 | 0 | 0.00 | 0 | insert T | Not done |
| | | Ler-122 | | | | | | | | | | |
| 1046 | H12 | USR01431609 X | 0.00 | 0 | 0.00 | 0 | 0.00 | 0 | 0.00 | 0 | delete CTG | Yes |
| | | Ler-123 | | | | | | | | | | |
| 0583 | D12 | USR01431603 X | 0.00 | 0 | 1.50 | 1 or 2 | 0.00 | 0 | 0.00 | 0 | Not done | Not done |
| | | Ler-80 | | | | | | | | | | |
| 0584 | A9 | USR01431603 X | 0.00 | 0 | 1.87 | 2 | 0.00 | 0 | 0.00 | 0 | Not done | Not done |
| | | Ler-137 | | | | | | | | | | |
| 0584 | C11 | USR01431603 X | 0.05 | 0 | 2.06 | 2 | 0.00 | 0 | 0.17 | 0 | Not done | Not done |
| | | Ler-155 | | | | | | | | | | |
| 0584 | G11 | USR01431603 X | 0.09 | 0 | 2.15 | 2 | 0.00 | 0 | 0.00 | 0 | Not done | Not done |
| | | Ler-159 | | | | | | | | | | |
| 0584 | C12 | USR01431603 X | 0.00 | 0 | 1.35 | 1 or 2 | 0.00 | 0 | 0.11 | 0 | Not done | Not done |
| | | Ler-163 | | | | | | | | | | |
| 0584 | F12 | USR01431603 X | 0.00 | 0 | 1.65 | 2 | 0.00 | 0 | 0.00 | 0 | Not done | Not done |
| | | Ler-166 | | | | | | | | | | |
| 0585 | H7 | USR01431603 X | 0.06 | 0 | 2.05 | 2 | 0.00 | 0 | 0.01 | 0 | Not done | Not done |
| | | Ler-212 | | | | | | | | | ΔG, +T | |
| Female | | USR01424135 | 0.03 | 0 | 1.42 | 1 or 2 | 4.46 | >2 | 2.98 | >2 | chimera | Diploid |
| Parent | | USR01424136 | 0.03 | 0 | 1.13 | 1 | 3.59 | >2 | 2.76 | >2 | Not done | Diploid |
| TO | | USR01431603 | 0.14 | 0 | 1.25 | 1 | 2.48 | >2 | 3.42 | >2 | Not done | Diploid |
| Plants | | USR01431609 | 0.18 | 0 | 1.1 | 1 | 4.75 | >2 | 5.57 | >2 | Not done | Diploid |

*delete 16 nt insert CTAAACAT

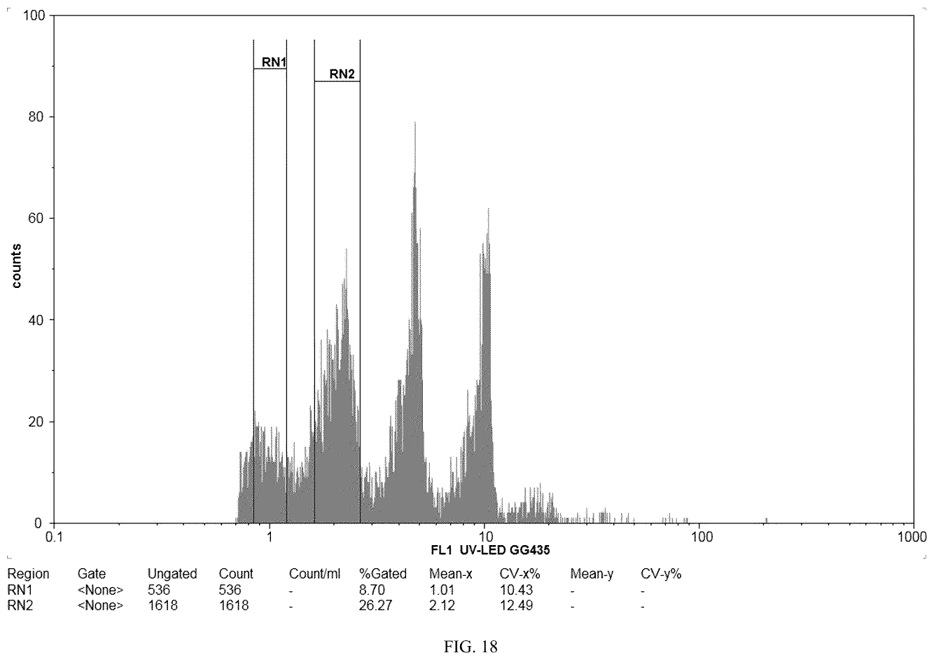

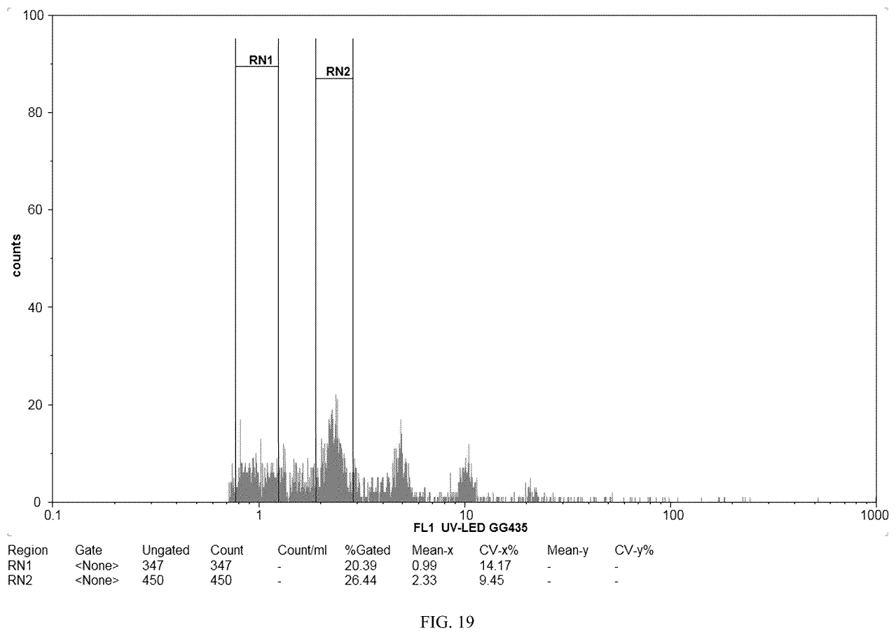

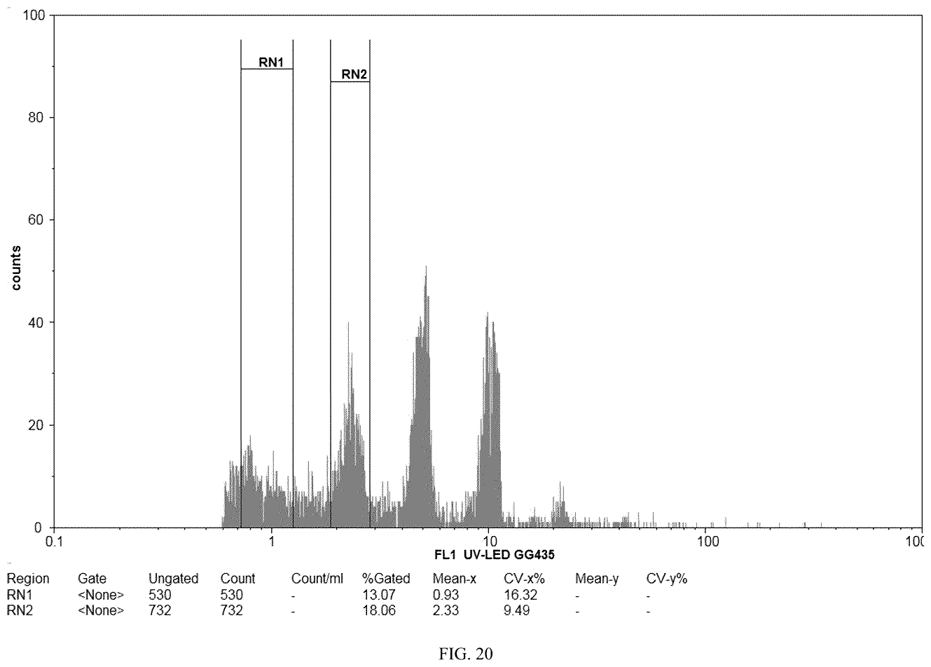

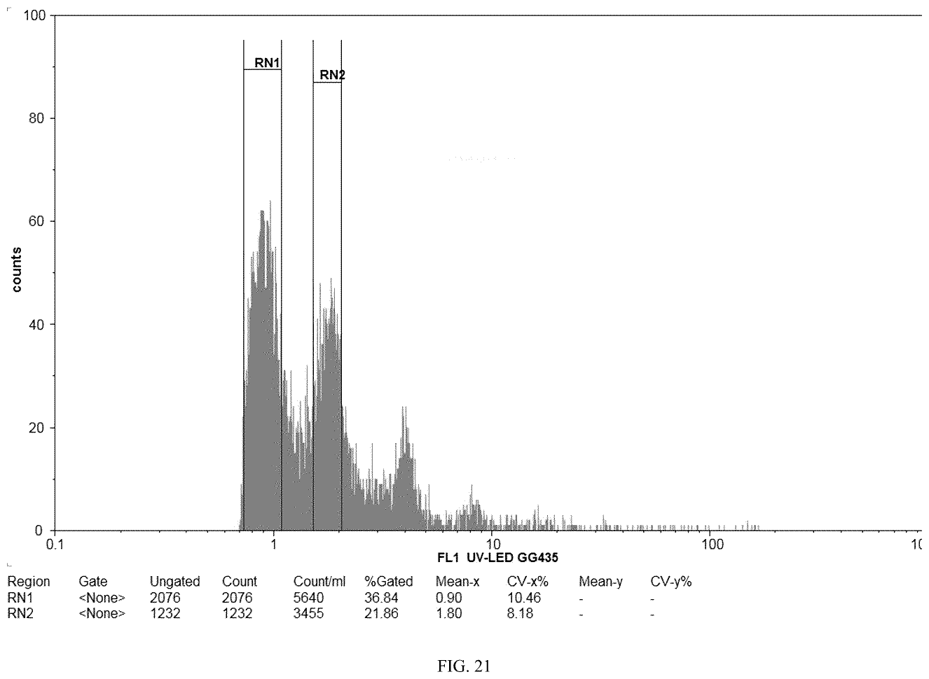

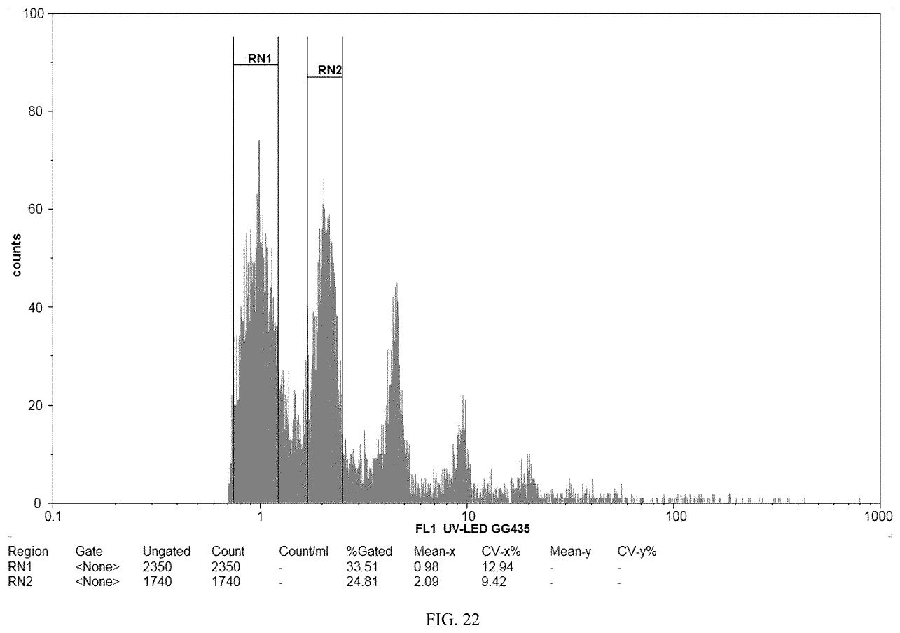

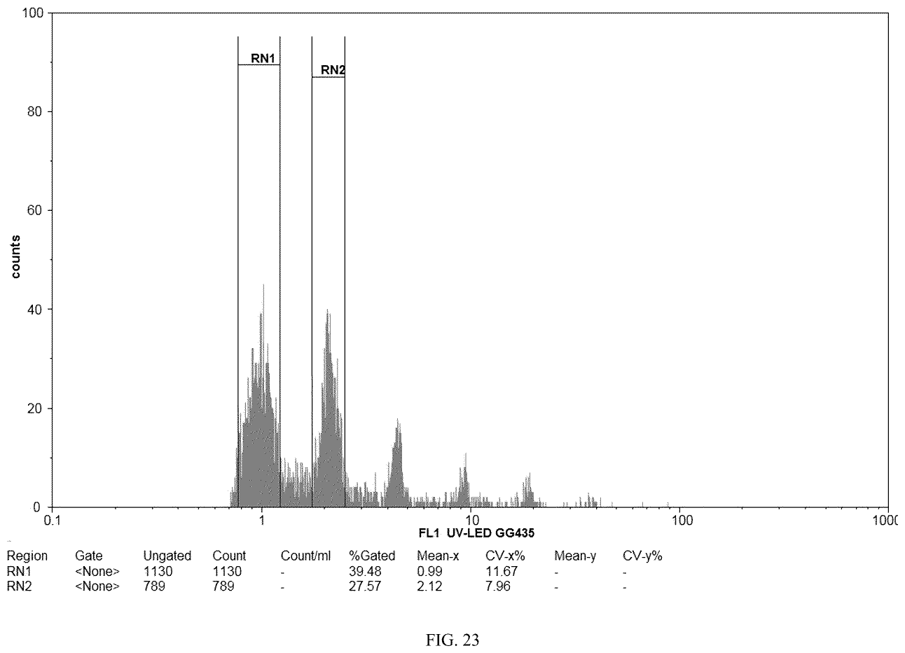

We further ran leaf samples from three edited haploid plants through ploidy analysis, along with three diploid controls (tissue sampled from the maternal parent plants), which showed that they were true haploids (FIGS. 18-23). This served to reconfirm their status as edited haploids.

In three parental lines where we were confident that there was no self-pollination contamination, we did not do any phenotypic pre-screening, but instead sampled all germinated progeny for Taqman analysis (Table 11). The three female parents for these progeny were USR01431603, USR01431609, and USR01431604. We found a haploid induction rate of about 9.7% calculated by dividing the number of progeny that lack the ZmCENH3 and Cas9 transgenes (59) by the total number of progeny sampled (605). Of the 59 haploids we found that 10 were edited. That means 16.9% of haploids, on average, were edited by the maternal Cas9, prior to elimination of the maternal genome. Without wishing to be constrained by this final number, this means that, using this system, as a percentage of total progeny, 9.7%*16.9%=1.64% of all germinated progeny were edited haploids.

TABLE 11
Haploid induction rate and editing rate data from three sets of progeny,
each derived from a different SEDHI inducer female parent
crossed by Landsberg erecta pollen.

| | Parent | Parent | | | | | Edited |
|---|---|---|---|---|---|---|---|
| | plant | plant | Total | Haploid | Haploid | Edited | Haploid |
| ID | Cas9-05 | cNpt2-10 | [symbol] | number | rate | Haploid | rate |
| USR01431603 X | >2 | >2 | 230 | 36 | 15.65 | 7 | 19.44 |
| Landsberg erecta | | | | | | | |
| USR01431603X | >2 | >2 | 123 | 14 | 12.38 | 3 | 21.43 |
| Landsberg erecta | | | | | | | |
| USR01431604 X | 2 | 1 | 252 | 9 | 3.57 | 0 | 0.00 |
| Landsberg erecta | | | | | | | |

[symbol]

indicates data missing or illegible when filed

The rate of CENH3* type haploid editing or other paternal haploid editing (using a maternal haploid inducer line) might be increased through the use of a promoter that drives the expression of Cas9 and/or the guide RNA to a higher level in the egg cell before fertilization and/or in the zygote cell during or after fertilization. An example of such a promoter would the promoter for EA1 (EGG APPARATUS1) (GRMZM2G456746), although there are many other examples. One could also express the Cas9 in the context of an egg apparatus-specific enhancer (EASE), which is a 77-bp sequence that stimulates expression of adjoining genes in the egg cell or the very early zygote (see, e.g., Yang, et al. An Egg Apparatus-Specific Enhancer of Arabidopsis, Identified by Enhancer Detection, PLANT PHYSIOLOGY November 2005, 139 (3) 1421-1432; DOI: https://doi.org/10.1104/pp.105.068262).

### VIII. Simultaneous Haploid Induction and Editing by Directly Modifying a Target Base in Genomic DNA Sequence.

Targeted mutagenesis of DNA sequence can also be achieved through direct conversion of one DNA base to another without requiring double stranded breaks (DSBs). For example, cytidine deaminase APOBEC1, adenine deaminase, and other enhancing components like Uracil DNA glycosylase (UDG) can be fused to Cas9 (A840H) nickase or nuclease-inactivated dead Cas9 (dCa9) to direct editing of DNA sequence without introducing double strand DNA breaks (Komor et al. 2016. Programmable editing of a target base in genomic DNA without double-stranded DNA cleavage. Nature doi:10.1038/nature17946; Gaudelli et al. 2017. Programmable base editing of A:T to G:C in genomic DNA without DNA cleavage. Nature doi:10.1038/nature24644; Komor et al. 2017. Improved base excision repair inhibition and bacteriophage Mu Gam protein yields C:G-to-T:A base editors with higher efficiency and product purity. Science Advances, Vol. 3, no. 8, eaao4774, DOI: 10.1126/sciadv.aao4774). This kind of base editor machinery can also be delivered through haploid induction line to induce base editing in target sequences directly in other varieties. For example, a guide RNA sequence, xZmVLHP-03 (5′-AGGCGTCGAGCAGCGAGGTG-3′, SEQ ID NO: 28) is designed to target the cytidine deaminase base editor system to convert ZmVLHP gene exon 2 genomic sequence 5′-AGGCGTCGAGCAGCGAGGTG-3′ (SEQ ID NO: 28) into 5′-AGGCGTTGAGCAGCGAGGTG-3′ (SEQ ID NO: 29), thus changing the arginine codon CGA into a stop codon (TGA) in the coding sequence and causing premature termination of the protein sequence and functional gene knockout. The C to T mutation is underlined. Similarly, chimeric nCas9- or dCas9-adenine deaminase base editing system can be used to mutate the coding region, splicing junction or promoter sequence of ZmVLHP or other genes to generate variants that have altered gene activity. Both cytidine and adenine deaminase are particularly useful for altering transcript splicing site since canonical splicing junction has 5′- . . . AG/GT . . . 3′ sequence (or 5′- . . . AC/CT . . . 3′ in the opposite strand).

IX. Simultaneous Haploid Induction and Editing by Allele Replacement with DNA Template

Not only can in vivo haploid induction system be used to introduce protein, RNA or DNA for cleavage or conversion of target sequence, it can also be used to deliver DNA template for homology-dependent repair for precise sequence replacement in the target region in the form of transgenic DNA. The template DNA can be inserted into the inducer line genome carrying genome editing machinery such as CRISPR-Cas9 system, either in the same transgenic locus or different locus. When both Cas9-sgRNA and template DNA are present in the induced haploid embryos, cleavage of the target sequence will result in repair of the chromosomal break with the homologous transgenic DNA sequence as template. For example, for creating E149L mutation in ZmPYL-D gene (GRMZM2G048733_P02) (see WO16033230, incorporated herein by reference), DNA fragment containing donor sequence (5′-CCTTGGTGTTGCCGTCGGGGACGTCGACGACGAATGACAGGATGACGAGCGTCC CTGGCCGGCCGTCGATGACCT-3′, SEQ ID NO: 30) is used as repair donor. It should be noted that additional homology sequences can be added to flank this core repair donor sequence. One or more copies of this repair donor sequence are inserted into Cas9-sgRNA expression vector 23136 (SEQ ID NO: 31) which expresses guide RNA 5′-GTCGGGGACGTCGACGACGA-3′ (SEQ ID NO: 32) to form allele modification vector pBSC23136-AMD. It should be noted that the potential PAM site has been removed from the donor DNA sequences so that the integrated donor sequence will not be cleaved by the Cas9-sgRNA complex expressed from pBSC23136-AMD. pBSC23136-AMD is transformed into haploid inducer line NP2222-HI to generate transgenic editing line. Transgenic editing-haploid induction lines are selfed to produce progeny lines homozygous editing loci. These homozygous lines are used to pollinate target elite maize inbred lines to induce haploid formation and also introduce modified alleles by expressed Cas9-sgRNA using donor DNA present transiently before pollen donor chromosomes are eliminated.

### X. Inducing Haploids and Simultaneous Gene Editing in Rice

A HI-rice line is obtained. For example, the rice MATL ortholog, Os03g27610 (SEQ ID NO: 33, is mutated to create a new rice HI line. This line is transformed with a vector comprising a site-directed mutagenesis system for editing the rice genome, for example the CRISPR/Cas9 system.

The rice HI line is crossed with a different rice line, preferably an elite line, to produce at least one progeny haploid embryo. During the cross to produce at least one progeny haploid embryo, the HI parent rice plant also causes the genome editing machinery, e.g., Cas9 plus a guide RNA, to be delivered to the embryo. At that point, the editing machinery operates to edit the genome of the haploid embryo, and thus an edited, haploid progeny plant is obtained.

### XI. Taqman Assays and Conditions.

Several assays are mentioned by number or by target name. Provided below is a table of assays mentioned above and the sequences of the relevant primers and probes. Conditions for PCR are standard for all assays and are as follows: Denature at 98° C. for 2 minutes; followed by 35 cycles of (i) denature at 98° C. for 30 seconds, (ii) anneal at 60° C. for 30 seconds, (iii) extension at 72° C. for 1 minute; followed by final extension at 72° C. for 10 minutes with a hold at 4° C. until ready. Assays are carried out at these conditions unless otherwise noted below.

TABLE 11
Assay primers and probes.

| Target | Cas9-in corn | | SEQ ID |
|---|---|---|---|
| Assay No. | 2540 | Sequence | NO: |
| Forward | FE09340 | TTGTGCTGCTCCACGAACA | 39 |
| Primer | | | |
| Reverse | FE09341 | GCCAGCCACTACGAGAAGCT | 40 |
| Primer | | | |
| Probe | FE09342 | CTGCTTCTGCTCGTTGTCCTCCGG | 41 |
| Target | mat1 | | SEQ ID |
| Assay No. | 2827 | Sequence | NO: |
| Forward | FE10299 | GCGGATGCTGGCACAGC | 42 |
| Primer | | | |
| Reverse | FE10300 | GGCATTGCTTCCTTCTCCG | |
| Primer | | | 43 |
| Probe | FE10301 | CAGGGAGCGAGGTAC | 44 |
| Target | PMI | | SEQ ID |
| Assay No. | 1750 | Sequence | NO: |
| Forward | FE07390 | CTGGTGGCCAACGTGAAGTT | 45 |
| Primer | | | |
| Reverse | FE07391 | GCTTCACGGGCTGGGTC | 46 |
| Primer | | | |
| Probe | FE07392 | AGGCCAAGCCCGCCAACCAG | 47 |
| Target | MATL-WT | | SEQ ID |
| Assay No. | 2826 | Sequence | NO: |
| Forward | FE10297 | GCGGATGCTGGCACAGA | 48 |
| Primer | | | |
| Reverse | FE10298 | GCATTGCTTCCTTCGCCA | 49 |
| Primer | | | |
| Probe | FE10299 | CAGGGAGGTACGAACC | 50 |
| Target | TAV_4A | | SEQ ID |
| Assay No. | 3252 | Sequence | NO: |
| Forward | FE11306 | GCGGCGAAGAAGCGAA | 51 |
| Primer | | | |
| Reverse | FE11307 | GCGGCGTCTCCAGCTTC | 52 |
| Primer | | | |
| Probe | FE11308 | CCAGGAACTGCG | 53 |
| Target | TAV_4B | | SEQ ID |
| Assay No. | 3253 | Sequence | NO: |
| Forward | FE11309 | AAGAAACGCCGGCTGAGT | 54 |
| Primer | | | |
| Reverse | FE11310 | ACCTTGCGGGGCGTT | 55 |
| Primer | | | |
| Probe | FE11308 | CCAGGAACTGCG | 56 |
| Target | TAV_4D | | SEQ ID |
| Assay No. | 3254 | Sequence | NO: |
| Forward | FE11309 | AAGAAACGCCGGCTGAGT | 57 |
| Primer | | | |
| Reverse | FE11311 | CCTTGCGCGGCGTC | 58 |
| Primer | | | |
| Probe | FE11308 | CCAGGAACTGCG | 59 |
| Target | GW2-01 | | SEQ ID |
| Assay No. | 3065 | Sequence | NO: |
| Forward | FE10799 | TGATCCTCGAGGCCAAGCT | 60 |
| Primer | | | |
| Reverse | FE10800 | AGGTCGAGGTCCCCTCCA | 61 |
| Primer | | | |
| Probe | FE10801 | CCTGCTACCCGGGC | 62 |
| Target | GW2-02 | | SEQ ID |
| Assay No. | 3095 | Sequence | NO: |
| Forward | FE10991 | CGCGCCCTGCTACCC | 63 |
| Primer | | | |
| Reverse | FE10992 | GCGCGTGCTTACCAGGA | 64 |
| Primer | | | |
| Probe | FE10993 | TCGAGGAGTGCCC | 65 |
| Target | TaVHLP2-2A | | SEQ ID |
| Assay No. | 3332 | Sequence | NO: |
| Forward | FE11312 | CACCGATGAGCAGGCG | 66 |
| Primer | | | |
| Reverse | FE11313 | AGATACACCTTCCGGCCG | |
| Primer | | | 67 |
| Probe | FE11314 | TTCCTCCCGGAAGC | 68 |
| Target | TaVHLP2-2D | | SEQ ID |
| Assay No. | 3333 | Sequence | NO: |
| Forward | FE11312 | CACCGATGAGCAGGCG | 69 |
| Primer | | | |
| Reverse | FE11313 | AGATACACCTTCCGGCCAGT | 70 |
| Primer | | | |
| Probe | FE11314 | CTCCTCCCGGAAGC | 71 |
| Target | | | SEQ ID |
| Assay No. | 3049 | Sequence | NO: |
| Forward | FE10730 | CAAGTTTCTGGACAAGGAGATTCTC | 72 |
| Primer | | | |
| Reverse | FE10731 | AAGAATTCCCTTCTTAATAGCTGGAGA | 73 |
| Primer | | | |
| Probe | FE10732 | CACGAGCACATTGCTAACCTTGCTGG | 74 |
| Target | TaVHLP2-2B | | SEQ ID |
| Assay No. | 3255 | Sequence | NO: |
| Forward | FE11315 | TCACCGATGAGCAGGCA | 75 |
| Primer | | | |
| Reverse | FE11316 | ATACACCTTCCGGCCAGC | 76 |
| Primer | | | |
| Probe | FE11317 | TTCCTCCCGGAAGC | 77 |
| Target | | | SEQ ID |
| Assay No. | 3321 | Sequence | NO: |
| Forward | FE11540 | GATAGGGCTAAAGAGATGTGGGAA | 78 |
| Primer | | | |
| Reverse | FE11541 | CTTTGTTCACATTAGGGCTCAAATAA | 79 |
| Primer | | | |
| Probe | FE11542 | TAGACTGAGATGGATG | 80 |
| Target | | | SEQ ID |
| Assay No. | 3322 | Sequence | NO: |
| Forward | FE11543 | AAAACCACCGGAGAAGACGA | 81 |
| Primer | | | |
| Reverse | FE11544 | AGGTGTGGCGGCAGTGA | 82 |
| Primer | | | |
| Probe | FE11545 | CACCGTCATTGTTC | 83 |
| | Cas9-in | | |
| Target | Arabidopsis | | SEQ ID |
| Assay No. | 3049 | Sequence | NO: |
| Forward | FE10730 | CAAGTTTCTGGACAAGGAGATTCTC | 84 |
| Primer | | | |
| Reverse | FE10731 | AAGAATTCCCTTCTTAATAGCTGGAGA | 85 |
| Primer | | | |
| Probe | FE10732 | CACGAGCACATTGCTAACCTTGCTGG | 86 |
| Target | ZmCENH3 | | SEQ ID |
| Assay No. | 2298 | Sequence | NO: |
| Forward | FE08737 | GCGACGCCGGAAAGG | 87 |
| Primer | | | |
| Reverse | FE08738 | TGGCGTGGTTTCGTCTTCTTA | 88 |
| Primer | | | |
| Probe | FE08739 | AAGAGCGGCGTCTGGAGGTGACTCA | 89 |
| | GL1 3321 target | | SEQ ID |
| Target | site (PCR) | Sequence | NO: |
| Forward Primer | 3321F | AACCGCATCGTCAGAAAAAC | 90 |
| Reverse Primer | 3321R | TCAACTTAACCGGCCAAATC | 91 |
| Annealing Temp. | 60° C. | | |
| | VLHP2-2A target | | SEQ ID |
| Target | site (PCR) | Sequence | NO: |
| Forward Primer | FE4117 | CATCCCTTCTCTTCCCTCCTG | 92 |
| Reverse Primer | FE4118 | GCCAGTGTGAGTGTGTATGAGCA | 93 |
| Annealing Temp. | 61° C. | | |
| | VLHP2-2B target | | SEQ ID |
| Target | site (PCR) | Sequence | NO: |
| Forward Primer | FE4120 | CATCGTTTTCTCCCCTCCTCA | 94 |
| Reverse Primer | FE4121 | ACTGATATGCACGGCGCCA | 95 |
| Annealing Temp. | 62° C. | | |
| | VLHP2-2D target | | SEQ ID |
| Target | site (PCR) | Sequence | NO: |
| Forward Primer | FE4121 | TGCAGTAGCTTCATTTTCACCG | 96 |
| Reverse Primer | FE4122 | AGGAATTGATATGTACGCCCGT | 97 |
| Annealing Temp. | 61º C. | | |

What is claimed is:

1. A method of editing dicot genomic DNA, comprising:
(a) obtaining a first dicot plant comprising a mutation in a centromeric histone 3 (CENH3) gene and optionally a maize CENH3 tailswap transgene, wherein said first dicot plant expresses a site-directed DNA modification enzyme and optionally at least one guide nucleic acid, wherein the site-directed DNA modification enzyme is a base editor;
(b) obtaining a second dicot plant, wherein the second dicot plant comprises the dicot plant genomic DNA to be edited;
(c) pollinating the first dicot plant with pollen from the second dicot plant; and
(d) selecting at least one haploid progeny produced by the pollination of step (c) wherein the haploid progeny comprises the genome of the second dicot plant but not the first dicot plant, and the genome of the haploid progeny has been modified by the base editor and optional at least one guide nucleic acid delivered by the first dicot plant; wherein the mutation in a CENH3 gene is selected from the group consisting of a loss-of-function mutation, a partial loss-of-function mutation, a restored frameshift mutation, and an in-frame deletion mutation.

2. The method of claim 1, wherein the base editor comprises a site-directed nuclease selected from the group consisting of a Cas9 nuclease, Cpf1 nuclease, dCas9-FokI, dCpf1-FokI, a nickase Cas9 (nCas9), chimeric dCas9 non-FokI nuclease and dCpf1 non-FokI nuclease.

3. The method of claim 1, wherein the base editor comprises a cytidine deaminase fused to a Cas polypeptide.

4. The method of claim 1, wherein the cytidine deaminase is an APOBEC deaminase.

5. The method of claim 1, wherein the base editor comprises an adenine deaminase fused to a Cas polypeptide.

6. The method of claim 1, wherein the base editor comprises a uracil DNA glycosylase fused to a Cas polypeptide.

7. The method of claim 1, wherein an edited haploid progeny selected in (d) is treated with a chromosome doubling agent, thereby creating an edited doubled haploid progeny.

8. The method of claim 7, wherein the chromosome doubling agent is colchicine, pronamide, dithipyr, or trifluralin.

9. The method of claim 1, wherein the edited haploid progeny undergoes spontaneous chromosome doubling, thereby creating an edited doubled haploid progeny.

10. The method of claim 1, wherein the guide nucleic acid is a guide RNA.

11. The method of claim 10, wherein the guide RNA is an 18-21 nucleotide sequence and is at least 90% identical to a target sequence.

12. The method of claim 8, wherein the target sequence is SEQ ID NO: 103, and wherein the dicot genomic DNA is Arabidopsis genomic DNA.

13. The method of claim 1, wherein the first dicot plant expresses a marker gene.

14. The method of claim 13, wherein the marker gene is selected from the group consisting of GUS, PMI, PAT, GFP, RFP, CFP, B1, C1, R-nj, and anthocyanin pigments.

13. The method of claim 1, wherein the first dicot plant is a transformable dicot plant selected from the group consisting of Arabidopsis, Brassica, soybean, tomato, sunflower, and cotton.

14. The method of claim 1, wherein the partial loss-of-function mutation is a frame-shift mutation near the 3′ terminus of the CENH3 gene.

15. The method of claim 1, wherein the loss-of-function mutation is a knock-out mutation.


SEQUENCE LISTING (117 sequences)

SEQ ID NO: 1 - DNA, 15722 bp, synthetic construct
  misc_feature 1..15722: note = vector 23396
  misc_feature 4..259: note = bNRB-05
  regulatory 304..2100: note = promoter - prSoUbi4-04; regulatory_class = promoter
  gene 2117..6286: note = cCas9-01
  variation 5606..5608: note = mutation - L to V mutation
  variation 5651..5653: note = mutation - I to V mutation
  regulatory 6292..6544: note = terminator - tNOS-05-01; regulatory_class = terminator
  regulatory 6551..6925: note = promoter - prOsU3-01; regulatory_class = promoter
  misc_feature 6927..6946: note = xZmVLHP
  misc_feature 6927..7032: note = rsgRNAZmVLHP-01
  regulatory 7042..9033: note = promoter - prUbi1-04; regulatory_class = promoter
  gene 9050..10228: note = cPMI-09
  regulatory 10251..10503: note = terminator - tNOS-05-01; regulatory_class = terminator
  misc_feature 10547..10676: note = bNLB-03
  gene 10956..11744: note = cSpec-03
  regulatory 11839..11969: note = promoter - prVirG-01; regulatory_class = promoter
  gene 12706..13779: note = cRepA-01
  source 1..15722: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtaccattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggatcgcggccgctcatatggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggacccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaagggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcagcaggaggcgtcgagcagcggttttagagctagaaatagcaagttaaaataaggctagtccgttatcaacttgaaaaagtggcaccgagtcggtgctttttttttcggaccgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccggcagcagccatgcagaagctgatcaacagcgtgcagaactacgcctggggcagcaagaccgccctgaccgagctgtacggcatggagaaccccagcagccagcccatggccgagctgtggatgggcgcccaccccaagagcagcagccgcgtgcagaacgccgccggcgacatcgtgagcctgcgcgacgtgatcgagagcgacaagagcaccctgctgggcgaggccgtggccaagcgcttcggcgagctgcccttcctgttcaaggtgctgtgcgccgcccagcccctgagcatccaggtgcaccccaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgtgagcctgctgcagcccgtggccggcgcccaccccgccatcgcccacttcctgcagcagcccgacgccgagcgcctgagcgagctgttcgccagcctgctgaacatgcagggcgaggagaagagccgcgccctggccatcctgaagagcgccctggacagccagcagggcgagccctggcagaccatccgcctgatcagcgagttctaccccgaggacagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggccaagcccgccaaccagctgctgacccagcccgtgaagcagggcgccgagctggacttccccatccccgtggacgacttcgccttcagcctgcacgacctgagcgacaaggagaccaccatcagccagcagagcgccgccatcctgttctgcgtggagggcgacgccaccctgtggaagggcagccagcagctgcagctgaagcccggcgagagcgccttcatcgccgccaacgagagccccgtgaccgtgaagggccacggccgcctggcccgcgtgtacaacaagctgtgataggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccgcaattgaagtttgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 2 - DNA, 20 bp, synthetic construct
  misc_feature 1..20: note = gRNA sequence for editing VLHP1
  source 1..20: mol_type = other DNA
gcaggaggcgtcgagcagcg

SEQ ID NO: 3 - DNA, 15722 bp, synthetic construct
  misc_feature 1..15722: note = vector 23399
  misc_feature 4..259: note = bNRB-05
  regulatory 304..2100: note = promoter - prSoUbi4-04; regulatory_class = promoter
  gene 2117..6286: note = cCas9-01
  variation 5606..5608: note = mutation - L to V mutation
  variation 5651..5653: note = mutation - I to V mutation
  regulatory 6292..6544: note = terminator - tNOS-05-01; regulatory_class = terminator
  regulatory 6551..6925: note = promoter - prOsU3-01; regulatory_class = promoter
  misc_feature 6927..6946: note = xZmGW2
  misc_feature 6927..7031: note = rsgRNAZmGW2-02
  regulatory 7042..9033: note = promoter - prUbi1-04; regulatory_class = promoter
  gene 9050..10228: note = cPMI-09
  regulatory 10251..10503: note = terminator - tNOS-05-01; regulatory_class = terminator
  misc_feature 10547..10676: note = bNLB-03
  gene 10956..11744: note = cSpec-03
  regulatory 11839..11969: note = promoter - prVirG-01; regulatory_class = promoter
  gene 12706..13779: note = cRepA-01
  misc_feature 13822..14226: note = oVS1-02
  misc_feature 14904..15710: note = oCOLE-06
  source 1..15722: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtaccattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggatcgcggccgctcatatggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggacccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaagggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcaaagctcgcgccctgctacccgttttagagctagaaatagcaagttaaaataaggctagtccgttatcaacttgaaaaagtggcaccgagtcggtgctttttttttcggaccgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccggcagcagccatgcagaagctgatcaacagcgtgcagaactacgcctggggcagcaagaccgccctgaccgagctgtacggcatggagaaccccagcagccagcccatggccgagctgtggatgggcgcccaccccaagagcagcagccgcgtgcagaacgccgccggcgacatcgtgagcctgcgcgacgtgatcgagagcgacaagagcaccctgctgggcgaggccgtggccaagcgcttcggcgagctgcccttcctgttcaaggtgctgtgcgccgcccagcccctgagcatccaggtgcaccccaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgtgagcctgctgcagcccgtggccggcgcccaccccgccatcgcccacttcctgcagcagcccgacgccgagcgcctgagcgagctgttcgccagcctgctgaacatgcagggcgaggagaagagccgcgccctggccatcctgaagagcgccctggacagccagcagggcgagccctggcagaccatccgcctgatcagcgagttctaccccgaggacagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggccaagcccgccaaccagctgctgacccagcccgtgaagcagggcgccgagctggacttccccatccccgtggacgacttcgccttcagcctgcacgacctgagcgacaaggagaccaccatcagccagcagagcgccgccatcctgttctgcgtggagggcgacgccaccctgtggaagggcagccagcagctgcagctgaagcccggcgagagcgccttcatcgccgccaacgagagccccgtgaccgtgaagggccacggccgcctggcccgcgtgtacaacaagctgtgataggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccgcaattgaagtttgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 4 - DNA, 20 bp, synthetic construct
  misc_feature 1..20: note = for editing GW2-2
  source 1..20: mol_type = other DNA
aagctcgcgccctgctaccc

SEQ ID NO: 5 - DNA, 19617 bp, synthetic construct
  misc_feature 1..19617: note = vector 22808
  misc_feature 4..259: note = bNRB-05
  regulatory 304..2100: note = promoter - prSoUbi4-04; regulatory_class = promoter
  gene 2119..5193: note = cTNPLAIIAFw-01
  regulatory 5200..5452: note = terminator - tNOS-05-01; regulatory_class = terminator
  regulatory 5486..7478: note = promoter - prUbi1-10; regulatory_class = promoter
  gene 7492..10566: note = cTNPLAIIARv-01
  regulatory 10573..10825: note = terminator - tNOS-05-01; regulatory_class = terminator
  regulatory 10844..12835: note = promoter - prUbi1-04; regulatory_class = promoter
  gene 12852..14030: note = cPMI-09
  regulatory 14053..14305: note = terminator - tNOS-05-01; regulatory_class = terminator
  misc_feature 14349..14478: note = bNLB-03
  gene 14758..15546: note = cSpec-03
  regulatory 15641..15771: note = promoter - prVirG-01; regulatory_class = promoter
  gene 15846..16571: note = cVirG-01
  gene 16601..17674: note = cRepA-01
  misc_feature 17717..18121: note = oVS1-02
  misc_feature 18799..19605: note = oCOLE-06
  source 1..19617: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtaccattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggatcgcggccgcgccaccatgggaaaacctattcctaatcctctgctgggcctggattctaccggaggcatggcccctaagaaaaagcggaaggtggacggcggagtggacctgagaacactgggatattctcagcagcagcaggagaagatcaagcccaaggtgagatctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacagtggtctggagccagagccctggaagccctgctgacagtggccggagaactgagaggacctcctctgcagctggatacaggacagctgctgaagattgccaaaaggggcggagtgaccgcggtggaagccgtgcacgcctggagaaatgccctgacaggagcccctctgaacctgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggactgacccccgaacaggtggtggccattgccagcaacaacggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacgggctgacccccgaacaggtggtggccattgccagcaacggcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggcctcactcccgaacaggtggtggccattgccagcaacatcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggactgacccccgaacaggtggtggccattgccagcaacggcggcggcaagcaggccctggaaaccgtgcagagactgctgcccgtgctgtgccaggcccatggcctgacacctgaacaggtggtggctatcgcctctaacaacggaggaaaacaagcactcgagacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacgggctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccacagcaggtcgtggcaattgctagcaatatcggcggacggcccgccctggagagcattgtggcccagctgtctagacctgatcctgccctggccgccctgacaaatgatcacctggtggccctggcctgtctgggaggcagacctgccctggatgccgtgaaaaaaggactgcctcacgcccctgccctgattaaaagaacaaatagaagaatccccgagcggacctctcacagagtggccggatcccagctggtgaaatctgagctggaggagaagaagtctgagctgagacacaagctgaagtacgtgcctcacgagtacatcgagctgatcgagatcgccagaaatagcacccaggatagaatcctggagatgaaggtgatggagttcttcatgaaagtgtacggctacagaggaaagcatctgggaggaagcagaaaacctgacggagccatttatacagtgggcagccctatcgattatggcgtgatcgtggatacaaaggcctacagcggaggctacaatctgcctattggacaggccgatgagatgcagagatacgtggaggagaaccaaaccaggaacaagcatatcaaccctaacgagtggtggaaggtgtacccttctagcgtgaccgagttcaagttcctgtttgtgagcggccacttcaagggcaattataaggcccagctgaccaggctgaaccacatcacaaattgtaatggcgccgtgctgtctgtggaggaactgctgattggaggagagatgattaaggccggaacactgacactggaggaggtgagaagaaagttcaacaacggcgagatcaacttctgaaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaaccctagtcgaagacaaccggtgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagcggccgcgccaccatgggaaaacctattcctaatcctctgctgggcctggattctaccggaggcatggcccctaagaaaaagcggaaggtggacggcggagtggacctgagaacactgggatattctcagcagcagcaggagaagatcaagcccaaggtgagatctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacagtggtctggagccagagccctggaagccctgctgacagtggccggagaactgagaggacctcctctgcagctggatacaggacagctgctgaagattgccaaaaggggcggagtgaccgcggtggaagccgtgcacgcctggagaaatgccctgacaggagcccctctgaacctgacccccgaacaggtggtggccattgccagcaacaacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacggactgacccccgaacaggtggtggccattgccagcaacggcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacgggctgacccccgaacaggtggtggccattgccagcaacggcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggcctcactcccgaacaggtggtggccattgccagcaacaacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggaggaaaacaagcactcgagacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacgggctgacccccgaacaggtggtggccattgccagccacgacggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccacagcaggtcgtggcaattgctagccacgacggcggacggcccgccctggagagcattgtggcccagctgtctagacctgatcctgccctggccgccctgacaaatgatcacctggtggccctggcctgtctgggaggcagacctgccctggatgccgtgaaaaaaggactgcctcacgcccctgccctgattaaaagaacaaatagaagaatccccgagcggacctctcacagagtggccggatcccagctggtgaaatctgagctggaggagaagaagtctgagctgagacacaagctgaagtacgtgcctcacgagtacatcgagctgatcgagatcgccagaaatagcacccaggatagaatcctggagatgaaggtgatggagttcttcatgaaagtgtacggctacagaggaaagcatctgggaggaagcagaaaacctgacggagccatttatacagtgggcagccctatcgattatggcgtgatcgtggatacaaaggcctacagcggaggctacaatctgcctattggacaggccgatgagatgcagagatacgtggaggagaaccaaaccaggaacaagcatatcaaccctaacgagtggtggaaggtgtacccttctagcgtgaccgagttcaagttcctgtttgtgagcggccacttcaagggcaattataaggcccagctgaccaggctgaaccacatcacaaattgtaatggcgccgtgctgtctgtggaggaactgctgattggaggagagatgattaaggccggaacactgacactggaggaggtgagaagaaagttcaacaacggcgagatcaacttctgaaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaagacggaccgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccggcagcagccatgcagaagctgatcaacagcgtgcagaactacgcctggggcagcaagaccgccctgaccgagctgtacggcatggagaaccccagcagccagcccatggccgagctgtggatgggcgcccaccccaagagcagcagccgcgtgcagaacgccgccggcgacatcgtgagcctgcgcgacgtgatcgagagcgacaagagcaccctgctgggcgaggccgtggccaagcgcttcggcgagctgcccttcctgttcaaggtgctgtgcgccgcccagcccctgagcatccaggtgcaccccaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgtgagcctgctgcagcccgtggccggcgcccaccccgccatcgcccacttcctgcagcagcccgacgccgagcgcctgagcgagctgttcgccagcctgctgaacatgcagggcgaggagaagagccgcgccctggccatcctgaagagcgccctggacagccagcagggcgagccctggcagaccatccgcctgatcagcgagttctaccccgaggacagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggccaagcccgccaaccagctgctgacccagcccgtgaagcagggcgccgagctggacttccccatccccgtggacgacttcgccttcagcctgcacgacctgagcgacaaggagaccaccatcagccagcagagcgccgccatcctgttctgcgtggagggcgacgccaccctgtggaagggcagccagcagctgcagctgaagcccggcgagagcgccttcatcgccgccaacgagagccccgtgaccgtgaagggccacggccgcctggcccgcgtgtacaacaagctgtgataggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccgcaattgaagtttgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 6 - DNA, 55 bp, synthetic construct
  misc_feature 1..55: note = target sequence for the TALEN of 22808
  source 1..55: mol_type = other DNA
tccagggtcaacgtggagacagggaggtacgaaccggtgactggcgaaggaagca

SEQ ID NO: 7 - DNA, 15722 bp, synthetic construct
  misc_feature 1..15722: note = vector 23123
  misc_feature 4..259: note = bNRB-05
  regulatory 304..2100: note = promoter - prSoUbi4-04; regulatory_class = promoter
  gene 2117..6286: note = cCas9-01
  variation 5606..5608: note = mutation - L to V mutation
  variation 5651..5653: note = mutation - I to V mutation
  regulatory 6292..6544: note = terminator - tNOS-05-01; regulatory_class = terminator
  regulatory 6551..6925: note = promoter - prOsU3-01; regulatory_class = promoter
  misc_feature 6927..6946: note = xZmPLAIIA
  misc_feature 6927..7031: note = rsgRNAZmPLAIIA02
  regulatory 7042..9033: note = promoter - prUbi1-04; regulatory_class = promoter
  gene 9050..10228: note = cPMI-09
  regulatory 10251..10503: note = terminator - tNOS-05-01; regulatory_class = terminator
  misc_feature 10547..10676: note = bNLB-03
  gene 10956..11744: note = cSpec-03
  regulatory 11839..11969: note = promoter - prVirG-01; regulatory_class = promoter
  gene 12706..13779: note = cRepA-01
  misc_feature 13822..14226: note = oVS1-02
  misc_feature 14904..15710: note = oCOLE-06
  source 1..15722: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtaccattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggatcgcggccgctcatatggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggacccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaagggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcagggtcaacgtggagacaggggttttagagctagaaatagcaagttaaaataaggctagtccgttatcaacttgaaaaagtggcaccgagtcggtgctttttttttcggaccgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccggcagcagccatgcagaagctgatcaacagcgtgcagaactacgcctggggcagcaagaccgccctgaccgagctgtacggcatggagaaccccagcagccagcccatggccgagctgtggatgggcgcccaccccaagagcagcagccgcgtgcagaacgccgccggcgacatcgtgagcctgcgcgacgtgatcgagagcgacaagagcaccctgctgggcgaggccgtggccaagcgcttcggcgagctgcccttcctgttcaaggtgctgtgcgccgcccagcccctgagcatccaggtgcaccccaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgtgagcctgctgcagcccgtggccggcgcccaccccgccatcgcccacttcctgcagcagcccgacgccgagcgcctgagcgagctgttcgccagcctgctgaacatgcagggcgaggagaagagccgcgccctggccatcctgaagagcgccctggacagccagcagggcgagccctggcagaccatccgcctgatcagcgagttctaccccgaggacagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggccaagcccgccaaccagctgctgacccagcccgtgaagcagggcgccgagctggacttccccatccccgtggacgacttcgccttcagcctgcacgacctgagcgacaaggagaccaccatcagccagcagagcgccgccatcctgttctgcgtggagggcgacgccaccctgtggaagggcagccagcagctgcagctgaagcccggcgagagcgccttcatcgccgccaacgagagccccgtgaccgtgaagggccacggccgcctggcccgcgtgtacaacaagctgtgataggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccgcaattgaagtttgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 8 - DNA, 20 bp, synthetic construct
  misc_feature 1..20: note = for editing MTL
  source 1..20: mol_type = other DNA
gggtcaacgtggagacaggg

SEQ ID NO: 9 - DNA, 41 bp, Zea mays
  source 1..41: mol_type = genomic DNA
agggtcaacgtggagacagggaggtacgaaccggtgactgg

SEQ ID NO: 10 - DNA, 45 bp, Zea mays
  source 1..45: mol_type = genomic DNA
agggtcaacgtggagacaggcgaggaggtacgaaccggtgactgg

SEQ ID NO: 11 - DNA, 42 bp, synthetic construct
  misc_feature 1..42: note = mutated MTL
  source 1..42: mol_type = other DNA
agggtcaacgtggagacaagggaggtacgaaccggtgactgg

SEQ ID NO: 12 - DNA, 28 bp, synthetic construct
  misc_feature 1..28: note = mutated MTL portion
  source 1..28: mol_type = other DNA
agggtcaacgtggagaaccggtgactgg

SEQ ID NO: 13 - DNA, 40 bp, synthetic construct
  misc_feature 1..40: note = mutated MTL portion
  source 1..40: mol_type = other DNA
agggtcaacgtggagacgggaggtacgaaccggtgactgg

SEQ ID NO: 14 - DNA, 28 bp, synthetic construct
  misc_feature 1..28: note = mutated MTL portion
  source 1..28: mol_type = other DNA
agggtcaacgtggagaaccggtgactgg

SEQ ID NO: 15 - DNA, 42 bp, synthetic construct; identical to SEQ ID NO: 11
  misc_feature 1..42: note = mutated MTL portion
  source 1..42: mol_type = other DNA

SEQ ID NO: 16 - DNA, 40 bp, synthetic construct; identical to SEQ ID NO: 13
  misc_feature 1..40: note = mutated MTL portion
  source 1..40: mol_type = other DNA

SEQ ID NO: 17 - DNA, 41 bp, synthetic construct; identical to SEQ ID NO: 9
  misc_feature 1..41: note = unmutated MTL portion
  source 1..41: mol_type = other DNA

SEQ ID NO: 18 - DNA, 28 bp, synthetic construct
  misc_feature 1..28: note = mutated MTL portion
  source 1..28: mol_type = other DNA
agggtcaacgtggagaaccggtgactgg

SEQ ID NO: 19 - DNA, 1371 bp, Zea mays
  source 1..1371: mol_type = genomic DNA
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacagggaggtacgaaccggtgactggcgaaggaagcaatgccgatgccctcggtgggctcgctaggcagctctccgaggagaggagaacaaggctcgcgcgccgcgtgtctgccatcaacccaagaggctctagatgtgcgtcgtacgatatctaagacaagtggctttactgtcagtcacatgcttgtaaataagtagactttattttaataaaacataaaaatatatat

SEQ ID NO: 20 - DNA, 15722 bp, synthetic construct
  misc_feature 1..15722: note = vector 23397
  misc_feature 4..259: note = bNRB-05
  regulatory 304..2100: note = promoter - prSoUbi4-04; regulatory_class = promoter
  gene 2117..6286: note = cCas9-01
  variation 5606..5608: note = mutation - L to V mutation
  variation 5651..5653: note = mutation - I to V mutation
  regulatory 6292..6544: note = terminator - tNOS-05-01; regulatory_class = terminator
  regulatory 6551..6925: note = promoter - prOsU3-01; regulatory_class = promoter
  misc_feature 6927..6946: note = xZmVLHP
  misc_feature 6927..7031: note = rsgRNAZmVLHP-02
  regulatory 7042..9033: note = promoter - prUbi1-04; regulatory_class = promoter
  gene 9050..10228: note = cPMI-09
  regulatory 10251..10503: note = terminator - tNOS-05-01; regulatory_class = terminator
  misc_feature 10547..10676: note = bNLB-03
  gene 10956..11744: note = cSpec-03
  regulatory 11839..11969: note = promoter - prVirG-01; regulatory_class = promoter
  gene 12706..13779: note = cRepA-01
  misc_feature 13822..14226: note = oVS1-02
  misc_feature 14904..15710: note = oCOLE-06
  source 1..15722: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtaccattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggatcgcggccgctcatatggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggacccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaagggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcagctggagctgagcttccggggttttagagctagaaatagcaagttaaaataaggctagtccgttatcaacttgaaaaagtggcaccgagtcggtgctttttttttcggaccgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccggcagcagccatgcagaagctgatcaacagcgtgcagaactacgcctggggcagcaagaccgccctgaccgagctgtacggcatggagaaccccagcagccagcccatggccgagctgtggatgggcgcccaccccaagagcagcagccgcgtgcagaacgccgccggcgacatcgtgagcctgcgcgacgtgatcgagagcgacaagagcaccctgctgggcgaggccgtggccaagcgcttcggcgagctgcccttcctgttcaaggtgctgtgcgccgcccagcccctgagcatccaggtgcaccccaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgtgagcctgctgcagcccgtggccggcgcccaccccgccatcgcccacttcctgcagcagcccgacgccgagcgcctgagcgagctgttcgccagcctgctgaacatgcagggcgaggagaagagccgcgccctggccatcctgaagagcgccctggacagccagcagggcgagccctggcagaccatccgcctgatcagcgagttctaccccgaggacagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggccaagcccgccaaccagctgctgacccagcccgtgaagcagggcgccgagctggacttccccatccccgtggacgacttcgccttcagcctgcacgacctgagcgacaaggagaccaccatcagccagcagagcgccgccatcctgttctgcgtggagggcgacgccaccctgtggaagggcagccagcagctgcagctgaagcccggcgagagcgccttcatcgccgccaacgagagccccgtgaccgtgaagggccacggccgcctggcccgcgtgtacaacaagctgtgataggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccgcaattgaagtttgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 21 - DNA, 20 bp, synthetic construct
  misc_feature 1..20: note = for editing VLHP2
  source 1..20: mol_type = other DNA
gctggagctgagcttccggg

SEQ ID NO: 22 - DNA, 15722 bp, synthetic construct
  misc_feature 1..15722: note = vector 23398
  misc_feature 4..259: note = bNRB-05
  regulatory 304..2100: note = promoter - prSoUbi4-04; regulatory_class = promoter
  gene 2117..6286: note = cCas9-01
  misc_feature 5606..5608: note = L to V mutation
  misc_feature 5651..5653: note = I to V mutation
  regulatory 6292..6544: note = terminator - tNOS-05-01; regulatory_class = terminator
  regulatory 6551..6925: note = promoter - prOsU3-01; regulatory_class = promoter
  misc_feature 6927..6946: note = xZmGW2
  misc_feature 6927..7031: note = rsgRNAZmGW2-01
  regulatory 7042..9033: note = promoter - prUbi1-04; regulatory_class = promoter
  gene 9050..10228: note = cPMI-09
  regulatory 10251..10503: note = terminator - tNOS-05-01; regulatory_class = terminator
  misc_feature 10547..10676: note = bNLB-03
  gene 10956..11744: note = cSpec-03
  regulatory 11839..11969: note = promoter - prVirG-01; regulatory_class = promoter
  gene 12706..13779: note = cRepA-01
  misc_feature 13822..14226: note = oVS1-02
  misc_feature 14904..15710: note = oCOLE-06
  source 1..15722: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtaccattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggatcgcggccgctcatatggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggacccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaagggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcagagcggttcacgcggccgcagttttagagctagaaatagcaagttaaaataaggctagtccgttatcaacttgaaaaagtggcaccgagtcggtgctttttttttcggaccgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccggcagcagccatgcagaagctgatcaacagcgtgcagaactacgcctggggcagcaagaccgccctgaccgagctgtacggcatggagaaccccagcagccagcccatggccgagctgtggatgggcgcccaccccaagagcagcagccgcgtgcagaacgccgccggcgacatcgtgagcctgcgcgacgtgatcgagagcgacaagagcaccctgctgggcgaggccgtggccaagcgcttcggcgagctgcccttcctgttcaaggtgctgtgcgccgcccagcccctgagcatccaggtgcaccccaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgtgagcctgctgcagcccgtggccggcgcccaccccgccatcgcccacttcctgcagcagcccgacgccgagcgcctgagcgagctgttcgccagcctgctgaacatgcagggcgaggagaagagccgcgccctggccatcctgaagagcgccctggacagccagcagggcgagccctggcagaccatccgcctgatcagcgagttctaccccgaggacagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggccaagcccgccaaccagctgctgacccagcccgtgaagcagggcgccgagctggacttccccatccccgtggacgacttcgccttcagcctgcacgacctgagcgacaaggagaccaccatcagccagcagagcgccgccatcctgttctgcgtggagggcgacgccaccctgtggaagggcagccagcagctgcagctgaagcccggcgagagcgccttcatcgccgccaacgagagccccgtgaccgtgaagggccacggccgcctggcccgcgtgtacaacaagctgtgataggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccgcaattgaagtttgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 23 - DNA, 20 bp, synthetic construct
  misc_feature 1..20: note = for editing GW2-1
  source 1..20: mol_type = other DNA
gagcggttcacgcggccgca

SEQ ID NO: 24 - DNA, 15721 bp, synthetic construct
  misc_feature 1..15721: note = vector 23763
  misc_feature 4..259: note = bNRB-05
  regulatory 304..2100: note = promoter - prSoUbi4-04; regulatory_class = promoter
  gene 2117..6286: note = cCas9-01
  variation 5606..5608: note = mutation - L to V mutation
  variation 5651..5653: note = mutation - I to V mutation
  misc_feature 6221..6283: note = xSV40NLS-03
  regulatory 6292..6544: note = terminator - tNOS-05-01; regulatory_class = terminator
  regulatory 6551..6925: note = promoter - prOsU3-01; regulatory_class = promoter
  misc_feature 6927..6945: note = xTaVLHP1
  misc_feature 6927..7030: note = rsgRNA TaVLHP1-01
  regulatory 7041..9032: note = promoter - prUbi1-04; regulatory_class = promoter
  gene 9049..10227: note = cPMI-09
  regulatory 10250..10502: note = terminator - tNOS-05-01; regulatory_class = terminator
  misc_feature 10546..10675: note = bNLB-03
  gene 10955..11743: note = cSpec-03
  regulatory 11838..11968: note = promoter - prVirG-01; regulatory_class = promoter
  gene 12705..13778: note = cRepA-01
  misc_feature 13821..14225: note = oVS1-02
  misc_feature 14903..15709: note = oCOLE-06
  source 1..15721: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtaccattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggatcgcggccgctcatatggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggacccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaagggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcagacgagcaggcgcagttccgttttagagctagaaatagcaagttaaaataaggctagtccgttatcaacttgaaaaagtggcaccgagtcggtgctttttttttcggaccgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccggcagcagccatgcagaagctgatcaacagcgtgcagaactacgcctggggcagcaagaccgccctgaccgagctgtacggcatggagaaccccagcagccagcccatggccgagctgtggatgggcgcccaccccaagagcagcagccgcgtgcagaacgccgccggcgacatcgtgagcctgcgcgacgtgatcgagagcgacaagagcaccctgctgggcgaggccgtggccaagcgcttcggcgagctgcccttcctgttcaaggtgctgtgcgccgcccagcccctgagcatccaggtgcaccccaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgtgagcctgctgcagcccgtggccggcgcccaccccgccatcgcccacttcctgcagcagcccgacgccgagcgcctgagcgagctgttcgccagcctgctgaacatgcagggcgaggagaagagccgcgccctggccatcctgaagagcgccctggacagccagcagggcgagccctggcagaccatccgcctgatcagcgagttctaccccgaggacagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggccaagcccgccaaccagctgctgacccagcccgtgaagcagggcgccgagctggacttccccatccccgtggacgacttcgccttcagcctgcacgacctgagcgacaaggagaccaccatcagccagcagagcgccgccatcctgttctgcgtggagggcgacgccaccctgtggaagggcagccagcagctgcagctgaagcccggcgagagcgccttcatcgccgccaacgagagccccgtgaccgtgaagggccacggccgcctggcccgcgtgtacaacaagctgtgataggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccgcaattgaagtttgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 25 - DNA, 19 bp, synthetic construct
  misc_feature 1..19: note = for editing VLHP1 in wheat
  source 1..19: mol_type = other DNA
gacgagcaggcgcagttcc

SEQ ID NO: 26 - DNA, 20 bp, Triticum aestivum
  source 1..20: mol_type = genomic DNA
gctggagctgagcttccggg

SEQ ID NO: 27 - DNA, 20 bp, Triticum aestivum
  source 1..20: mol_type = genomic DNA
tctggagctgagcttccggg

SEQ ID NO: 28 - DNA, 20 bp, Zea mays
  source 1..20: mol_type = genomic DNA
aggcgtcgagcagcgaggtg

SEQ ID NO: 29 - DNA, 20 bp, synthetic construct
  misc_feature 1..20: note = edited ZmVLHP-03 portion
  source 1..20: mol_type = other DNA
aggcgttgagcagcgaggtg

SEQ ID NO: 30 - DNA, 75 bp, synthetic construct
  misc_feature 1..75: note = repair donor template for creating E149L mutation in ZmPYL-D
  source 1..75: mol_type = other DNA
ccttggtgttgccgtcggggacgtcgacgacgaatgacaggatgacgagcgtccctggccggccgtcgatgacct

SEQ ID NO: 31 - DNA, 15722 bp, synthetic construct
  misc_feature 1..15722: note = vector 23136
  misc_feature 4..259: note = bNRB-05
  regulatory 304..2100: note = promoter - prSoUbi4-04; regulatory_class = promoter
  gene 2117..6286: note = cCas9-01
  variation 5606..5608: note = mutation - L to V mutation
  variation 5651..5653: note = mutation - I to V mutation
  regulatory 6292..6544: note = terminator - tNOS-05-01; regulatory_class = terminator
  regulatory 6551..6925: note = promoter - prOsU3-01; regulatory_class = promoter
  misc_feature 6927..6946: note = xZmPYL-D
  misc_feature 6927..7031: note = rsgRBAZmPYLd-02
  regulatory 7042..9033: note = promoter - prUbi1-04; regulatory_class = promoter
  gene 9050..10228: note = cPMI-09
  regulatory 10251..10503: note = terminator - tNOS-05-01; regulatory_class = terminator
  misc_feature 10547..10676: note = bNLB-03
  gene 10956..11744: note = cSpec-03
  regulatory 11839..11969: note = promoter - prVirG-01; regulatory_class = promoter
  gene 12706..13779: note = cRepA-01
  misc_feature 13822..14226: note = oVS1-02
  misc_feature 14904..15710: note = oCOLE-06
  source 1..15722: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtaccattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggatcgcggccgctcatatggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggacccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaagggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcagtcggggacgtcgacgacgagttttagagctagaaatagcaagttaaaataaggctagtccgttatcaacttgaaaaagtggcaccgagtcggtgctttttttttcggaccgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccggcagcagccatgcagaagctgatcaacagcgtgcagaactacgcctggggcagcaagaccgccctgaccgagctgtacggcatggagaaccccagcagccagcccatggccgagctgtggatgggcgcccaccccaagagcagcagccgcgtgcagaacgccgccggcgacatcgtgagcctgcgcgacgtgatcgagagcgacaagagcaccctgctgggcgaggccgtggccaagcgcttcggcgagctgcccttcctgttcaaggtgctgtgcgccgcccagcccctgagcatccaggtgcaccccaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgtgagcctgctgcagcccgtggccggcgcccaccccgccatcgcccacttcctgcagcagcccgacgccgagcgcctgagcgagctgttcgccagcctgctgaacatgcagggcgaggagaagagccgcgccctggccatcctgaagagcgccctggacagccagcagggcgagccctggcagaccatccgcctgatcagcgagttctaccccgaggacagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggccaagcccgccaaccagctgctgacccagcccgtgaagcagggcgccgagctggacttccccatccccgtggacgacttcgccttcagcctgcacgacctgagcgacaaggagaccaccatcagccagcagagcgccgccatcctgttctgcgtggagggcgacgccaccctgtggaagggcagccagcagctgcagctgaagcccggcgagagcgccttcatcgccgccaacgagagccccgtgaccgtgaagggccacggccgcctggcccgcgtgtacaacaagctgtgataggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccgcaattgaagtttgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 32 - DNA, 20 bp, synthetic construct
  misc_feature 1..20: note = encoding gRNA for vector 23136
  source 1..20: mol_type = other DNA
gtcggggacgtcgacgacga

SEQ ID NO: 33 - DNA, 1823 bp, Oryza sativa
  source 1..1823: mol_type = genomic DNA
acagtgactagtgacaaacgatcgatcgatccctccatccacaaaccctcctcgatctcatcttccttcgtctcgtcaatggcggcgagctactcgtgccggcggacatgcgaggcgtgcagcacgagggcgatggccgggtgcgtggtgggcgagccggcgtcggcgccggggcagcgggtgacgttgctggcgatcgacggcggcggcatcaggggcctcatcccgggcaccatcctcgccttcctcgaggccaggctgcaggagctggatggccccgacgcgcgcctcgccgattacttcgactgcatcgccgggaccagcaccggcggcctcatcaccgccatgctcgccgcgcccggcgaccacggccgcccgctcttcgccgccagcgacatcaaccgcttctacctcgacaacggcccactcatcttcccacaaaagtaactgatcacctcgaattcgatctcctctcttcgatctctgcattatttgatttgattggggattgtgggcggcgtggcgtggcgtccaggaggtgcggcatggcggcggccatggcggcgctgacgaggccgaggtacaacggcaagtacctgcaggggaagatcaggaagatgctgggcgagacgagggtgcgcgacacgctgacgaacgtcgtcatccccacgttcgacgtcaggctgctccagccaaccatcttctccacatacgacgtgcgtgcgttgattccatccgcattggcgttggaatcagctgattgtttgattgatcgaacaattgatcggttaaaattttgcaggcgaagagcatgccgctcaagaacgcgctcctctccgacatctgcatcagcacatccgcggcgccgacctacctccccgcgcactgcttccagaccaccgacgacgccaccggcaaggtccgcgagttcgacctcatcgacggcggcgtcgccgccaacaacccggtaactaatcaatcaagcaatccatcaaacgaagatccacatgtgcattcctgtggtacaaatgctgatcgatcgatggatggatcgattttcgcgagaacgtacagacgatggtggccatgacgcagatcaccaagaagataatggtgaaggacaaggaggagctgtacccggtaaagccgtcggactgcggtaagttcctggtgctgtccgtgggcaccgggtcgacgtcggaccaggggatgtacacggcgaggcagtgctcgcggtgggggatcgtccggtggctgcgcaacaaggggatggcgcccatcatcgacatcttcatggcggccagctccgacctcgtcgacatccacgccgccgtcatgttccagtcgctgcacagcgacggcgactacctccgcatccaggacaacacgctccacggcgacgccgccacggtggacgccgccaccagggacaacatgcgggcgctcgtcgggatcggcgagcggatgctggcgcagcgggtgtcgagggtcaacgtcgagaccggcaggtacgtcgaggtgcccggcgccggcagcaacgccgacgcgctgaggggcttcgccaggcagctctccgaggagaggagggcgaggctaggtcggcgaaacgcctgcggcggcggcggcgaaggagagcccagcggcgtggcgtgcaagcgttagtaactgtacacgcatcatgctgacgcgatcttttttatttttctttttttttttttacctttctagcggacatggggaataacaagacgtgacagtagtgcaatcggtttgtaacgtgcgtataccaacattgatccatttcttcatcacagtttcagttc

SEQ ID NO: 34 - DNA, 15921 bp, synthetic construct
  misc_feature 1..15921: note = Vector 24038
  misc_feature 4..259: note = bNRB-05
  regulatory 313..1149: note = promoter - prZmGRMZM5G876285-01; regulatory_class = promoter
  gene 1152..5412: note = cCas9-12
  regulatory 5419..6736: note = terminator - tZmGRMZM5G876285-01; regulatory_class = terminator
  regulatory 6750..7124: note = promoter - prOsU3-01; regulatory_class = promoter
  misc_feature 7126..7145: note = xZmVLHP2
  misc_feature 7126..7230: note = rsgRNAZmVLHP-02
  misc_feature 7146..7157: note = rCrRNA-01
  misc_feature 7146..7230: note = rsgRNAbase-01
  misc_feature 7162..7230: note = rTracrRNA-01
  regulatory 7241..9232: note = promoter - prUbi-04; regulatory_class = promoter
  gene 9249..10427: note = cPMI-09
  regulatory 10450..10702: note = terminator - tNOS-05-01; regulatory_class = terminator
  misc_feature 10746..10875: note = bNLB-03
  gene 11155..11943: note = cSpec-03
  regulatory 12038..12168: note = promoter - prVirG-01; regulatory_class = promoter
  gene 12243..12875: note = cVirG-09
  gene 12905..13978: note = cRepA-01
  misc_feature 14021..14425: note = oVA1-02
  misc_feature 15103..15909: note = oCOLE-06
  source 1..15921: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtaccgggaccctaagtaatcttgtgctacaaatttatttttcagacagaaaaatctatttagctaactaattaatacaaattaataccaagcaacgatagatgaacatctagttgtctaattagctaactaattaatacaaattaagtagaatccttaccgtggggagatggggcgcgacgaagtgctcgagcttggggcgcggcgaccggcgacgtgaagcttgggggcgcgggggccggacggcgctgcgggcggcatggcgggcggctgcgggcggcggcgcgggcgcaggaaacaaacgacgggagtgggaggaaggagaaagcggcgcgccggtttagtcctagctcggcgccaagatctgtggcgccgagctaggtgccacgatggccgccgcgtcagcaaagctcggcgccaaggcatgttgcgccgagccgtgttagctcggcgtcatagctcatggtgccgagttttgggtctaaaattgcgtttaagtattctagggatctaaacgcaaatatttttcgaaaatagggccgaaaaacaaaaaaaaatcggtcgtttcgtcgagcacatcgtccagcctatcttgcatgtccatcctctctatggttcgcgagccgcgcgcatggcgctccaaaggaggggcgaggttgaatatagacagatggaatgggtggttctctatttatagcgcatgcagtcgtcccctggcacacctatttatatgtgagcgttcctggcactagagagatcgatcgatcgagcttaattgcgccactgctcgttatcctcctcttgcattgcattgcaggtcgtagttgagcagcagcaaccactgcacaggccatggacaagaagtacagcatcggcctggacatcggcaccaacagcgtgggctgggccgtgatcaccgacgagtacaaggtgataccaatttgcatgatccttgttcgttctagctcttgcatgccgatcagttgaatcacgcggtttccttctgcgcatttgcatccaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggacccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgagctctaatgcatccaaacaacgacaccaacgccaacattaattaattagtagtctccatgccctgggattgtgcgtggccgctccgttgaacaccacccatccttcgttcggcattttttccccccttgtttatataattttattgtatcgttttggcaaataattttgtgattcgaccccaaagcaagtttggttgtcttacgatttgtaaacctggaacaatatataatgtgattgaactgctttgtctattctttttgtagtacgataatatgtatatgtattccatgcgatctcttctagggcgacgactaatgtgcaagtgtgtgtttgcatgcgctgagcacggagtttgtattcaggggtcaatatctttcgattcctttatctaaaaaggtgttgcatatatctaaaaaaaagaaaaaaaaggcttacaactgttgaaaaaataagcatttttagttttaatttaattcagaaaatcatagtgatatatgtgacgatatgcatgtgcatatgtatcactactcacataaacagtaaacaacagtaaaatatgtataaatacaaaaataacaaagtgtaccctgcggagggaccgatgttcaaggcatctgtggctccattcacacgagacatctcgtgtgtatgttcgatgtagtcatacgcagtcgaggcagtcagatgtacgcagtgcagtccctcgatcggcgccggcgacgaggaacttgatcagtgctggtcgagcggacgaagcgagcagtcgcgagtacgctcccgaaaaacatgatcgctcgcacacccatgcaagtgtcgctctgcggacgacgatttcggaagcctacgcgtatgagaatgtttgtatgtgtgttctctcgtaaccagaagcctcatctcctccgtatatatacacgcgcagagggaggccaacagatagtaacggtggaaggaatactcggaccaaggtccgatctaccatggccacggcccggcctggccagcggcgcgtgcgtgtggcagtccttcatccttttatcagcttatcaatagatgcaccaaagatccacctatttaagttgattgaattgtctcttgtacttccggtatgttactaaagtaataatacaccgtagcattaaattgggcctttagcattggctattattgaatattaatttgagccagacccaccaccagatgctaagtcacaccaaaatgctctcatcatctcaaacatttcatatactggtgtttcgatggagactattaagttgaacatccacctagaatctagattacacttgaccacaactacataatggacggaccgttcgaagggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcagctggagctgagcttccggggttttagagctagaaatagcaagttaaaataaggctagtccgttatcaacttgaaaaagtggcaccgagtcggtgctttttttttcggaccgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccggcagcagccatgcagaagctgatcaacagcgtgcagaactacgcctggggcagcaagaccgccctgaccgagctgtacggcatggagaaccccagcagccagcccatggccgagctgtggatgggcgcccaccccaagagcagcagccgcgtgcagaacgccgccggcgacatcgtgagcctgcgcgacgtgatcgagagcgacaagagcaccctgctgggcgaggccgtggccaagcgcttcggcgagctgcccttcctgttcaaggtgctgtgcgccgcccagcccctgagcatccaggtgcaccccaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgtgagcctgctgcagcccgtggccggcgcccaccccgccatcgcccacttcctgcagcagcccgacgccgagcgcctgagcgagctgttcgccagcctgctgaacatgcagggcgaggagaagagccgcgccctggccatcctgaagagcgccctggacagccagcagggcgagccctggcagaccatccgcctgatcagcgagttctaccccgaggacagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggccaagcccgccaaccagctgctgacccagcccgtgaagcagggcgccgagctggacttccccatccccgtggacgacttcgccttcagcctgcacgacctgagcgacaaggagaccaccatcagccagcagagcgccgccatcctgttctgcgtggagggcgacgccaccctgtggaagggcagccagcagctgcagctgaagcccggcgagagcgccttcatcgccgccaacgagagccccgtgaccgtgaagggccacggccgcctggcccgcgtgtacaacaagctgtgataggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccgcaattgaagtttgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 35 - DNA, 17954 bp, synthetic construct
  misc_feature 1..17954: note = vector 24039
  misc_feature 4..259: note = bNRB-05
  regulatory 315..1729: note = promoter - prZmGRMZM2G020852-01; regulatory_class = promoter
  gene 1731..5979: note = cCas9-13
  regulatory 5989..8769: note = terminator - tZmGRMZM2G020852-01; regulatory_class = terminator
  regulatory 8783..9157: note = promoter - prOsU3-01; regulatory_class = promoter
  misc_feature 9159..9178: note = xZmVLHP2
  misc_feature 9159..9263: note = rsgRNAZmVLHP-02
  misc_feature 9179..9190: note = rCrRNA-01
  misc_feature 9195..9263: note = rTracrRNA-01
  regulatory 9274..11265: note = promoter - prUbi1-04; regulatory_class = promoter
  gene 11282..12460: note = cPMI-09
  regulatory 12483..12735: note = terminator - tNOS-05-01; regulatory_class = terminator
  misc_feature 12779..12908: note = bNLB-03
  gene 13188..13976: note = cSpec-03
  regulatory 14071..14201: note = promoter - prVirG-01; regulatory_class = promoter
  gene 14276..14908: note = cVirG-09
  gene 14938..16011: note = cRepA-01
  misc_feature 16054..16458: note = oVS1-02
  misc_feature 17136..17942: note = oCOLE-o6
  source 1..17954: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtaccggaccgttataacagtgaatacaaaaatgacattcgtgttatttagcacaagttacgatctatttcaggaacatgccggaattttcgaacaccattctcacaaaacatgaccttgaacttgcgatccagttgttttaaaattatataaaacaaaaacaaagtcagaaaatcatgaaacttgtcgacatgtcatgatatcatatgtagagactctaataaaaagttgagattgtttcatgaaagttgtcacacactatgtgtagaaacttagcccgtctacattgaagttctatgatttcatgtgaaggacacctaggcatcgatgtttatgataatatcttatgtttgtttggacaaaatattaaaaacaaataaaaggggtccctgatcactttgacgagcattgcattcagcaaagggtgcctttgttgagtgcaatggtcatagaactcggtagaaaagacatacataaacatcgggaaacttgctttaccgcacgctatggccaagacactcggcaaactaggctcctttgttgagtgccatctcaagcactcgacattggaactacgactaggcctcacggaagctttctttgccgagtgccactaagcgaggaactcggacactcagcaacagctctgtcatcgtcacgatgtcttttctttgtcgtgtaccagttggcactcggttaagactttactgagtgcccgatagaaagtactcggcaaagagaccgttgccgacgtttggttcactgagggctctttgctgccttttggacttgacaaagaagtcatctccagtactgtctcctaggacgcaggatttatgttttttcccggagctcgatctgtgggacatcacagatggtccaatctggtgatctaaaatggacggtttgccaagcccacagagaagtctttaagatcttccacgatgcacgcatgctttaaggttagatagtgtttggtccaaaaaagcgtcaacaatcaggaaattagaactaaaattattaaaggacagatcaaaaggcatgcatgttcttcttctatagtgtgtgttgagcctgagttttgattttaggctttattaggggactcgcagtctagctaaggagttgtattgatgttctgacaaatattatgttcgatcgtcacagtggtcttgtgcggatcgattaggcccgatcatggtgaaataaactaaccaccggtaagcccgggcagccctagagcatgcagcggcctacgtgaagcccgcgtgtcgcatcgtcgtccgtcagacgctaacggcaggccgctgcatgcgttgccggcgaactctctcctgagccactcgtcatccatataagtagacatcccatcactgtcgtctatcaacaacacacagagcgacatttcgaataacacagttgagcgcgaccatggacaagaagtacagcatcggcctggacatcggcaccaacagcgtgggctgggccgtgatcaccgacgagtacaaggtacgagcgggatacatgtttatactcctcctgtaggtcgctccttcatgtaatgtgttgcgattaaaacggtgcgcaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggacccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgagctcaattaactttgaattcccttcgattcatccggcgcggtgggctatggacctgcagcagcaagctaattaagtttatatatattgcatgagagagcatgcaccgctaaccatatatactactgagacttctgaattctagtatatgtaatccttttgtttgggtttaggaggcaattctaatcatgtatgccgaattccaaagagtggaaaacaagcaaaatgttaaatatacatgccattttcggaggcaatttttttcatgagggcatgttgctataattccggggaccttggacttcttggagcaccttcctgtgacttaggcatacatgattagattataatccaattagttaagtcatagaaaattacctcattctcatctccatctccatttctctatttcttctcaatcaaggaccaaaatagcacttttgctaaaaaacaagttagattgcaaaccaaagtgcacaatacatagtaaaaggtatatgcaacatatttgaatactcaaacctctcatacttacattttccatcattttgttccatttagcctgtttgagctcggggttggactccaaaacctcatgtcaacataacttgatccttttagcaaactatgagctctaacaccatacaatggtcaacaagaactattccaaacataggaatgacccaaactacaagtcaaagtatacttagctctttgggcacttacaggttctaactttgataattctgtacttcttgtgaccatgactctgctcgagctaggatcttgagccttatgacttaaacaattaaaccacaaacattacctcaatggttgtaagccacgtccatatatcacagacttcaatgcattcagactattcacagcttgaccaaccttgacctcttgcaagaacctcttcttctttgtgaccttaggtactttagtcttcttgaccttctcccttgctcttcataccttgaagtccttcttgccttcaccttagttcaatcagctatctccaagtcatgcacattgagttccacttagtcaatgtccatccttcaacttgacttgtgatgtccacaattcatagtcatctcagtctatgggtccatcatgcttgactccatgtgatgaaccttgtaaggttttcactaagtacatgctcagacctttaattgtgttgccatccaaaaaaaccaaaacctagattggaccattcattatattcatcaatcattgtacttgcaagagtgatcaaggtcatattatttctctcaactactccattttgttgaggggtgtcagttgtggagacttcttgtttgatcccaacctcatcacaatactcatgaatatagttgttgtcaaattcatttccattgtcacttcttatttttcttgattttgcaatcaaactcattttgtactttcatggtaaatttattcaatgttgatgcaacttttgacttttcttgaagaaagaacactcaattacatctagagaaatcatcaacaacgaccaaacaatacaggtttcccccaacactagcatattatgtaggaccaaataaatccatgtgaagtaactctagtggtcttggtgttgacataaaagcgtttgtaggatgtgtattggcaacttgttttccagcttgacatgcactataaaagattttcctttttcaaacacaacatctttcaaatctctaaccatttctttctttggaagcttcttgttggggaaatgatccccggaccctaggacccaccggtcagagagcgcgaggaagagcccccggtcgctgggacccgttggtccgctggaaaatgtggttacgtcaaccctgaaagaacccgcccctggttgagccccgtggcaccgagcctagggtcgagcgcggtggaatctgacaggaggggccagacatgttggaggggaaccactcaagtggatcccgcgcctggccccagaatgacccgtcattaatacccaaccacattaaccatgcctggcaccgagccatagcacggacgtcggtccacttcccactcatgacctacgaaccagttgggctgcatagcactcatgaccgataggttgaaggcttggcttcgcagagtgaaaggcgctgcatacatgtgaaggctcgacttctttttcttttcctttcttttcttttctatttttaggtttccaatttaaattccaatttttttgtggagttcatatttggatcaaatagacaaattcacctatcagtatgaatagatgcatttattttgtttatatctattttcttcatatttatatagtatttcccttattctttatatcattttcaatttgtaattggtaagtttggtcttaaattccccatttgggcactaatatatttttattaatattattattattattattattattatttatagatgcacaaacacataaactccgacatgatgcatagattattttagatgtcactagttaatggttcactttaaatatggttattcccatgttctaatgagtagagggcaaagcatatattgaggtcaactctttccttattatttacaaattggggaaattctattcataactcttcttctctctcccaagtagcttaatcttcaccatggtgatttcattgcactttgcacattttgatcactttattccttgtaacccgagtcaaagtgtcaatgatcttgataggatactccgtgcaggttagatcaccttgcacactgagttcttccattggtaactgttcctctggcggaccgttcgaagggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcagctggagctgagcttccggggttttagagctagaaatagcaagttaaaataaggctagtccgttatcaacttgaaaaagtggcaccgagtcggtgctttttttttcggaccgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccggcagcagccatgcagaagctgatcaacagcgtgcagaactacgcctggggcagcaagaccgccctgaccgagctgtacggcatggagaaccccagcagccagcccatggccgagctgtggatgggcgcccaccccaagagcagcagccgcgtgcagaacgccgccggcgacatcgtgagcctgcgcgacgtgatcgagagcgacaagagcaccctgctgggcgaggccgtggccaagcgcttcggcgagctgcccttcctgttcaaggtgctgtgcgccgcccagcccctgagcatccaggtgcaccccaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgtgagcctgctgcagcccgtggccggcgcccaccccgccatcgcccacttcctgcagcagcccgacgccgagcgcctgagcgagctgttcgccagcctgctgaacatgcagggcgaggagaagagccgcgccctggccatcctgaagagcgccctggacagccagcagggcgagccctggcagaccatccgcctgatcagcgagttctaccccgaggacagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggccaagcccgccaaccagctgctgacccagcccgtgaagcagggcgccgagctggacttccccatccccgtggacgacttcgccttcagcctgcacgacctgagcgacaaggagaccaccatcagccagcagagcgccgccatcctgttctgcgtggagggcgacgccaccctgtggaagggcagccagcagctgcagctgaagcccggcgagagcgccttcatcgccgccaacgagagccccgtgaccgtgaagggccacggccgcctggcccgcgtgtacaacaagctgtgataggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccgcaattgaagtttgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 36 - DNA, 17045 bp, synthetic construct
  misc_feature 1..17045: note = vector 24079
  misc_feature 4..259
  regulatory 312..2356: note = promoter - prGRMZM2G146551-01; regulatory_class = promoter
  gene 2358..6527: note = cCas9-01
  variation 5847..5849: note = mutation - L to V mutation
  variation 5892..5894: note = mutation - I to V mutation
  regulatory 6542..7860: note = terminator - tGRMZM2G146551-01; regulatory_class = terminator
  regulatory 7874..8248: note = promoter - prOsU3-01; regulatory_class = promoter
  misc_feature 8249..8354: note = rsgRNAZmVLHP-02
  misc_feature 8250..8269: note = ZmVLHP2 target
  misc_feature 8270..8281: note = rCrRNA-01
  misc_feature 8286..8354: note = rTracrRNA-01
  regulatory 8365..10356: note = promoter - prUbi1-04; regulatory_class = promoter
  gene 10373..11551: note = cPMI-09
  regulatory 11574..11826: note = terminator - tNOS-05-01; regulatory_class = terminator
  misc_feature 11870..11999: note = bNLB-03
  gene 12279..13067: note = cSpec-03
  regulatory 13162..13292: note = promoter - prVirG-01; regulatory_class = promoter
  gene 13367..13999: note = cVirG-09
  gene 14029..15102: note = cRepA-01
  misc_feature 15145..15549: note = oVS1-02
  misc_feature 16227..17033: note = oCOLE-06
  source 1..17045: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtaccgggacccatgtagtatcacatgagtgagtcaaggactaagtattatgcattttgtttctcactcacggattagctcgcaatcatcatagtgaaatctagctactggcactatcgaaatctagctctttgccgagtgcactttatcgagcactcgacaaagcattctttatcgagtgccagtcttggcgaaataagactctcgacaaagaccttgtttaccgagggagaaacactcggcgtaaaaagacactcggcaaagaagactttgctgagtgtcaaaccctcagcgaaatgcgaccctcggcaaaggaccgtcagcagccatctatagttgatggctattaacttcgcgagtgtcaggcgttgacacacgacaaaatatcttttttgtcgagtgtcactgggcaaacacttggtaaacctatgttttgccgagtgtctttccttgacactcgacaaagtatatttgttttttctttttccccaaactttttgtggtgtgtttctacaatatatagacctatttgttcaattttggcacaattataaaagtgtttgctataactatcagatttagtttgcttaattggatttctttggataattcagatttgaactacaagccacttgaaaaatggaaaacagtgaatacaaaaatgacattcatgttatttagcacaagttatgatctatttcaggaacatgcgagaattttcgaacaccattctcacaaaacatgattgcggacttgtgatcaagttgttttaaaattgtataaaacaaaaacaaagtcagaaaatcatgaaacttgttgacatgtcatgatatcatatgtagagactctaataaaaatttgagattgtttcatgaaagttgtcacgcgctatgtgtagaaacctagcccgtctacattgaggttctatgatttcatgtgaaggacatctaggcatcaatgtttatgataatatcttatgtttgtttggacgaaatattaaaaacaaataaaaaggggtccttgatcactttgacgagcattgcactcagcaaagggtgcctttgctgagtgcaatggtcatagaactcggtagaaaaacatacatagacatagggaaacttgctttaccgcgtgctatggccaagacactcggcaaactaggctcctttgtcgagttccatcccaagcactcgacattggaactgcgactgggcctcacagaagctttctttgccgagtgccactaagcgaggaactcggatgctcagcaaaggctctgtcatcgtcacgatgtcttttgtttgtcgtgtaccagttggcactcggtaaagactttactgagtgcccgatagaaagtactcgacaaagagaccgttgccaacgtttggttcactgagggctctttgctgccttttggacttgacaaagaagccgtctccagtagtgtctcctgggaggcgggatttatgttttttcccggagctctgtgggacatcatggacggtccagtctggtgatctaaaatagacggtttgccaagctcacagagaagtctttaagatcttccacgatgcacgcatgctttaaggttagttagtgtttggtctgaaaaagcgtcaacaattaggaaacaagaactaaaattattaaaggacagatcaggaagcatgcatgttcttcttctatagtgtgtgttgagcctgagtttggccttttaggctttattagggggctcacagtctaactaaggagttgtattgatgtgctgacaaatattatgttcgatcgtcacagtgttcttatgcggatcgattaggcccgatcatggtgaaataaactaaccaccggtaagcccgggcagccctagagcatgcagcggcctacgtgaagcccgcacatcgcatcgtcgtccgtcaggcgctaacggccggccgctgcatgcgtcgccggcgaactctctgctgagccacccgtcctccctataagtagctatcccagcaccgtcgtctatcaaccacacacagagcggcatttcgaataacacaggtgagcgcgaccatggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggacccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaccatggagctctaaactttgaattcccttcgattcatccggcacagcgggctatggaccttcagcagcaagctaattaagttggcagcatgcaccgctaaccttatatactactgagacttccaaattctagtatatgtaatccttttgttcgggttcatgatcgaattccaaagagtggaaaacaagcaaaaggttaaatatacatgccatttttggaggcatttttttcatgagggcatgtttcgatatatggaccactaaatatacatatcatttactttcctacaaatttgctacatccttggaaatgcatagtctgtctccaagaaaaagatactctgattacatcactagtacacacagcctctatagtggcggttctagagacattttcactggcgcttttcagtgccgccagtgttaggggccagtggaaatcgccatttccattcaataaccgccagtggaaaaagcatttccactggcggttttcttaagcaaccgccagtggaaatgtttcccgtctttttttaaattttcgtactgaaatttatatatttacacacacaaacatatatatatatatattgatattgataaacatgtagtattgatactaaaagcaacatgaaattaaattctatcatacatttatatacatcaaagtcttgtttacaaccatgtatgcatcacacattatatacatcaaagttttcacttaagctctaataactatctcggctaagagatagtctactaatttctgttagtattctaaactctggcaaagctaatgttccggaagcatcgtgatatttcccttctgcgggaatgacctctttcaatatgaatgtgcacaggtcctcaactatgccatacaatgcaccttcagtcaagttctccgggcttcctttttgaaattgctgtaaaggaagtttataaacatcatctatttatactcaataataacacatttgcatctttaatgacataaatacatacgtgactattactaataataccttgccagggttcgtgatgtatcgtccattcattctcataaactcgcacacgtagaacccacataggaccgatccgggtggttgcttgtggcactacataacgggagattggttatttagttgcaacattgtcctatgtacgtacatgtatgatatgtattcataaattcacatacttactggccagttataatggatgtctagtggcacaccttttttggacgtgtcgtactttccaccatgtagcttataaaacctaaatgccctgtgatctcaaatagaatcaccatgttattctacaattctcatgggacccttcgaagggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcagctggagctgagcttccggggttttagagctagaaatagcaagttaaaataaggctagtccgttatcaacttgaaaaagtggcaccgagtcggtgctttttttttcggaccgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccggcagcagccatgcagaagctgatcaacagcgtgcagaactacgcctggggcagcaagaccgccctgaccgagctgtacggcatggagaaccccagcagccagcccatggccgagctgtggatgggcgcccaccccaagagcagcagccgcgtgcagaacgccgccggcgacatcgtgagcctgcgcgacgtgatcgagagcgacaagagcaccctgctgggcgaggccgtggccaagcgcttcggcgagctgcccttcctgttcaaggtgctgtgcgccgcccagcccctgagcatccaggtgcaccccaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgtgagcctgctgcagcccgtggccggcgcccaccccgccatcgcccacttcctgcagcagcccgacgccgagcgcctgagcgagctgttcgccagcctgctgaacatgcagggcgaggagaagagccgcgccctggccatcctgaagagcgccctggacagccagcagggcgagccctggcagaccatccgcctgatcagcgagttctaccccgaggacagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggccaagcccgccaaccagctgctgacccagcccgtgaagcagggcgccgagctggacttccccatccccgtggacgacttcgccttcagcctgcacgacctgagcgacaaggagaccaccatcagccagcagagcgccgccatcctgttctgcgtggagggcgacgccaccctgtggaagggcagccagcagctgcagctgaagcccggcgagagcgccttcatcgccgccaacgagagccccgtgaccgtgaagggccacggccgcctggcccgcgtgtacaacaagctgtgataggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccgcaattgaagtttgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 37 - DNA, 16776 bp, synthetic construct
  misc_feature 1..16776: note = vector 24091
  misc_feature 4..259: note = bNRB-05
  regulatory 330..2417: note = promoter - prZmGRMZM2G471240-01; regulatory_class = promoter
  gene 2420..6589: note = cCas9-01
  variation 5909..5911: note = mutation - L to V mutation
  variation 5954..5956: note = mutation - I to V mutation
  regulatory 6596..7591: note = terminator - tZmGRMZM2G471240-01; regulatory_class = terminator
  regulatory 7605..7979: note = promoter - prOsU3-01; regulatory_class = promoter
  misc_feature 7981..8085: note = rsgRNAZmVLHP-02
  misc_feature 7981..8000: note = xZmVLHP2
  misc_feature 8001..8012: note = rCrRNA-01
  misc_feature 8017..8085: note = rTracrRNA-01
  regulatory 8096..10087: note = promoter - prUbi1-04; regulatory_class = promoter
  gene 10104..11282: note = cPMI-09
  regulatory 11305..11557: note = terminator - tNOS-05-01; regulatory_class = terminator
  misc_feature 11601..11730: note = bNLB-03
  gene 12010..12798: note = cSpec-03
  regulatory 12893..13023: note = promoter - prVirG-01; regulatory_class = promoter
  gene 13098..13730: note = cVirG-09
  gene 13760..14833: note = cRepA-01
  misc_feature 14876..15280: note = oVS1-02
  misc_feature 15958..16764: note = oCOLE-06
  source 1..16776: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtacctcgcgaatgcatctagatgggaccctatttgtactcattccatgtctcataaactttgggcaccatccatccaacacatccaatctaaacacaccaaacgatggggaatggaaagagcagtattcgattcaacaatggcaaacaaatatcactgaattagaccaagaataaacctaattagacaacgacctcccaaccatcattcgtcaggctgtaaagaagataaagctgccttggggcatggatcaagcagaacaccagagatgaatccaaacacacagaaaatcacgcgcgctgtctacaatgacaacaagccccacatttcattgcagtacactgggctacaaaggcacgtacaacaaagagctagggaaacattgcggagggcacgagagagcagctaacttgacaatatagcagactgagcttgcactgttagcaggcgaggaagggaatcatggggacggagaatggggtccatgcccgcgaaggagaaggcggacgccgccacggtggcaccggcgcacgcgcacacagggaacccgcacaggcagccaaggatgctgcctcgccattgcgccggtcgtctctgccacgctcctctctctctcccgctgcatcgccgtggatggggcaagcagagagcagggactgcgacgatctgggcggaggactcgccttggagagcgcggacgcagacgggattctagggagagagcgaagacggggcgcgcgcggcgctcgcgcggcgtggtggcggcgagattagcgggggtggggggagggcggagccgtggtgagggtgtggacgccctccttaccctcttaagtagtagtagagatataatccgttccaaaatatccatccgttcaatttatatttcgtttgatctttttaccctaaatttgattgactcatcttattaaaaaagttcataactattattaatctttattgagatatcatttagcatataatatactttaagtgtggttttagattttttttaaaaaaaaaaattcgcaaaaattaaatgaaacgacccaatcaaacttgaaaagtaaaactaattataaatttgaacggaaggagtaagaggatgtttgaatgtactagagctaatagttggttgctttaaaatttgctagtagaattagctagctaataaatatctagataactattagctaatttgctaaaacagctaatagttgaactattagctagattgtttggatgtattcggctaattttaatggctaactattagctatagtacaatattcaaacacctcctaattaaaatggacaaatatctcttcttttggtcccttgcgttagatttttcatatctccttatttagtataaaagaatcatcaaaaagtggacaacccctagtggaacaccattttagtagtggttgcatgaaacctttcgcgcaccagtttctatgtgtcactctaaaaatgggacagcatgtacgtagtgcctatatatatacaagtcatctatcgttgcctcctcagttcatcactaatcacacttattgtgccctcgacgagtatctatagctagctcattaatcgattcgggggtgtgttgtcgaaggcggcattggcgagctactcgtcgcggcgtccaagcaatacctgtagcacgaaggcgatcgccgggagcgtggtcggcgagcccgtcgtgctggggcagagggtgacggtgctgacggtggacggcggcggcgtccggggtctcatcccgggaaccatcctcgccttcctggaggccaggctgcaggagctggacgcaccggaggcgaggctggcggactacttcgactacatcgccggaaccagcaccggcggtctcatcaccgccttgctgaccgcgcccggcaaggacaagcggcctctctaggctgccaaggacatcaaccacttttacatccataactgcccgcgcatctttcctcagaagtgagtccgatgctgccgccattgttcttgcatccatccagcatcgtacgtacgtcctctatacatctgcggatcatcatgtgcgcatgtttgtggcatgcatgcatgcatgtgagcaggagcaggcttgcgaaaaccccatggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggacccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaccatgggacaagtggctttactgtcagtcacatgcttgtaaataagtagactttattttaataaaacataaaaatatatatatgttcttgaatataaaattgataaccaaattaaaattcgaaccatcacttatacataattttactttattttttataaaacgtgaacgggaaggactaccgtgaatgactatagaaccaatcatactagtataaaatatatgatgacactacgggagagacaaactttgtctggcgctaaatattttgccgagtgtgaattcacgggcactaggcaaagatcttctttgccgagtgttacgctgggcaaagtaagacactaggtaaatcagtcatttgccgagtgtccgccactaggcaaagcaaaacactggcaaatcaaaagtttacctagtgccagacactaggcaaaaaaaaaacgctcggcaaatcggaagtttccctagtgccagacactagacaaagaaaaacacttgataaactagcgtcgtcagctaacaccatccaccaaccgttaacgttgccgagtatctgacttcgacactcggcaaagaaggtctctttgcctagtgtcggtctggaacactaggcaaagaggcactttacctagtgtcgtattttgacactcagtaaaataattttttttctttctgcttccaaactttttatgatgtgttcctatagcacctagaactacatgtcaagttttggtaaaatttttgaagtttttgctatatttacttaatttattttatttaattgaatttcttttgataattcaaatttgaactcggcaaggtaagaagcgagggtagcctggaaacacactttgcctagtgttacactcggtacaggagcctcccctgcctagtgctgcactcgacaaaagattcgcctttgcctagcgctgcactcggcacaggagtcgcctttgcctagtgctgcactaggcaaagcctccgttaccgtgccttccatcgtcggacccttcgaagggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcagctggagctgagcttccggggttttagagctagaaatagcaagttaaaataaggctagtccgttatcaacttgaaaaagtggcaccgagtcggtgctttttttttcggaccgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccggcagcagccatgcagaagctgatcaacagcgtgcagaactacgcctggggcagcaagaccgccctgaccgagctgtacggcatggagaaccccagcagccagcccatggccgagctgtggatgggcgcccaccccaagagcagcagccgcgtgcagaacgccgccggcgacatcgtgagcctgcgcgacgtgatcgagagcgacaagagcaccctgctgggcgaggccgtggccaagcgcttcggcgagctgcccttcctgttcaaggtgctgtgcgccgcccagcccctgagcatccaggtgcaccccaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgtgagcctgctgcagcccgtggccggcgcccaccccgccatcgcccacttcctgcagcagcccgacgccgagcgcctgagcgagctgttcgccagcctgctgaacatgcagggcgaggagaagagccgcgccctggccatcctgaagagcgccctggacagccagcagggcgagccctggcagaccatccgcctgatcagcgagttctaccccgaggacagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggccaagcccgccaaccagctgctgacccagcccgtgaagcagggcgccgagctggacttccccatccccgtggacgacttcgccttcagcctgcacgacctgagcgacaaggagaccaccatcagccagcagagcgccgccatcctgttctgcgtggagggcgacgccaccctgtggaagggcagccagcagctgcagctgaagcccggcgagagcgccttcatcgccgccaacgagagccccgtgaccgtgaagggccacggccgcctggcccgcgtgtacaacaagctgtgataggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccgcaattgaagtttgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 38 - DNA, 17475 bp, synthetic construct
  misc_feature 1..17475: note = vector 24094
  misc_feature 4..259: note = bNRB-05
  regulatory 330..2417: note = promoter - prZmGRMZM2G471240-01; regulatory_class = promoter
  gene 2420..7288: note = cAmCyanCas9-01
  regulatory 7295..8290: note = terminator - tZmGRMZM2G471240-01; regulatory_class = terminator
  regulatory 8304..8678: note = promoter - prOsU3-01; regulatory_class = promoter
  misc_feature 8679..8784: note = rsgRNAZmVLHP-02
  misc_feature 8680..8699: note = ZmVLHP2 target
  misc_feature 8700..8711: note = rCrRNA-01
  misc_feature 8716..8784: note = rTracrRNA-01
  regulatory 8795..10786: note = promoter - prUbi1-04; regulatory_class = promoter
  gene 10803..11981: note = cPMI-09
  regulatory 12004..12256: note = terminator - tNOS-05-01; regulatory_class = terminator
  misc_feature 12300..12429: note = bNLB-03
  gene 12709..13497: note = cSpec-03
  regulatory 13592..13722: note = promoter - prVirG-01; regulatory_class = promoter
  gene 13797..14429: note = cVirG-09
  gene 14459..15532: note = cRepA-01
  misc_feature 15575..15979: note = oVS1-02
  misc_feature 16657..17463: note = oCOLE-06
  source 1..17475: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtacctcgcgaatgcatctagatgggaccctatttgtactcattccatgtctcataaactttgggcaccatccatccaacacatccaatctaaacacaccaaacgatggggaatggaaagagcagtattcgattcaacaatggcaaacaaatatcactgaattagaccaagaataaacctaattagacaacgacctcccaaccatcattcgtcaggctgtaaagaagataaagctgccttggggcatggatcaagcagaacaccagagatgaatccaaacacacagaaaatcacgcgcgctgtctacaatgacaacaagccccacatttcattgcagtacactgggctacaaaggcacgtacaacaaagagctagggaaacattgcggagggcacgagagagcagctaacttgacaatatagcagactgagcttgcactgttagcaggcgaggaagggaatcatggggacggagaatggggtccatgcccgcgaaggagaaggcggacgccgccacggtggcaccggcgcacgcgcacacagggaacccgcacaggcagccaaggatgctgcctcgccattgcgccggtcgtctctgccacgctcctctctctctcccgctgcatcgccgtggatggggcaagcagagagcagggactgcgacgatctgggcggaggactcgccttggagagcgcggacgcagacgggattctagggagagagcgaagacggggcgcgcgcggcgctcgcgcggcgtggtggcggcgagattagcgggggtggggggagggcggagccgtggtgagggtgtggacgccctccttaccctcttaagtagtagtagagatataatccgttccaaaatatccatccgttcaatttatatttcgtttgatctttttaccctaaatttgattgactcatcttattaaaaaagttcataactattattaatctttattgagatatcatttagcatataatatactttaagtgtggttttagattttttttaaaaaaaaaaattcgcaaaaattaaatgaaacgacccaatcaaacttgaaaagtaaaactaattataaatttgaacggaaggagtaagaggatgtttgaatgtactagagctaatagttggttgctttaaaatttgctagtagaattagctagctaataaatatctagataactattagctaatttgctaaaacagctaatagttgaactattagctagattgtttggatgtattcggctaattttaatggctaactattagctatagtacaatattcaaacacctcctaattaaaatggacaaatatctcttcttttggtcccttgcgttagatttttcatatctccttatttagtataaaagaatcatcaaaaagtggacaacccctagtggaacaccattttagtagtggttgcatgaaacctttcgcgcaccagtttctatgtgtcactctaaaaatgggacagcatgtacgtagtgcctatatatatacaagtcatctatcgttgcctcctcagttcatcactaatcacacttattgtgccctcgacgagtatctatagctagctcattaatcgattcgggggtgtgttgtcgaaggcggcattggcgagctactcgtcgcggcgtccaagcaatacctgtagcacgaaggcgatcgccgggagcgtggtcggcgagcccgtcgtgctggggcagagggtgacggtgctgacggtggacggcggcggcgtccggggtctcatcccgggaaccatcctcgccttcctggaggccaggctgcaggagctggacgcaccggaggcgaggctggcggactacttcgactacatcgccggaaccagcaccggcggtctcatcaccgccttgctgaccgcgcccggcaaggacaagcggcctctctaggctgccaaggacatcaaccacttttacatccataactgcccgcgcatctttcctcagaagtgagtccgatgctgccgccattgttcttgcatccatccagcatcgtacgtacgtcctctatacatctgcggatcatcatgtgcgcatgtttgtggcatgcatgcatgcatgtgagcaggagcaggcttgcgaaaaccccatggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttcggcggcggcggatccgacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggacccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaccatgggacaagtggctttactgtcagtcacatgcttgtaaataagtagactttattttaataaaacataaaaatatatatatgttcttgaatataaaattgataaccaaattaaaattcgaaccatcacttatacataattttactttattttttataaaacgtgaacgggaaggactaccgtgaatgactatagaaccaatcatactagtataaaatatatgatgacactacgggagagacaaactttgtctggcgctaaatattttgccgagtgtgaattcacgggcactaggcaaagatcttctttgccgagtgttacgctgggcaaagtaagacactaggtaaatcagtcatttgccgagtgtccgccactaggcaaagcaaaacactggcaaatcaaaagtttacctagtgccagacactaggcaaaaaaaaaacgctcggcaaatcggaagtttccctagtgccagacactagacaaagaaaaacacttgataaactagcgtcgtcagctaacaccatccaccaaccgttaacgttgccgagtatctgacttcgacactcggcaaagaaggtctctttgcctagtgtcggtctggaacactaggcaaagaggcactttacctagtgtcgtattttgacactcagtaaaataattttttttctttctgcttccaaactttttatgatgtgttcctatagcacctagaactacatgtcaagttttggtaaaatttttgaagtttttgctatatttacttaatttattttatttaattgaatttcttttgataattcaaatttgaactcggcaaggtaagaagcgagggtagcctggaaacacactttgcctagtgttacactcggtacaggagcctcccctgcctagtgctgcactcgacaaaagattcgcctttgcctagcgctgcactcggcacaggagtcgcctttgcctagtgctgcactaggcaaagcctccgttaccgtgccttccatcgtcggacccttcgaagggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcagctggagctgagcttccggggttttagagctagaaatagcaagttaaaataaggctagtccgttatcaacttgaaaaagtggcaccgagtcggtgctttttttttcggaccgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccggcagcagccatgcagaagctgatcaacagcgtgcagaactacgcctggggcagcaagaccgccctgaccgagctgtacggcatggagaaccccagcagccagcccatggccgagctgtggatgggcgcccaccccaagagcagcagccgcgtgcagaacgccgccggcgacatcgtgagcctgcgcgacgtgatcgagagcgacaagagcaccctgctgggcgaggccgtggccaagcgcttcggcgagctgcccttcctgttcaaggtgctgtgcgccgcccagcccctgagcatccaggtgcaccccaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgtgagcctgctgcagcccgtggccggcgcccaccccgccatcgcccacttcctgcagcagcccgacgccgagcgcctgagcgagctgttcgccagcctgctgaacatgcagggcgaggagaagagccgcgccctggccatcctgaagagcgccctggacagccagcagggcgagccctggcagaccatccgcctgatcagcgagttctaccccgaggacagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggccaagcccgccaaccagctgctgacccagcccgtgaagcagggcgccgagctggacttccccatccccgtggacgacttcgccttcagcctgcacgacctgagcgacaaggagaccaccatcagccagcagagcgccgccatcctgttctgcgtggagggcgacgccaccctgtggaagggcagccagcagctgcagctgaagcccggcgagagcgccttcatcgccgccaacgagagccccgtgaccgtgaagggccacggccgcctggcccgcgtgtacaacaagctgtgataggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccgcaattgaagtttgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 39 - DNA, 19 bp, synthetic construct
  misc_feature 1..19: note = primer
  source 1..19: mol_type = other DNA
ttgtgctgctccacgaaca

SEQ ID NO: 40 - DNA, 20 bp, synthetic construct
  misc_feature 1..20: note = primer
  source 1..20: mol_type = other DNA
gccagccactacgagaagct

SEQ ID NO: 41 - DNA, 24 bp, synthetic construct
  misc_feature 1..24: note = probe
  source 1..24: mol_type = other DNA
ctgcttctgctcgttgtcctccgg

SEQ ID NO: 42 - DNA, 17 bp, synthetic construct
  misc_feature 1..17: note = promer
  source 1..17: mol_type = other DNA
gcggatgctggcacagc

SEQ ID NO: 43 - DNA, 19 bp, synthetic construct
  misc_feature 1..19: note = primer
  source 1..19: mol_type = other DNA
ggcattgcttccttctccg

SEQ ID NO: 44 - DNA, 15 bp, synthetic construct
  misc_feature 1..15: note = probe
  source 1..15: mol_type = other DNA
cagggagcgaggtac

SEQ ID NO: 45 - DNA, 20 bp, synthetic construct
  misc_feature 1..20: note = primer
  source 1..20: mol_type = other DNA
ctggtggccaacgtgaagtt

SEQ ID NO: 46 - DNA, 17 bp, synthetic construct
  misc_feature 1..17: note = primer
  source 1..17: mol_type = other DNA
gcttcacgggctgggtc

SEQ ID NO: 47 - DNA, 20 bp, synthetic construct
  misc_feature 1..20: note = probe
  source 1..20: mol_type = other DNA
aggccaagcccgccaaccag

SEQ ID NO: 48 - DNA, 17 bp, synthetic construct
  misc_feature 1..17: note = primer
  source 1..17: mol_type = other DNA
gcggatgctggcacaga

SEQ ID NO: 49 - DNA, 18 bp, synthetic construct
  misc_feature 1..18: note = primer
  source 1..18: mol_type = other DNA
gcattgcttccttcgcca

SEQ ID NO: 50 - DNA, 16 bp, synthetic construct
  misc_feature 1..16: note = probe
  source 1..16: mol_type = other DNA
cagggaggtacgaacc

SEQ ID NO: 51 - DNA, 16 bp, synthetic construct
  misc_feature 1..16: note = primer
  source 1..16: mol_type = other DNA
gcggcgaagaagcgaa

SEQ ID NO: 52 - DNA, 17 bp, synthetic construct
  misc_feature 1..17: note = primer
  source 1..17: mol_type = other DNA
gcggcgtctccagcttc

SEQ ID NO: 53 - DNA, 12 bp, synthetic construct
  misc_feature 1..12: note = probe
  source 1..12: mol_type = other DNA
ccaggaactgcg

SEQ ID NO: 54 - DNA, 18 bp, synthetic construct
  misc_feature 1..18: note = primer
  source 1..18: mol_type = other DNA
aagaaacgccggctgagt

SEQ ID NO: 55 - DNA, 15 bp, synthetic construct
  misc_feature 1..15: note = primer
  source 1..15: mol_type = other DNA
accttgcggggcgtt

SEQ ID NO: 56 - DNA, 12 bp, synthetic construct
  misc_feature 1..12: note = probe
  source 1..12: mol_type = other DNA
ccaggaactgcg

SEQ ID NO: 57 - DNA, 18 bp, synthetic construct
  misc_feature 1..18: note = primer
  source 1..18: mol_type = other DNA
aagaaacgccggctgagt

SEQ ID NO: 58 - DNA, 14 bp, synthetic construct
  misc_feature 1..14: note = primer
  source 1..14: mol_type = other DNA
ccttgcgcggcgtc

SEQ ID NO: 59 - DNA, 12 bp, synthetic construct
  misc_feature 1..12: note = probe
  source 1..12: mol_type = other DNA
ccaggaactgcg

SEQ ID NO: 60 - DNA, 19 bp, synthetic construct
  misc_feature 1..19: note = primer
  source 1..19: mol_type = other DNA
tgatcctcgaggccaagct

SEQ ID NO: 61 - DNA, 18 bp, synthetic construct
  misc_feature 1..18: note = primer
  source 1..18: mol_type = other DNA
aggtcgaggtcccctcca

SEQ ID NO: 62 - DNA, 14 bp, synthetic construct
  misc_feature 1..14: note = probe
  source 1..14: mol_type = other DNA
cctgctacccgggc

SEQ ID NO: 63 - DNA, 15 bp, synthetic construct
  misc_feature 1..15: note = primer
  source 1..15: mol_type = other DNA
cgcgccctgctaccc

SEQ ID NO: 64 - DNA, 17 bp, synthetic construct
  misc_feature 1..17: note = primer
  source 1..17: mol_type = other DNA
gcgcgtgcttaccagga

SEQ ID NO: 65 - DNA, 13 bp, synthetic construct
  misc_feature 1..13: note = probe
  source 1..13: mol_type = other DNA
tcgaggagtgccc

SEQ ID NO: 66 - DNA, 16 bp, synthetic construct
  misc_feature 1..16: note = primer
  source 1..16: mol_type = other DNA
caccgatgagcaggcg

SEQ ID NO: 67 - DNA, 18 bp, synthetic construct
  misc_feature 1..18: note = primer
  source 1..18: mol_type = other DNA
agatacaccttccggccg

SEQ ID NO: 68 - DNA, 14 bp, synthetic construct
  misc_feature 1..14: note = probe
  source 1..14: mol_type = other DNA
ttcctcccggaagc

SEQ ID NO: 69 - DNA, 16 bp, synthetic construct
  misc_feature 1..16: note = primer
  source 1..16: mol_type = other DNA
caccgatgagcaggcg

SEQ ID NO: 70 - DNA, 20 bp, synthetic construct
  misc_feature 1..20: note = primer
  source 1..20: mol_type = other DNA
agatacaccttccggccagt

SEQ ID NO: 71 - DNA, 14 bp, synthetic construct
  misc_feature 1..14: note = probe
  source 1..14: mol_type = other DNA
ctcctcccggaagc

SEQ ID NO: 72 - DNA, 25 bp, synthetic construct
  misc_feature 1..25: note = primer
  source 1..25: mol_type = other DNA
caagtttctggacaaggagattctc

SEQ ID NO: 73 - DNA, 27 bp, synthetic construct
  misc_feature 1..27: note = primer
  source 1..27: mol_type = other DNA
aagaattcccttcttaatagctggaga

SEQ ID NO: 74 - DNA, 26 bp, synthetic construct
  misc_feature 1..26: note = probe
  source 1..26: mol_type = other DNA
cacgagcacattgctaaccttgctgg

SEQ ID NO: 75 - DNA, 17 bp, synthetic construct
  misc_feature 1..17: note = primer
  source 1..17: mol_type = other DNA
tcaccgatgagcaggca

SEQ ID NO: 76 - DNA, 18 bp, synthetic construct
  misc_feature 1..18: note = primer
  source 1..18: mol_type = other DNA
atacaccttccggccagc

SEQ ID NO: 77 - DNA, 14 bp, synthetic construct
  misc_feature 1..14: note = probe
  source 1..14: mol_type = other DNA
ttcctcccggaagc

SEQ ID NO: 78 - DNA, 24 bp, synthetic construct
  misc_feature 1..24: note = primer
  source 1..24: mol_type = other DNA
gatagggctaaagagatgtgggaa

SEQ ID NO: 79 - DNA, 26 bp, synthetic construct
  misc_feature 1..26: note = primer
  source 1..26: mol_type = other DNA
ctttgttcacattagggctcaaataa

SEQ ID NO: 80 - DNA, 16 bp, synthetic construct
  misc_feature 1..16: note = probe
  source 1..16: mol_type = other DNA
tagactgagatggatg

SEQ ID NO: 81 - DNA, 20 bp, synthetic construct
  misc_feature 1..20: note = primer
  source 1..20: mol_type = other DNA
aaaaccaccggagaagacga

SEQ ID NO: 82 - DNA, 17 bp, synthetic construct
  misc_feature 1..17: note = primer
  source 1..17: mol_type = other DNA
aggtgtggcggcagtga

SEQ ID NO: 83 - DNA, 14 bp, synthetic construct
  misc_feature 1..14: note = probe
  source 1..14: mol_type = other DNA
caccgtcattgttc

SEQ ID NO: 84 - DNA, 25 bp, synthetic construct
  misc_feature 1..25: note = primer
  source 1..25: mol_type = other DNA
caagtttctggacaaggagattctc

SEQ ID NO: 85 - DNA, 27 bp, synthetic construct
  misc_feature 1..27: note = primer
  source 1..27: mol_type = other DNA
aagaattcccttcttaatagctggaga

SEQ ID NO: 86 - DNA, 26 bp, synthetic construct
  misc_feature 1..26: note = probe
  source 1..26: mol_type = other DNA
cacgagcacattgctaaccttgctgg

SEQ ID NO: 87 - DNA, 15 bp, synthetic construct
  misc_feature 1..15: note = primer
  source 1..15: mol_type = other DNA
gcgacgccggaaagg

SEQ ID NO: 88 - DNA, 21 bp, synthetic construct
  misc_feature 1..21: note = primer
  source 1..21: mol_type = other DNA
tggcgtggtttcgtcttctta

SEQ ID NO: 89 - DNA, 25 bp, synthetic construct
  misc_feature 1..25: note = probe
  source 1..25: mol_type = other DNA
aagagcggcgtctggaggtgactca

SEQ ID NO: 90 - DNA, 20 bp, synthetic construct
  misc_feature 1..20: note = primer
  source 1..20: mol_type = other DNA
aaccgcatcgtcagaaaaac

SEQ ID NO: 91 - DNA, 20 bp, synthetic construct
  misc_feature 1..20: note = primer
  source 1..20: mol_type = other DNA
tcaacttaaccggccaaatc

SEQ ID NO: 92 - DNA, 21 bp, synthetic construct
  misc_feature 1..21: note = primer
  source 1..21: mol_type = other DNA
catcccttctcttccctcctg

SEQ ID NO: 93 - DNA, 23 bp, synthetic construct
  misc_feature 1..23: note = primer
  source 1..23: mol_type = other DNA
gccagtgtgagtgtgtatgagca

SEQ ID NO: 94 - DNA, 21 bp, synthetic construct
  misc_feature 1..21: note = primer
  source 1..21: mol_type = other DNA
catcgttttctcccctcctca

SEQ ID NO: 95 - DNA, 19 bp, synthetic construct
  misc_feature 1..19: note = primer
  source 1..19: mol_type = other DNA
actgatatgcacggcgcca

SEQ ID NO: 96 - DNA, 22 bp, synthetic construct
  misc_feature 1..22: note = primer
  source 1..22: mol_type = other DNA
tgcagtagcttcattttcaccg

SEQ ID NO: 97 - DNA, 22 bp, synthetic construct
  misc_feature 1..22: note = primer
  source 1..22: mol_type = other DNA
aggaattgatatgtacgcccgt

SEQ ID NO: 98 - DNA, 16279 bp, synthetic construct
  misc_feature 1..16279: note = vector 24075
  misc_feature 1..517: note = bNRB-07
  regulatory 538..1697: note = promoter - prAtEFaA1-02; regulatory_class = promoter
  gene 1716..5885: note = cCas9-05
  variation 5205..5207: note = mutation - L to V mutation
  variation 5250..5252: note = mutation - I to V mutation
  regulatory 5894..6146: note = terminator - tNOS-05-01; regulatory_class = terminator
  regulatory 6173..6620: note = promoter - prAtU6-01; regulatory_class = promoter
  misc_feature 6621..6640: note = AtGL1 target1
  misc_feature 6621..6725: note = rsgRNA AtGL1-01
  misc_feature 6641..6652: note = rCrRNA-01
  misc_feature 6657..6725: note = rTracrRNA-01
  regulatory 6726..7173: note = promoter - prAtU6-01; regulatory_class = promoter
  misc_feature 7174..7193: note = AtGl1 target 2
  misc_feature 7174..7278: note = rsgRNA AtGL1-02
  misc_feature 7194..7205: note = rCrRNA-01
  misc_feature 7210..7278: note = rTracrRNA-01
  regulatory 7295..7640: note = promoter - prCMP-02; regulatory_class = promoter
  gene 7653..8447: note = cNpt2-10
  regulatory 8476..8728: note = terminator - tNOS-05-01; regulatory_class = terminator
  regulatory 8755..10752: note = promoter - prGmUBI-01; regulatory_class = promoter
  gene 10765..11454: note = cAmCyan-06
  regulatory 11477..12119: note = terminator - tPsE9-01; regulatory_class = terminator
  misc_feature 12193..12311: note = bNLB-03
  gene 12928..13716: note = cSpec-03
  gene 13751..14824: note = cRepA-08
  misc_feature 14867..15271: note = oVC1-04
  misc_feature 15441..16247: note = oCOLE-06
  source 1..16279: mol_type = other DNA
gccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggcgcgccactcagcaagcttgatatcggaagtttctctcttgagggaggttgctcgtggaatgggacacatatggttgttataataaaccatttccattgtcatgagattttgaggttaatatatactttacttgttcattattttatttggtgtttgaataaatgatataaatggctcttgataatctgcattcattgagatatcaaatatttactctagagaagagtgtcatatagattgatggtccacaatcaatgaaatttttgggagacgaacatgtataaccatttgcttgaataaccttaattaaaaggtgtgattaaatgatgtttgtaacatgtagtactaaacattcataaaacacaaccaacccaagaggtattgagtattcacggctaaacaggggcataatggtaatttaaagaatgatattattttatgttaaaccctaacattggtttcggattcaacgctataaataaaaccactctcgttgctgattccatttatcgttcttattgaccctagccgctacacacttttctgcgatatctctgaggtaagcgttaacgtacccttagatcgttctttttctttttcgtctgctgatcgttgctcatattatttcgatgattgttggattcgatgctctttgttgattgatcgttctgaaaattctgatctgttgtttagattttatcgattgttaatatcaacgtttcactgcttctaaacgataatttattcatgaaactattttcccattctgatcgatcttgttttgagattttaatttgttcgattgattgttggttggtggatctatatacgagtgaacttgttgatttgcgtatttaagatgtatgtcgatttgaattgtgattgggtaattctggagtagcataacaaatccagtgttccctttttctaagggtaattctcggattgtttgctttatatctcttgaaattgccgatttgattgaatttagctcgcttagctcagatgatagagcaccacaatttttgtggtagaaatcggtttgactccgatagcggctttttactatgattgttttgtgttaaagatgattttcataatggttatatatgtctactgtttttattgattcaatatttgattgttcttttttttgcagatttgttgaccagggatccgcggccgctaaaatggataagaagtattctattggacttgatattggaaccaactctgtgggatgggctgttattactgacgagtataaggttccatctaagaagttcaaggttcttggaaacactgatagacactctattaagaagaaccttattggtgctcttcttttcgattctggagagactgctgaggctactagacttaagagaactgctagaagaagatatactagaagaaagaacagaatttgctatcttcaagagattttctctaacgagatggctaaggttgacgattctttcttccacagacttgaggagtctttccttgttgaggaggataagaagcacgagagacacccaattttcggaaacattgttgacgaggttgcttatcacgagaagtatccaactatttatcaccttagaaagaagctcgttgattctactgataaggctgatcttagacttatttatcttgctcttgctcacatgattaagttcagaggacacttccttattgagggagatcttaacccagataactctgacgttgataagctcttcattcaacttgttcaaacttataaccaacttttcgaggagaacccaattaacgcttctggagttgacgctaaggctattctttctgctagactttctaagtctagaaggcttgagaaccttattgctcaacttccaggagagaagaagaacggacttttcggaaaccttattgctctttctcttggacttactccaaacttcaagtctaacttcgatcttgctgaggacgctaagctccaactttctaaggatacttacgacgatgatcttgataaccttcttgctcaaattggagatcaatacgctgatcttttccttgctgctaagaacctttctgacgctattcttctttctgatattcttagagttaacactgagattactaaggctccactttctgcttctatgattaagagatacgacgagcaccaccaagatcttactcttcttaaggctcttgttagacaacaacttccagagaagtataaggagattttcttcgatcaatctaagaacggatacgctggatatattgacggaggagcttctcaagaggagttctataagttcattaagccaattcttgagaagatggacggaactgaggagcttcttgttaagctcaacagagaggatcttcttagaaagcaaagaactttcgataacggatctattccacaccaaattcaccttggagagcttcacgctattcttagaaggcaagaggatttctatccattccttaaggataacagagagaagattgagaagattcttactttccgtattccatattacgttggaccacttgctagaggaaactctagattcgcttggatgactagaaagtctgaggagactattactccttggaacttcgaggaggttgttgataagggagcttctgctcaatctttcattgagagaatgactaacttcgataagaaccttccaaacgagaaggttcttccaaagcactctcttctttacgagtatttcactgtttataacgagcttactaaggttaagtacgttactgagggaatgagaaagccagctttcctttctggagagcaaaagaaggctattgttgatcttcttttcaagactaacagaaaggttactgttaagcaacttaaggaggattatttcaagaagattgagtgcttcgattctgttgagatttctggagttgaggatagattcaacgcttctcttggaacttatcacgatcttcttaagattattaaggataaggatttccttgataacgaggagaacgaggatattcttgaggatattgttcttactcttactcttttcgaggatagagagatgattgaggagagacttaagacttacgctcaccttttcgacgataaggttatgaagcaacttaagagaagaagatatactggatggggtagactttctagaaagctcattaacggaattagagataagcaatctggaaagactattcttgatttccttaagtctgacggattcgctaacagaaacttcatgcaacttattcacgacgattctcttactttcaaggaggatattcaaaaggctcaagtttctggacaaggagattctcttcacgagcacattgctaaccttgctggatctccagctattaagaagggaattcttcaaactgttaaggttgttgacgagcttgttaaggttatgggtagacacaagccagagaacattgttattgagatggctagagagaaccaaactactcaaaagggacaaaagaactctagagagagaatgaagagaattgaggagggaattaaggagcttggatctcaaattcttaaggagcacccagttgagaacactcaacttcaaaacgagaagctctatctttattatcttcaaaacggaagagatatgtacgttgatcaagagcttgatattaacagactttctgattacgacgttgatcacattgttccacaatctttccttaaggacgattctattgataacaaggttcttactagatctgataagaacagaggaaagtctgataacgttccatctgaggaggttgttaagaagatgaagaactattggagacaacttcttaacgctaagctcattactcaaagaaagttcgataaccttactaaggctgagagaggaggactttctgagcttgataaggctggattcattaagagacaacttgttgagactagacaaattactaagcacgttgctcaaattcttgattctagaatgaacactaagtacgacgagaacgataagctcattagagaggttaaggttattactcttaagtctaagctcgtttctgatttcagaaaggatttccaattctataaggttagagagattaacaactatcaccacgctcacgacgcttatcttaacgctgttgttggaactgctcttattaagaagtatccaaaacttgagtctgagttcgtttacggagattataaggtttacgacgttagaaagatgattgctaagtctgagcaagagattggaaaggctactgctaagtatttcttctattctaacattatgaacttcttcaagactgagattactcttgctaacggagagattagaaagaggccacttattgagactaacggagagactggagagattgtttgggataagggaagagatttcgctactgttagaaaggttctttctatgccacaagttaacattgttaagaaaactgaggttcaaactggaggattctctaaggagtctattcttccaaagagaaactctgataagctcattgctagaaagaaggattgggacccaaagaagtacggaggattcgattctccaactgttgcttattctgttcttgttgttgctaaggttgagaagggaaagtctaagaagctcaagtctgttaaggagcttgttggaattactattatggagagatcttctttcgagaagaacccagttgatttccttgaggctaagggatataaggaggttaagaaggatcttattattaagctcccaaagtattctcttttcgagcttgagaacggaagaaagagaatgcttgcttctgctggagagcttcaaaagggaaacgagcttgctcttccatctaagtacgttaacttcctttatcttgcttctcactacgagaagctcaagggatctccagaggataacgagcaaaagcaacttttcgttgagcaacacaagcactatcttgacgagattattgagcaaatttctgagttctctaagagagttattcttgctgacgctaaccttgataaggttctttctgcttataacaagcacagagataagccaattagagagcaagctgagaacattattcaccttttcactcttactaaccttggtgctccagctgctttcaagtatttcgatactactattgatagaaagagatatacttctactaaggaggttcttgacgctactcttattcaccaatctattactggactttacgagactagaattgatctttctcaacttggaggagattcttctccaccaaagaagaagagaaaggtttcttggaaggacgcttctggatggtctagaatgtgacgtcgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgcagatcggacccctaattagctaaaagcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattggaaaagttgtagactgagagttttagagctagaaatagcaagttaaaataaggctagtccgttatcaacttgaaaaagtggcaccgagtcggtgctttttttttaagcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgcagtgatgaacaatgacgggttttagagctagaaatagcaagttaaaataaggctagtccgttatcaacttgaaaaagtggcaccgagtcggtgctttttttttggcgcgcctaaagcttctggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaggatcctaaaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatgagagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtaccctaattagctaaattccaaaattttcagttagtccttactaattattaaattatagtattaatccaatgtgattgcggttacatcatgtacggaaaaataattctaatccttgatttaaatttgatcttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgctttgataaaattaattggaaagtataaaatggtagaaaataataaattataattaatttaaataagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggcacgcgtcacaatatgattggagaacggttccaccgcatatgctataaaatgcccccacacccctcgaccctaatcgcacttcaattgcaatcaaattagttcattctctttgcgcagttccctacctctcctttcaaggttcgtagatttcttctgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacagagatctaaaaaaatggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgacgatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggatccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctgatgaactagtgaattcgagctcaagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcggaccgttaactagctagacggccaggatcgccgcgtgagcctttagcaactagctagattaattaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgtcgactcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtaccaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggacacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggccattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggacaaacaaacaaatacagtaattta

SEQ ID NO: 99 - DNA, 100 bp, Zea mays
  source 1..100: mol_type = genomic DNA
aagctgcgcaagctcatcctcgaggccaagctcgcgccctgctacccgggcgccgacgacgccgcgcccggcggaggggacctcgaggagtgccccatct

SEQ ID NO: 100 - DNA, 100 bp, Zea mays
  source 1..100: mol_type = genomic DNA
agatggggcactcctcgaggtcccctccgccgggcgcggcgtcgtcggcgcccgggtagcagggcgcgagcttggcctcgaggatgagcttgcgcagctt

SEQ ID NO: 101 - DNA, 420 bp, Triticum aestivum
  misc_feature 15: note = n is a, c, g, or t
  source 1..420: mol_type = genomic DNA
ctcatcgagtgttcnccgcaatgcgctgttgctgattctcaagtgcgtgtgggtgcaggtggagagcagaagaaggccggccggcagcggcggaggaggagggcgaggcaggcagcggcaggcgaaggcgcgggaggggacgatgcggcgaagaaacgccggctgagtgacgagcaggcgcagttcctggagatgagcttcaggaaggaacgtaaactggaaacgccccgcaaggtgcagctcgccgcggagctgggcctggacaccaagcaggtcgcggtgtggttccagaaccgccgcgcccgctacaagagcaagctcatcgaggaggagttctccaagctccgcgcggcacacgacgccgtcgtcgtccacaactgccacctcgaggccgaggtacagtgcaacagtccggctgcc

SEQ ID NO: 102 - DNA, 23 bp, synthetic construct
  misc_feature 1..23: note = gRNA target sequence
  source 1..23: mol_type = other DNA
ggaaaagttgtagactgagatgg

SEQ ID NO: 103 - DNA, 85 bp, Arabidopsis thaliana
  source 1..85: mol_type = genomic DNA
taaatttgattcgttgatagggctaaagagatgtgggaaaagttgtagactgagatggatgaattatttgagccctaatgtgaac

SEQ ID NO: 104 - DNA, 84 bp, Arabidopsis thaliana
  source 1..84: mol_type = genomic DNA
taaatttgattcgttgatagggctaaagagatgtgggaaaagttgtagactagatggatgaattatttgagccctaatgtgaac

SEQ ID NO: 105 - DNA, 86 bp, Arabidopsis thaliana
  source 1..86: mol_type = genomic DNA
taaatttgattcgttgatagggctaaagagatgtgggaaaagttgtagactgtagatggatgaattatttgagccctaatgtgaac

SEQ ID NO: 106 - DNA, 85 bp, Arabidopsis thaliana; identical to SEQ ID NO: 103
  source 1..85: mol_type = genomic DNA

SEQ ID NO: 107 - DNA, 86 bp, Arabidopsis thaliana
  source 1..86: mol_type = genomic DNA
taaatttgattcgttgatagggctaaagagatgtgggaaaagttgtagactgaagatggatgaattatttgagccctaatgtgaac

SEQ ID NO: 108 - DNA, 86 bp, Arabidopsis thaliana; identical to SEQ ID NO: 105
  source 1..86: mol_type = genomic DNA

SEQ ID NO: 109 - DNA, 82 bp, Arabidopsis thaliana
  source 1..82: mol_type = genomic DNA
taaatttgattcgttgatagggctaaagagatgtgggaaaagttgtagaagatggatgaattatttgagccctaatgtgaac

SEQ ID NO: 110 - DNA, 86 bp, Arabidopsis thaliana; identical to SEQ ID NO: 107
  source 1..86: mol_type = genomic DNA

SEQ ID NO: 111 - DNA, 84 bp, Arabidopsis thaliana; identical to SEQ ID NO: 104
  source 1..84: mol_type = genomic DNA

SEQ ID NO: 112 - DNA, 83 bp, Arabidopsis thaliana
  source 1..83: mol_type = genomic DNA
taaatttgattcgttgatagggctaaagagatgtgggaaaagttgtagactgatggatgaattatttgagccctaatgtgaac

SEQ ID NO: 113 - DNA, 83 bp, Arabidopsis thaliana
  source 1..83: mol_type = genomic DNA
taaatttgattcgttgatagggctaaagagatgtgggaaaagttgtagacagatggatgaattatttgagccctaatgtgaac

SEQ ID NO: 114 - DNA, 86 bp, Arabidopsis thaliana; identical to SEQ ID NO: 105
  source 1..86: mol_type = genomic DNA

SEQ ID NO: 115 - DNA, 86 bp, Arabidopsis thaliana; identical to SEQ ID NO: 107
  source 1..86: mol_type = genomic DNA

SEQ ID NO: 116 - DNA, 86 bp, Arabidopsis thaliana; identical to SEQ ID NO: 105
  source 1..86: mol_type = genomic DNA

SEQ ID NO: 117 - DNA, 78 bp, Arabidopsis thaliana
  source 1..78: mol_type = genomic DNA
taaatttgattcgttgatagggctaaagagatgtgggctaaacatagatggatgaattatttgagccctaatgtgaac